(19) United States
(12) Patent Application Publication
(10) Pub. No.: US 2005/0049245 A1
(43) Pub. Date: Mar. 3, 2005

(54) Heteroaryl-ethanolamine derivatives as antiviral agents

(76) Inventors: Mark E. Schnute, Kalamazoo, MI (US); Michele M. Cudahy, Plainwell, MI (US); MariJean Eggen, Portage, MI (US); David John Anderson, Kalamazoo, MI (US); Thomas M. Judge, Otsego, MI (US); Euibong J. Kim, Kalamazoo, MI (US); Sarah A. Collier, Kalamazoo, MI (US)

Correspondence Address: Pfizer Inc., 301 Henrietta, Kalamazoo, MI, 49001, US

(21) Appl. No.: 10/649,208

(22) Filed: Aug. 27, 2003

Related U.S. Application Data
(60) Provisional application No. 60/408,206, filed on Sep. 4, 2002.

(51) Int. Cl. C07D 498/02; A61K 31/541; A61K 31/5377; A61K 31/496; A61K 31/4743

(52) U.S. Cl. 514/227.8; 514/234.2; 514/253.04; 514/301; 544/60; 544/126; 544/362; 546/114

(57) ABSTRACT

The present invention provides a compound of formula as described herein, which are useful as antiviral agents, in particular, as agents against viruses of the herpes family.

### CROSS REFERENCE

This application claims the benefit of the following provisional application: U.S. Ser. No. 60/408,206, filed Sep. 4, 2002 under 35 USC 119(e)(i), which is incorporated herein by reference in its entirety

### FIELD OF THE INVENTION

The present invention discloses five-(5) membered heteroaryl-ethanolamine derivatives, and more specifically, provides compounds of formula (I) described herein below. These compounds are useful as antiviral agents, in particular, as agents against viruses of the herpes family.

### BACKGROUND OF THE INVENTION

The herpesviruses comprise a large family of double stranded DNA viruses. They are also a source of the most common viral illnesses in man. Eight of the herpes viruses, herpes simplex virus types 1 and 2 (HSV-1 and HSV-2), varicella zoster virus (VZV), human cytomegalovirus (HCMV), Epstein-Barr virus (EBV), and human herpes viruses 6, 7, and 8 (HHV-6, HHV-7, and HHV-8), have been shown to infect humans.

HSV-1 and HSV-2 cause herpetic lesions on the lips and genitals, respectively. They also occasionally cause infections of the eye and encephalitis. HCMV causes birth defects in infants and a variety of diseases in immunocompromised patients such as retinitis, pneumonia, and gastrointestinal disease. VZV is the causitive agent of chicken pox and shingles. EBV causes infectious mononucleosis. It can also cause lymphomas in immunocompromised patients and has been associated with Burkitt's lymphoma, nasopharyngeal carcinoma, and Hodgkins disease. HHV-6 is the causative agent of roseola and may be associated with multiple sclerosis and chronic fatigue syndrome. HHV-7 disease association is unclear, but it may be involved in some cases of roseola. HHV-8 has been associated with Karposi's sarcoma, body cavity based lymphomas, and multiple myeloma.

Infection by or reactivation of herpesviruses is associated with several cardiovascular diseases or conditions in the host such as atherosclerosis and restenosis resulting in inflammation of coronary vessel walls. It is thought that in many patients suffering from restenosis following coronary atherectomy viral infection particularly by CMV plays an important role in the proliferation of the disease. Atherosclerosis is believed to be be associated with the overall infectious disease burden in the host and particularly by the herpesviruses such as HSV, CMV, and EBV.

Infection in the animal population (livestock and companion) by strains of herpesviruses is endemic including cattle (Bovine herspesvirus 1-5, BHV), sheep (Ovine herpesvirus 1 and 2), dog (Canine herpesvirus 1), horse (Equine herpesvirus 1-8, EHV), cat (Feline herpesvirus 1, FHV), swine (pseudorabies virus, PRV), and many species of fowl. In the case of bovine herpesvirus infection, animals may suffer from ocular, respiratory, or digestive disorders. Pseudorabies is an extremely contagious viral pathogen infecting several species such as cattle, horses, dogs, cats, sheep, and goats leading to rapid death. The virus is benign in adult swine, however, it remains contagious and leads to high mortality in pigs under three weeks. Infection of horses by equine herpesvirus may lead to neurological syndromes, respiratory disease, and neonatal disease. Herpesvirus infection in cats leads to the disease known as feline viral rhinotracheitis (FVR) which is characterized by rhinitis, tracheitis, laryngitis, and conjunctivitis.

Due to the unique position of the five-(5) membered heteroaryl substitutent on the formula I described herein below, compounds of the present invention demonstrate unexpected activity against the above reference herpesviral infections, particularly, human cytomegaloviral infection.

### Information Disclosure

U.S. Pat. No. 6,239,142 disclosed compounds and their use to treat herpesvirus infections.

WO02/06513 disclosed method of screening 4-hydroxyquinline, 4-oxo-dihydroquinoline, and 4-oxo-dihydrothienopyridine derivatives as non-nucleoside herpesvirus DNA polymerase inhibitors.

Tetrahedron Lett. 1983, 24, 3233-3236 describes conditions to transform tertiary N-benzylamines into benzylchlorides.

WO95/28405 disclosed bicyclic thiophene derivatives and use as gonadotropin releasing hormone Antagonists).

EP 443568 disclosed fused thiophene derivatives, their production and use.

WO02/04445 disclosed a variety of tricyclic core structures which have antiviral activity against herpesviruses.

WO02/04444, WO02/04443, and WO02/04422 disclosed a variety of bicyclic core structures which have antiviral activity against herpesviruses.

U.S. Pat. No. 6,248,739 disclosed compounds in which the core structure is a quinoline and useful as antivirals against herpesviruses.

WO00/53178, WO00/53179, WO00/53180, WO00/53181, WO00/53185, and WO00/53602 disclosed 6-azaindole compounds as antagonists of gonadotropin releasing hormone.

U.S. Pat. No. 6,346,534 and WO00/69859 disclosed imidazo- and pyrrolo[1,2-a]pyrimid-4-ones as gonadotropin-releasing hormone receptor antagonists.

WO 94/12461 disclosed a variety of bicyclic core structures useful as potential treatments of AIDS, asthma, arthritis, and other inflammatory diseases.

### SUMMARY OF THE INVENTION

The present invention provides a compound of formula I 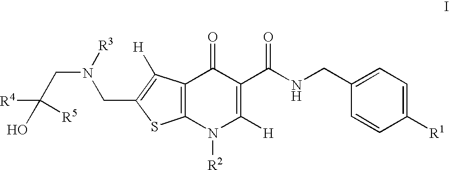
its enantiomeric, diasteromeric or tautomeric isomer, or a pharmaceutically acceptable salt thereof wherein,
R¹ is
(a) Cl,
(b) Br,
(c) F, or
(d) CN;
   R² is
(a) C₁₋₄alkyl optionally substituted by one or more OH or C₁₋₄alkoxy, or
(b) (CH₂)ₘOCH₂CH₂OH;
   R³ is C₁₋₂alkyl;
   R⁴ is a five-(5) membered heteroaryl bonded via a carbon atom having 1, 2, or 3 heteroatoms selected from the group consisting of O, S(O)ₘ, and N—W, wherein R⁴ is optionally fused to a benzene or pyridine ring, and optionally substituted with one or more R⁶;
   wherein W is absence, H, or C₁₋₄alkyl;
   R⁵ is
(a) H, or
(b) C₁₋₂alkyl optionally substituted by OH;
   R⁶ is
(a) halo,
(b) OCF₃,
(c) cyano,
(d) nitro,
(e) CONR⁷R⁸,
(t) NR⁷R⁸,
(g) C₁₋₇alkyl, which is optionally partially unsaturated and is optionally substituted by one or more R⁹,
(h) O(CH₂CH₂O)ₙR¹⁰,
(i) OR¹⁰,
(j) CO₂R¹⁰, or
(k) phenyl optionally substituted by halo, C₁₋₇alkyl, or C₁₋₇alkoxy;
   R⁷ and R⁸ are independently
(a) H,
(b) phenyl optionally substituted by halo, C₁₋₇alkyl, or C₁₋₇alkoxy,
(c) C₁₋₇alkyl which is optionally substituted by one or more OR¹⁰, phenyl, or halo substituents,
(d) C₃₋₈cycloalkyl,
(e) (C═O)R¹¹, or
(f) R⁷ and R⁸ together with the nitrogen to which they are attached form a het, wherein het is a five-(5), or six-(6) membered heterocyclic ring having one (1), two (2), or three (3) heteroatoms selected from the group consisting of oxygen, sulfur, or nitrogen, wherein het is optionally substituted with C₁₋₄ alkyl;
   R⁹ is
(a) oxo,
(b) phenyl optionally substituted by halo, C₁₋₇alkyl, or C₁₋₇alkoxy,
(c) OR¹⁰,
(d) O(CH₂CH₂)OR¹⁰,
(e) SR¹⁰,
(f) NR⁷R⁸,
(g) halo,
(h) CO₂R¹⁰,
(i) CONR¹⁰OR¹⁰, or
(j) C₃₋₈cycloalkyl optionally substituted by OR¹⁰;
   R¹⁰ is
(a) H,
(b) C₁₋₇alkyl,
(c) C₃₋₈cycloalkyl, or
(d) phenyl optionally substituted by halo, C₁₋₇alkyl, or C₁₋₇alkoxy,
   R¹¹ is
(a) C₁₋₇alkyl,
(b) C₃₋₈cycloalkyl, or
(c) phenyl optionally substituted by halo, C₁₋₇alkyl, or C₁₋₇alkoxy,
   n is 1, 2, 3, 4 or 5; and
   each m is independently 1 or 2.

In another aspect, the present invention also provides:

A pharmaceutical composition which comprises a pharmaceutically acceptable carrier and an effective amount of a compound of formula I,
a method of treating and preventing herpesviral infections in a mammal comprising administering to a mammal in need thereof a compound of formula I, or a pharmaceutically acceptable salt thereof,
a method for inhibiting a viral DNA polymerase comprising contacting, in vivo or in vitro, the polymerase with an effective inhibitory amount of a compound of formula I, or a pharmaceutically acceptable salt thereof,
a compound of formula I or a pharmaceutically acceptable salt thereof for use in medical treatment or prevention of a herpesviral infection in a mammal.

The invention also provides novel intermediates and processes disclosed herein that are useful for preparing compounds of formula I.

### DETAILED DESCRIPTION OF THE INVENTION

For the purpose of the present invention, the carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, (C₁₋₇)alkyl refers to alkyl of one to seven carbon atoms, inclusive, or methyl, ethyl, propyl, butyl, pentyl, hexyl, and heptyl, straight and branched forms thereof.

The term “halo” or “halogen” refers to the elements fluorine (F), chlorine (Cl), bromine (Br) and iodine (I).

The term “C₃₋₈cycloalkyl” refers to a non-aromatic carbocyclic ring having from 3 to 8 carbon atoms.

The term “alkoxy” refers to the group RO—, wherein R is alkyl or cycloalkyl as defined above.

The term “heteroaryl” refers to aromatic heterocyclic groups.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, tautomeric, or stereoisomeric form, or mixture thereof, of a compound of the invention, which possesses the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine antiviral activity using the standard tests described herein, or using other similar tests which are well known in the art.

The compounds of the present invention are generally named according to the IUPAC or CAS nomenclature system.

“Pharmaceutically acceptable salts” refers to those salts which possess the biological effectiveness and properties of the parent compound and which are not biologically or otherwise undesirable.

“Mammal” refers to human and animals. Animals specifically refer to, for example, food animals or companion animals.

“Optionally” or “may be” means that the subsequently described event or circumstance may, but need not, occur, and that the description includes instances where the event or circumstance occurs and instances in which it does not.

A “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and claims includes both one and more than one such carrier.

Specifically, formula I of the present invention has a stereogenic center as shown in formula IA:

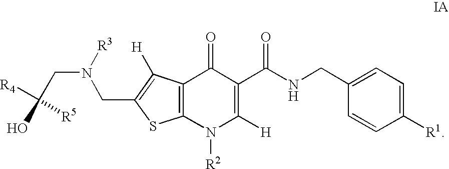

Specifically, a composition comprising over 51% of a compound of formula IA.

Specifically, a composition comprising over 75% of a compound of formula IA.

Specifically, a composition comprising over 90% of a compound of formula IA.

Specifically, a composition comprising over 98% of a compound of formula IA.

Specifically, R¹ is chloro.

Specifically, R² is C₁₋₃alkyl.

Specifically, R² is methyl, ethyl, or n-propyl.

Specifically, R² is methyl.

Specifically, R² is C₁₋₃alkyl substituted with one or two hydroxy.

Specifically, R² is 2-hydroxyethyl, 3-hydroxypropyl, or 2,3-dihydroxypropyl.

Specifically, R² is C₁₋₄alkyl substituted by C₁₋₄alkoxy.

Specifically, R² is C₁₋₄alkyl substituted by methoxy.

Specifically, R² is 2-methoxyethyl.

Specifically, R² is CH₂CH₂OCH₂CH₂OH.

Specifically, R³ is methyl.

Specifically, R³ is ethyl.

Specifically, R⁴ is a five-(5) membered heteroaryl bonded via a carbon atom having one (1) or two (2) heteroatoms selected from the group consisting of O, S, and N—W.

Specifically W is absence.

Specifically W is H.

Specifically W is methyl, ethyl, propyl, butyl, 2-methylpropyl.

Specifically, R⁴ is 2-furyl, 3-furyl, thien-2-yl, thien-3-yl, 1H-pyrrol-2-yl, 1H-pyrrol-3-yl, 1H-imidazol-4-yl, 1H-imidazol-2-yl, 1,3-thiazol-2-yl, 1H-pyrazol-5-yl, 1-methyl-1H-pyrrol-2-yl, 1-ethyl-1H-pyrrol-2-yl, 1-propyl-1H-pyrrol-2-yl, 1-methyl-1H-imidazol-4-yl, 1-methyl-1H-imidazol-2-yl, 1-ethyl-1H-imidazol-4-yl, or 1-ethyl-1H-imidazol-2-yl.

Specifically, R⁴ is a five-(5) membered heteroaryl bonded via a carbon atom having one (1) or two (2) heteroatoms selected from the group consisting of O, S, and N—W, wherein R⁴ is substituted by R⁶.

Specifically, R⁴ is 5-methyl-2-furyl, 2,5-dimethyl-3-furyl, 4,5-dimethyl-2-furyl, 4-methyl-2-furyl, 5-hydroxymethyl-2-furyl, 5-((dimethylamino)methyl)-2-furyl, 5-ethyl-2-furyl, 5-bromo-2-furyl, 4,5-dibromo-2-furyl, 5-chloro-2-furyl, 5-trifluoromethyl-2-furyl, 5-phenyl-2-furyl, 4-phenyl-2-furyl, 5-(2-chlorophenyl)-2-furyl, 5-(3-chlorophenyl)-2-furyl, 5-(4-chlorophenyl)-2-furyl, 5-(2,4-dichlorophenyl)-2-furyl, 5-(2,5-dichlorophenyl)-2-furyl, 5-(2,4,6-trichlorophenyl)-2-furyl, 5-cyanothien-2-yl, 4-bromothien-2-yl, or 5-chlorothien-2-yl.

Specifically, R⁴ is a five-(5) membered heteroaryl bonded via a carbon atom having one (1) or two (2) heteroatoms selected from the group consisting of O, S, and N—W, wherein R⁴ is fused to a benzene or pyridine ring.

Specifically, R⁴ is benzofuran-2-yl, benzofuran-3-yl, benzothien-2-yl, benzothien-3-yl, 1H-indol-3-yl, 1H-indol-2-yl, 1,3-benzothiazol-2-yl, furo[2,3-b]-pyridin-2-yl, furo[2,3-c]pyridin-2-yl, furo[3,2-c]pyridin-2-yl, furo[3,2-b]pyridin-2-yl, furo[2,3-b]pyridin-3-yl, furo[2,3-c]pyridin-3-yl, furo[3,2-c]pyridin-3-yl, furo[3,2-b]pyridin-3-yl, 1-methyl-1H-indol-2-yl, 1-ethyl-1H-indol-2-yl Specifically, R⁴ is a five-(5) membered heteroaryl bonded via a carbon atom having one (1) or two (2) heteroatoms selected from the group consisting of O, S, and N—W, wherein R⁴ is fused to a benzene or pyridine ring, and is substituted with one or more W;
Specifically, R⁴ is 3-chloro-1-benzofuran-2-yl, 2-phenyl-1H-indol-3-yl, 2-(4-fluorophenyl)-1H-indol-3-yl, 5-fluoro-1H-indol-3-yl, 2-methyl-1H-indol-3-yl, 5-methyl-1H-indol-3-yl, 6-methyl-1H-indol-3-yl, 7-methyl-1H-indol-3-yl, 3-methyl-1-benzothien-2-yl, 3-phenyl-1H-pyrazol-4-yl, or 1,3-dimethyl-1H-pyrazol-4-yl.

Specifically, R⁴ is 1-methyl-1H-1,2,4-triazol-5-yl.

Specifically, R⁵ is hydrogen.

Specifically, R⁵ is methyl or ethyl.

Specifically, R⁵ is hydroxymethyl, 1-hydroxyethyl, or 2-hydroxyethyl.

Specifically, R⁶ is OH, halo, C₁₋₄alkyl, C₁₋₄alkoxy, cyano, nitro, OCF₃, NR⁷R⁸, phenyl, or CONR⁷R⁸.

Specifically, R⁶ is C₁₋₇alkyl which is optionally substituted by one or more R⁹.

Specifically, R⁶ is methyl, ethyl, hydroxymethyl, dimethylaminomethyl, trifluoromethyl, or benzyl.

Specifically, R⁶ is phenyl optionally substituted by halo, C₁₋₇alkyl, or C₁₋₇alkoxy;
Specifically, R⁶ is morpholine, piperidine, piperazine, or pyrrolidine.

Examples of the present invention include, but are not limited to the following:
(1) rac-N-(4-chlorobenzyl)-2-(((2-(2-furyl)-2-hydroxyethyl)(methyl)amino)-methyl)-7-methyl-4-oxo-4,7-dihydrothieno[2,3-b]pyridine-5-carboxamide,
(2) (+)—N-(4-chlorobenzyl)-2-((((R)-2-(2-furyl)-2-hydroxyethyl)(methyl)amino)-methyl)-7-methyl-4-oxo-4,7-dihydrothieno[2,3-b]pyridine-5-carboxamide,
(3) rac-N-(4-chlorobenzyl)-2-(((2-hydroxy-2-(5-methyl-2-furyl)ethyl)(methyl)-amino)methyl)-7-methyl-4-oxo-4,7-dihydrothieno[2,3-b]pyridine-5-carboxamide,
(4) rac-N-(4-chlorobenzyl)-2-(((2-(3-furyl)-2-hydroxyethyl)(methyl)amino)-methyl)-7-methyl-4-oxo-4,7-dihydrothieno[2,3-b]pyridine-5-carboxamide,
(5) rac-2-(((2-(1-benzofuran-2-yl)-2-hydroxyethyl)(methyl)amino)methyl)-N-(4-chlorobenzyl)-7-methyl-4-oxo-4,7-dihydrothieno[2,3-b]pyridine-5-carboxamide,
(6) rac-N-(4-chlorobenzyl)-2-(((2-hydroxy-2-thien-2-ylethyl)(methyl)amino)-methyl)-7-methyl-4-oxo-4,7-dihydrothieno[2,3-b]pyridine-5-carboxamide,
(7) rac-N-(4-chlorobenzyl)-2-(((2-hydroxy-2-(1H-pyrrol-2-yl)ethyl)(methyl)-amino)methyl)-7-methyl-4-oxo-4,7-dihydrothieno[2,3-b]pyridine-5-carboxamide,
(8) rac-N-(4-Chlorobenzyl)-2-(((2-hydroxy-2-(1-methyl-1H-pyrrol-2-yl)ethyl)-(methyl)amino)methyl)-7-methyl-4-oxo-4,7-dihydrothieno[2,3-b]pyridine-5-carboxamide,
(9) rac-N-(4-chlorobenzyl)-2-(((2-hydroxy-2-(1-methyl-1H-imidazol-4-yl)ethyl)(methyl)amino)methyl)-7-methyl-4-oxo-4,7-dihydrothieno[2,3-b]pyridine-5-carboxamide,
(10) rac-N-(4-chlorobenzyl)-2-(((2-hydroxy-2-(1H-imidazol-4-yl)ethyl) (methyl) amino)methyl)-7-methyl-4-oxo-4,7-dihydrothieno[2,3-b]pyridine-5-carboxamide,
(11) rac-N-(4-chlorobenzyl)-2-(((2-hydroxy-2-(1H-indol-3-yl)ethyl)(methyl)amino)-methyl)-7-methyl-4-oxo-4,7-dihydrothieno[2,3-b]pyridine-5-carboxamide,
(12) rac-N-(4-chlorobenzyl)-2-(((2-(2,5-dimethyl-3-furyl)-2-hydroxyethyl)(methyl)-amino)methyl)-7-methyl-4-oxo-4,7-dihydrothieno[2,3-b]pyridine-5-carboxamide,
(13) rac-2-(((2-(1-benzothien-3-yl)-2-hydroxyethyl)(methyl)amino)methyl)-N-(4-chlorobenzyl)-7-methyl-4-oxo-4,7-dihydrothieno[2,3-b]pyridine-5-carboxamide,
(14) rac-N-(4-chlorobenzyl)-2-(((2-hydroxy-2-(1-methyl-1H-indol-2-yl)ethyl)(methyl)amino)methyl)-7-methyl-4-oxo-4,7-dihydrothieno[2,3-b]pyridine-5-carboxamide,
(15) rac-N-(4-chlorobenzyl)-2-(((2-(5-cyanothien-2-yl)-2-hydroxyethyl)(methyl)-amino)methyl)-7-methyl-4-oxo-4,7-dihydrothieno[2,3-b]pyridine-5-carboxamide,
(16) rac-N-(4-chlorobenzyl)-2-(((2-hydroxy-2-(1,3-thiazol-2-yl)ethyl)(methyl)-amino)methyl)-7-methyl-4-oxo-4,7-dihydrothieno[2,3-b]pyridine-5-carboxamide,
(17) rac-2-(((2-(1,3-benzothiazol-2-yl)-2-hydroxyethyl)(methyl)amino)methyl)-N-(4-chlorobenzyl)-7-methyl-4-oxo-4,7-dihydrothieno[2,3-b]pyridine-5-carboxamide,
(18) rac-N-(4-chlorobenzyl)-2-(((2-hydroxy-2-(1H-pyrazol-5-yl)ethyl)(methyl)-amino)methyl)-7-methyl-4-oxo-4,7-dihydrothieno[2,3-b]pyridine-5-carboxamide,
(19) N-(4-chlorobenzyl)-2-((((2R)-2-hydroxy-2-(1H-pyrazol-5-yl)ethyl)(methyl)-amino)methyl)-7-methyl-4-oxo-4,7-dihydrothieno[2,3-b]pyridine-5-carboxamide,
(20) N-(4-chlorobenzyl)-7-ethyl-2-((((2R)-2-(2-furyl)-2-hydroxyethyl)(methyl)-amino)methyl)-4-oxo-4,7-dihydrothieno[2,3-b]pyridine-5-carboxamide,
(21) N-(4-chlorobenzyl)-2-((((2R)-2-(2-furyl)-2-hydroxyethyl)(methyl)amino)-methyl)-7-(2-methoxyethyl)-4-oxo-4,7-dihydrothieno[2,3-b]pyridine-5-carboxamide,
(22) rac-2-(((2-(1-benzofuran-2-yl)-2-hydroxyethyl)(methyl)amino)methyl)-N-(4-chlorobenzyl)-7-ethyl-4-oxo-4,7-dihydrothieno[2,3-b]pyridine-5-carboxamide,
(23) rac-2-(((2-(1-benzofuran-2-yl)-2-hydroxyethyl)(methyl)amino)methyl)-N-(4-chlorobenzyl)-7-propyl-4-oxo-4,7-dihydrothieno[2,3-b]pyridine-5-carboxamide,
(24) rac-2-(((2-(1-benzofuran-2-yl)-2-hydroxyethyl)(methyl)amino)methyl)-N-(4-chlorobenzyl)-7-(2-methoxyethyl)-4-oxo-4,7-dihydrothieno[2,3-b]pyridine-5-carboxamide,
(25) rac-2-(((2-(1-benzofuran-2-yl)-2-hydroxyethyl)(methyl)amino)methyl)-N-(4-chlorobenzyl)-7-(2,3-dihydroxypropyl)-4-oxo-4,7-dihydrothieno[2,3-b]pyridine-5-carboxamide,
(26) N-(4-chlorobenzyl)-7-(2,3-dihydroxypropyl)-2-((((2R)-2-(2-furyl)-2-hydroxyethyl)(methyl)amino)methyl)-4-oxo-4,7-dihydrothieno[2,3-b]pyridine-5-carboxamide,
(27) N-(4-chlorobenzyl)-2-((((2R)-2-(2-furyl)-2-hydroxyethyl)(methyl)amino)-methyl)-7-(3-hydroxypropyl)-4-oxo-4,7-dihydrothieno[2,3-b]pyridine-5-carboxamide,
(28) rac-2-(((2-(1-benzofuran-2-yl)-2-hydroxyethyl)(methyl)amino)methyl)-N-(4-chlorobenzyl)-7-(3-hydroxypropyl)-4-oxo-4,7-dihydrothieno[2,3-b]pyridine-5-carboxamide,
(29) rac-2-(((2-(1-benzofuran-2-yl)-2-hydroxyethyl)(methyl)amino)methyl)-N-(4-chlorobenzyl)-7-(2-hydroxyethyl)-4-oxo-4,7-dihydrothieno[2,3-b]pyridine-5-carboxamide,
(30) rac-N-(4-chlorobenzyl)-2-(((2-(4,5-dimethyl-2-furyl)-2-hydroxyethyl)(methyl)-amino)methyl)-7-methyl-4-oxo-4,7-dihydrothieno[2,3-b]pyridine-5-carboxamide,
(31) rac-N-(4-chlorobenzyl)-2-(((2-(5-phenyl-2-furyl)-2-hydroxyethyl)(methyl)-amino)methyl)-7-methyl-4-oxo-4,7-dihydrothieno[2,3-b]pyridine-5-carboxamide,
(32) N-(4-chlorobenzyl)-2-((((2R)-2-(2-furyl)-2-hydroxyethyl)(methyl)amino)-methyl)-4-oxo-7-propyl-4,7-dihydrothieno[2,3-b]pyridine-5-carboxamide,
(33) N-(4-chlorobenzyl)-2-(((2-(5-chloro-2-furyl)-2-hydroxyethyl)(methyl)amino)-methyl)-7-methyl-4-oxo-4,7-dihydrothieno[2,3-b]pyridine-5-carboxamide,
(34) N-(4-chlorobenzyl)-2-((((2R)-2-(2-furyl)-2-hydroxyethyl)(methyl)amino)-methyl)-7-(2-hydroxyethyl)-4-oxo-4,7-dihydrothieno[2,3-b]pyridine-5-carboxamide,
(35) N-(4-chlorobenzyl)-2-((((2R)-2-(2-furyl)-2-hydroxyethyl)(methyl)amino)-methyl)-7-(2-(2-hydroxyethoxy)ethyl)-4-oxo-4,7-dihydrothieno[2,3-b]pyridine-5-carboxamide,
(36) rac-N-(4-chlorobenzyl)-2-(((2-hydroxy-2-(1H-imidazol-2-yl)ethyl)(methyl)-amino)methyl)-7-methyl-4-oxo-4,7-dihydrothieno[2,3-b]pyridine-5-carboxamide, and pharmaceutically acceptable salts thereof.

Charts A-O describe the preparation of the compounds of Formula (I) of the present invention. All of the starting materials are prepared by procedures described in these charts, by procedures well known to one of ordinary skill in organic chemistry or can be obtained commercially. All of the final compounds of the present invention are prepared by procedures described in these charts or by procedures analogous thereto, which would be well known to one of ordinary skill in organic chemistry.

Compounds of Formula (I) are prepared as described in Chart A. Compounds of the formula A.1 in which X is a leaving group (e.g. mesylate, chloride, or bromide) are treated with a secondary amine of the formula R⁴R⁵C(OH)CH₂NH(R³) in the presence of a non-nucleophilic base (e.g. diisopropylethylamine) in a polar solvent (e.g. DMF) to afford products of the formula A.2. It would be understood by those skilled in the art that in some cases transient protection of hydroxyl and other Lewis basic or acidic functionality present in R⁴R⁵C(OH)CH₂NH(R³) may be required to facilitate the coupling described in Chart A for which procedures are well established (Greene, T. W.; Wuts, P. G. M. Protective Groups in Organic Synthesis, 1999).

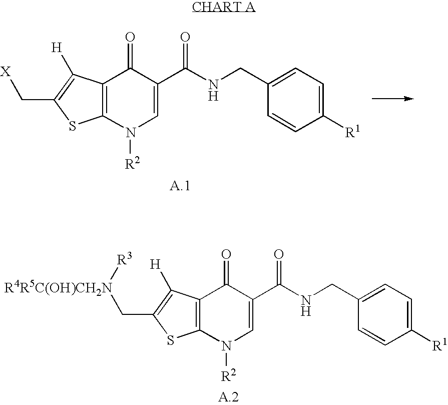

Alternatively, compounds of Formula (I) are prepared as described in Chart B. Compounds of the formula A.1 in which X is a leaving group (e.g. mesylate, chloride, or bromide) are treated with a primary amine of the formula R⁴R⁵C(OH)CH₂NH₂ in the presence of a non-nucleophilic base (e.g. diisopropylethylamine) in a polar solvent (e.g. DMF) to afford products of the formula B. 1. The resulting secondary amine is then alkylated by reactions generally known by those skilled in the art such as (1) the reaction of B. 1 with a corresponding alkylhalide, dialkylsulfonate, or alkylarylsulfonate or (2) the reaction of B.1 with an aldehyde (e.g. formaldehyde or acetaldehyde) in the presence of a reducing agent (e.g. sodium cyanoborohydride or sodium triacetoxyborohydride) to afford compounds of the general formula A.2.

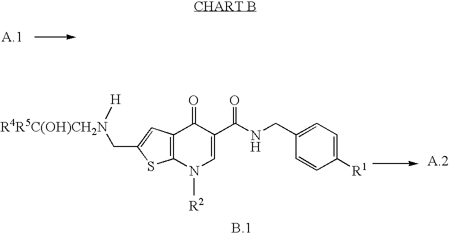

Alternatively, compounds of Formula (I) are prepared as described in Chart C. Compounds of the formula A.1 in which X is a leaving group (e.g. mesylate, chloride, or bromide) are treated with an alkyl primary amine (e.g. methylamine or ethylamine) in the presence of a non-nucleophilic base (e.g. diisopropylethylamine) in a polar solvent (e.g. DMF) to afford products of the formula C.1. The resulting secondary amine is then treated with an electrophile either of the formula R⁴R⁵C(OH)CH₂X (where X is Cl, Br) in the presence of a non-nucleophilic base (e.g. diisopropylethyl-amine) in a polar solvent (e.g. DMF) or with an epoxide to afford products of the formula A.2. Alternatively, compounds of the formula C.1 are alkylated with 2-haloketones of the formula R⁴C(O)CH₂X (where X is Cl, Br) according to Chart D to afford products of the formula D. 1. The resulting amino ketones are then reduced with an appropriate achiral or chirally-modified reducing agent (e.g. NaBH₄ or diisopinocamphenylchloroborane) to provide compounds of the formula A.2.

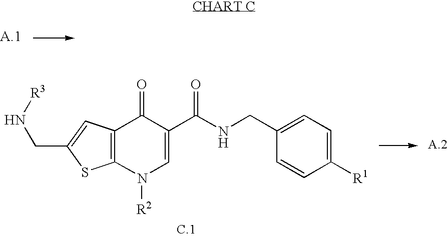

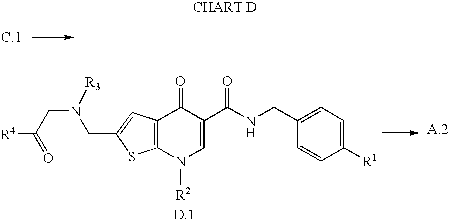

The precursors A.1 are available from the corresponding alcohols (Y═OH) by treatment with methanesulfonyl chloride in the presence of an organic base (e.g. pyridine or 2,4,6-collidine) and if needed an activating agent (e.g. DMAP), Chart E. Alternatively, compounds of the formula A.1 are available by treatment of a tertiary amino derivative (e.g. Y═N(CH₃)₂ or 4-morpholinyl) with ethyl chloroformate in an appropriate solvent (e.g. chloroform, dichloromethane, 1,2-dichloroethane, or benzene). 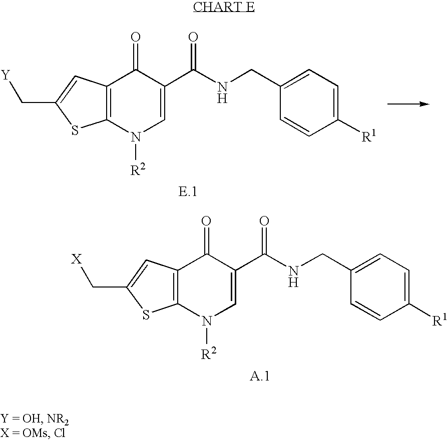
Subsequently, compounds of the general formula E.1 are prepared according to procedures described in U.S. Pat. No. 6,239,142 or exemplified in Charts F, G, and H below.

As described in Chart F, 3-bromo-2-chlorothiophene (F.1) is metalated with lithium diisopropyl amide in tetrahydrofuran at low temperature followed by addition to paraformaldehyde to provide alcohol F.2. The free hydroxyl is protected employing common methodology (Greene, T. W.; Wuts, P. G. M. Protective Groups in Organic Synthesis, 1999) such as the tert-butyldimethylsilyl ether (TBS) by treatment with the corresponding silyl chloride and a weak base (e.g. imidazole) in a polar solvent (e.g. DMF). Metalation of F.3 with n-butyl lithium followed by addition to N-methoxy-N-methylacetamide provides the methyl ketone F.4. Condensation of F.4 with diethyl carbonate in the presence of a strong base (e.g. sodium hydride) affords ketoester F.5. Compound F.5 is then refluxed in a mixture of acetic anhydride and triethylorthoformate to afford an intermediate enol ether which is then condensed with a primary amine or aniline (e.g. R²NH₂) to provide a compound of the formula F.6. The resulting enamines are cyclized by heating in the presence of a base (e.g. sodium hydride, potassium carbonate, or potassium tert-butoxide) in an appropriate solvent (e.g. THF, DMF, or tert-butanol) to provide F.7. Esters of the formula F.7 are converted to amides of the general formula F.8 by either (a) treatment with a substituted benzylamine (e.g. 4-chlorobenzylamine, 4-fluorobenzylamine, or 4-bromobenzylamine) at high temperature or (b) saponification by treatment with an inorganic base such as sodium hydroxide to afford the corresponding carboxylic acid which is then coupled with a substituted benzylamine mediated by 1,1′-carbonyl-diimidazole (or other suitable carboxylic acid activating agent). Subsequent deprotection of the hydroxyl protecting group to afford E.1 is accomplished through common procedures such as treatment with tetrabutylammonium fluoride in the case of silyl ether protection.

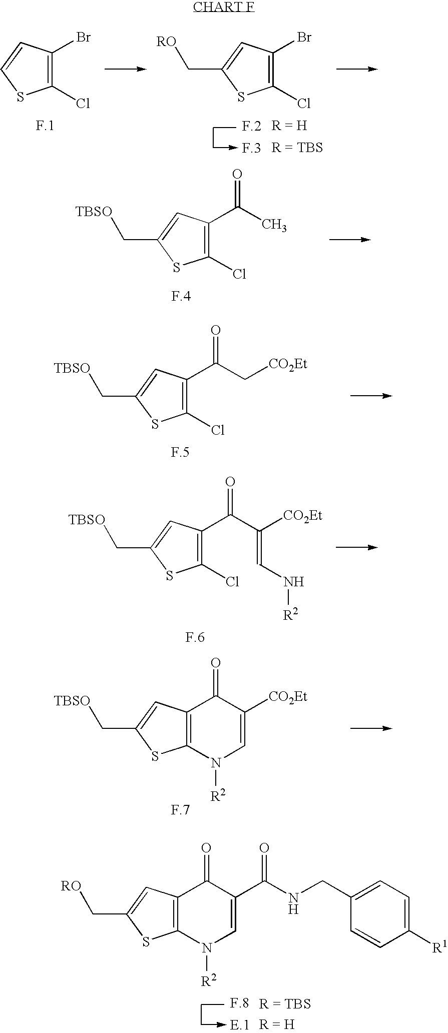

Compounds of formula E.1 (Y═NR²) may be prepared as described in Chart G. 3-Bromo-2-chlorothiophene (F.1) is metalated with lithium diisopropyl amide in tetrahydrofuran at low temperature and condensed with N,N-dimethylformamide to afford the carboxaldehyde G.1. Reductive amination of G.1 by treating with an amine (e.g. morpholine), acetic acid, and an appropriate reducing agent (e.g. sodium triacetoxyborohydride) affords thiophenes of the formula G.2. Metalation of G.2 with n-butyl lithium followed by addition to N-methoxy-N-methylacetamide provides the methyl ketone G.3. Condensation of G.3 with diethyl carbonate in the presence of a strong base (e.g. sodium hydride) affords ketoester G.4. The resulting ketoester is then treated with a benzylamine (e.g. 4-chlorobenzylamine, 4-fluorobenzylamine, or 4-bromobenzylamine) in refluxing xylene to provide ketoamides of the formula G.5. Compound G.5 is then refluxed in a mixture of acetic anhydride and triethylorthoformate to afford an intermediate enol ether which is then condensed with a primary amine or aniline (e.g. R²NH₂) to provide a compound of the formula G.6. The resulting enamines are cyclized by heating in the presence of a base (e.g. sodium hydride, potassium carbonate, or potassium tert-butoxide) in an appropriate solvent (e.g. THF, DMF, or tert-butanol).

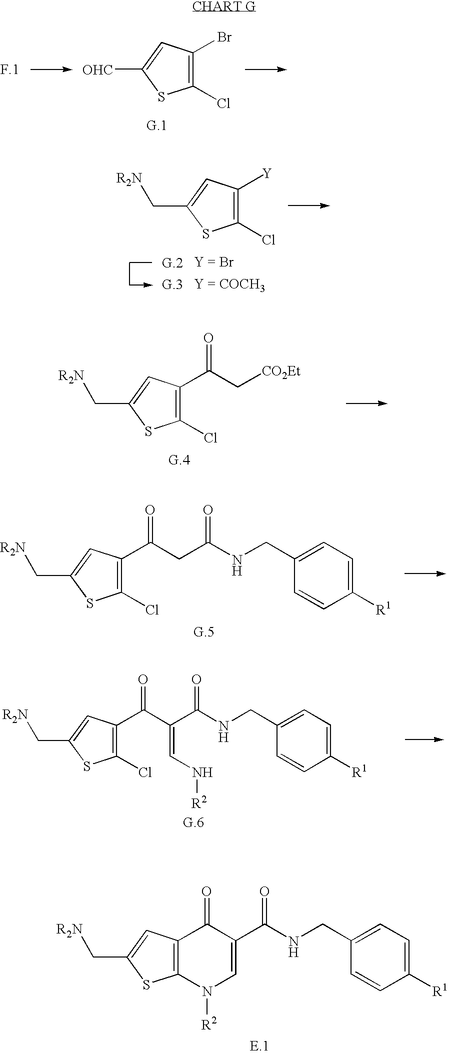

Alternatively, compounds of formula E.1 (Y═OH) may be prepared as described in Chart H. Ethyl 4-hydroxythieno[2,3-b]pyridine-5-carboxylate (J. Heterocyclic Chem. 1977, 14, 807) is metallated with from two to six equivalents of lithium diisopropylamide at low temperature and is then reacted with dimethylformamide to provide compound H.2. Treatment of H.2 with an appropriate reducing agent (e.g. NaBH₄) in a polar solvent (e.g. ethanol) affords the alcohol H.3. The resulting ester is then reacted with a substituted benzylamine (e.g. 4-chlorobenzylamine, 4-fluorobenzylamine, or 4-bromobenzylamine) at high temperature or under other common amide forming conditions well known to those skilled in the art to provide compounds of the formula H.4. Compound H.4 is alkylated at the ring nitrogen by treatment with an optionally substituted alkyl halide or alkyl sulfonate ester in the presence of a base (e.g. potassium carbonate) or by reaction with an optionally substituted alkanol under Mitsunobu conditions to afford compounds of the general formula E.1. Specific examples of such alkyl halides used in this reaction include but are not limited to iodomethane, iodoethane, 1-iodopropane, 1-iodobutane, and 1-bromo-2-methoxyethane. It would be understood by those skilled in the art that in some cases transient protection of hydroxyl functionality present in the R²X (X=halo or sulfonate) or R²OH reagent used in the above step may be required to facilitate the coupling described in Chart H or subsequent chemistry described in Charts A-E. Specific examples of such protected-hydroxyalkyl halides used in this reaction include but are not limited to 2-(2-bromoethoxy)tetrahydro-2H-pyran, 2-(3-bromopropoxy)tetrahydro-2H-pyran, 4-(bromomethyl)-2,2-dimethyl-1,3-dioxolane, 2-(2-(2-chloroethoxy)ethoxy)tetrahydro-2H-pyran, and 2-(chloromethoxy)ethyl benzoate. Procedures to deprotect these cases at the final or intermediate stage are well established (Greene, T. W.; Wuts, P. G. M. Protective Groups in Organic Synthesis, 1999).

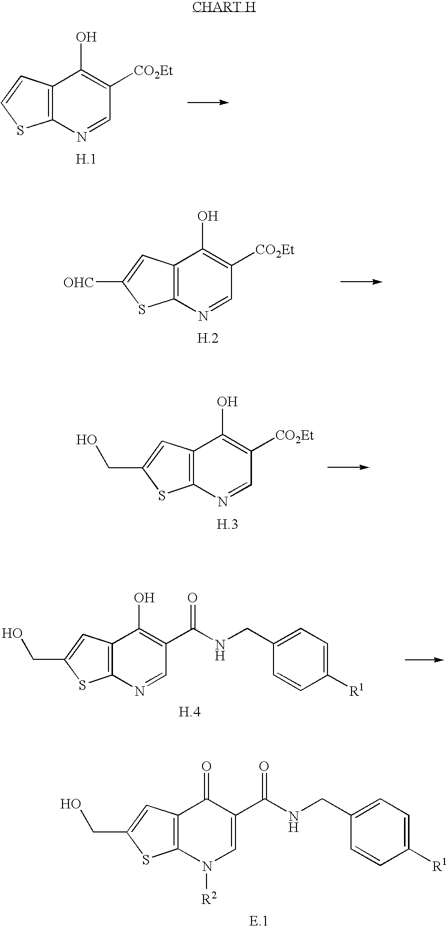

The amine R⁴R⁵C(OH)CH₂NH(R³) in Chart A may be commercially available, can be prepared by procedures know to those skilled in the art, or can be prepared by methods illustrated in Charts I-O. As shown in Chart I, commercially available methylketones 1.1 can be halogenated (X=Cl, Br) to provide the haloketones of the formula 1.2. The resulting haloketones can be reduced to yield the corresponding halohydrins 1.3 employing either achiral (e.g. NaBH₄/CeCl₃) or chiral reduction conditions (e.g. Hamada, T.; Torii, T.; Izawa, K; Noyori, R.; Ikariya, T. Org. Lett. 2002, 4, 4373-4376). The resulting halohydrin is then treated with a primary amine (e.g. methylamine or ethylamine) to afford amins of the formula 1.5. Alternatively, the haloketones can be treated directly with the primary amine (e.g. methylamine or ethylamine) to provide an aminoketone 1.4 which can then be reduced under achiral or chiral reduction conditions (Ohkuma, T.; Ishii, D.; Takeno, H.; Noyori, R. J. Am. Chem. Soc. 2000, 122, 6510-6511; Kawamoto, A.; Wills, M. Tetrahedron: Asymmetry 2000, 11, 3257-3261) to afford compounds of the formula 1.5. In this case, the basic nitrogen may require transient protection (e.g. tert-butylcarbamate) to facilitate the reduction. The precursor N-Boc aminoketones J.2 may be prepared as described in Chart J in which a Weinreb amide derivative (Y=N(CH₃)(OCH₃), prepared by methods well know in the literature, e.g. Sibi, M. Org. Prep. Proc. Int. 1993, 25, 15-40) is reacted with metalated tert-butyl dimethylcarbamate in the presence of tetramethylethylenediamine at low temperature. Other compounds of the formula J.1 which also undergo this reaction include carboxamides wherein Y=4-morpholine and thiol esters (e.g. Y=SPh).

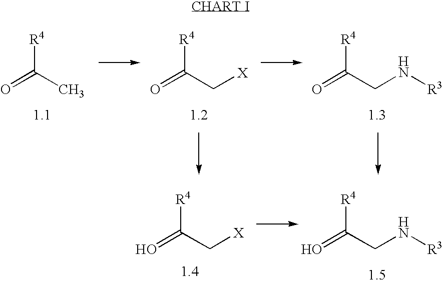

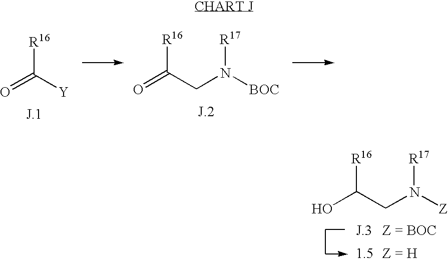

Alternatively, as shown in Chart K specific amines of the formula R⁴R⁵C(OH)CH₂NH(R³) can be prepared from carboxaldehydes K.1 which are commercially available or prepared by methods known to those skilled in the art. Epoxidation of K.1 with a sulfonium ylide (e.g. trimethylsulfonium iodide) affords epoxides of the formula K.2. Treatment of the epoxides with a primary amine (e.g. methylamine or ethylamine) provides compounds of the formula 1.5.

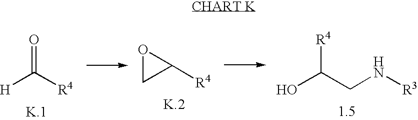

As shown in Chart L, specific amines of the formula R⁴R⁵C(OH)CH₂NH(R³) are also prepared from carbonyl derivatives L.1 by the reaction with metalated tert-butyl dimethylcarbamate in the presence of tetramethylenediamine at low temperature to afford the BOC-protected amino alcohol L.2. Subsequent cleavage under acidic conditions (e.g. trifluoroacetic acid or hydrochloric acid) or oxazolidinone cyclization under basic conditions (e.g. sodium hydride) followed by basic hydrolysis provides compounds of the formula L.3. In cases where R⁵ is hydroxymethyl, 2-hydroxyethyl, or 1-hydroxyethyl, the hydroxyl group is transiently protected using common protecting groups (Greene, T. W.; Wuts, P. G. M. Protective Groups in Organic Synthesis, 1999) and then deprotected either prior to or after coupling as described in Chart A.

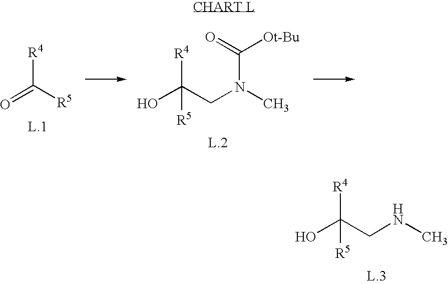

As shown in Chart M, specific amines of the formula R⁴R⁵C(OH)CH₂NH(R³) are also prepared from a protected form of methylaminoacetaldehyde or methylamino-acetaldehyde (e.g. (methyl(trityl)amino)acetaldehyde) (M.1). Treatment of M.1 with a metalated heteroaryl reagent at low temperature affords alcohols of the formula M.2. Subsequent deprotection of the nitrogen protecting group (e.g. in the case of trityl, treatment with an inorganic acid in etheral solution) provides amines of the formula 1.5. It would be understood by those skilled in the art that in some cases transient protection of Lewis basic or acidic functionality present in the R⁴ substituent may be required to facilitate the metal reagent formation and subsequent addition described in Chart L for which procedures are well established (Greene, T. W.; Wuts, P. G. M. Protective Groups in Organic Synthesis, 1999).

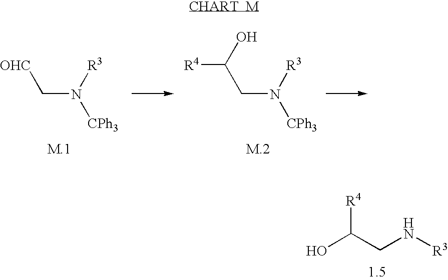

In cases where the R⁵ substituent of the amine R⁴R⁵C(OH)CH₂NH(R³) is methyl or ethyl, the amine may be prepared as described in Chart N. The olefin N.1 is reacted with N-bromosuccinamide in an ether solvent employing a catalytic amount sulfuric acid to afford the bromohydrin N.2. The resulting bromohydrin is then treated with a primary amine (e.g. methylamine or ethylamine) to afford amines of the formula N.3.

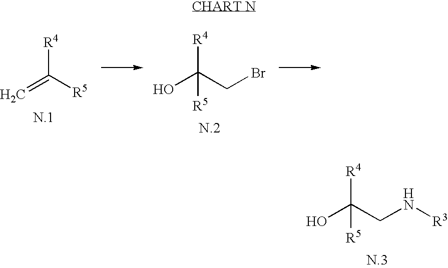

Specific amines of the formula R⁴CH(OH)CH₂NH(CH₃) are also available from primary amines of the formula R⁴CH(OH)CH₂NH₂ according to methods described in Chart O. An amino alcohol of the formula O.1 is treated with dimethyl carbonate and potassium tert-butoxide to afford an oxazolidinone of the formula O.2. The resulting oxazolidinone is subsequently hydrolyzed in the presence of aqueous alkali (e.g. potassium hydroxide) to provide an amino alcohol of the formula O.3.

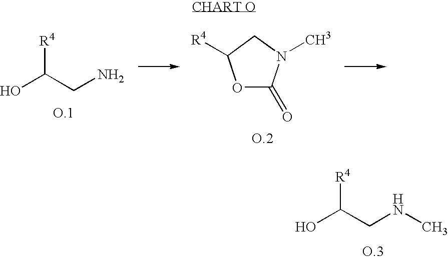

Methods to prepare primary amines of the formula R⁴R⁵C(OH)CH₂NH₂ for use in Chart B are well known to those skilled in the art of organic synthesis (Bergmeier, S. C. Tetrahedron 2000, 56, 2561-2576). In addition to those described herein, representative synthetic examples include 2-amino-1-thien-2-ylethanol (Huebner, C. F.; Diassi, P. A.; Scholz, C. R. J. Org. Chem. 1953, 18, 21); 2-amino-1-(1H-indol-3-yl)ethanol (DeGraw, J. I.; Kennedy, J. G.; Skinner, W. A. J. Heterocyclic Chem. 1966, 3, 9); 2-amino-1-furo[2,3-b]pyridin-2-ylethanol, 2-amino-1-furo[2,3-c]pyridin-2-ylethanol, 2-amino-1-furo[3,2-c]pyridin-2-ylethanol, and 2-amino-1-furo[3,2-b]-pyridin-2-ylethanol (Shiotani, S. J. Heterocyclic Chem. 1993, 30, 1035).

The compounds of Formula (I) may be prepared as single enantiomer or as a mixture of individual enantiomers which includes racemic mixtures. Methods to obtain preferentially a single enantiomer from a mixture of individual enantiomers or a racemic mixture are well known to those ordinarily skilled in the art of organic chemistry. Such methods include but are not limited to preferential crystallization of diastereomeric salts (e.g. tartrate or camphor sulfonate), covalent derivatization by a chiral, non-racemic reagent followed by separation of the resulting diastereomers by common methods (e.g. crystallization, chromatographic separation, or distillation) and chemical reversion to scalemic compound, Simulated Moving Bed technology, or high/medium-pressure liquid chromatography employing a chiral stationary phase (Eliel, E. L. Stereochemistry of Organic Compounds, 1994; Subramanian, G. Chiral Separation Techniques: A Practical Approach, 2001). These techniques may be performed on the final compounds of Formula (I) or on any intermediates to compounds of Formula (I) which bear a sterogenic center. Also, to facilitate separation by any of the methods described above, the compounds of Formula (I) or any intermediates to the compounds of Formula (I) which bear a stereogenic center may be transiently reacted with an achiral reagent, seperated, and then reverted to scalemic compound by standard synthetic techniques.

It will be apparent to those skilled in the art that the described synthetic procedures are merely representative in nature and alternative synthetic processes are known to one of ordinary skill in organic chemistry.

The compounds of the present invention and pharmaceutically acceptable salts thereof are useful as antiviral agents. Thus, these compounds are useful to combat viral infections in mammals. Specifically, these compounds have anti-viral activity against the herpes virus, cytomegalovirus (CMV). These compounds are also active against other herpes viruses, such as the varicella zoster virus, the Epstein-Barr virus, the herpes simplex virus, and the human herpes virus type 8 (HHV-8).

The compounds of the present invention may also be useful for the treatment of several cardiovascular diseases such as atherosclerosis and restenosis. These diseases have been implicated with inflammation of coronary vessel walls resulting from infection or reactivation of herpesviruses.

The compounds of the present invention may also be useful for the treatment of herpesvirus infections in animals, for example, illnesses caused by bovine herpesvirus 1-5 (BHV), ovine herpesvirus 1 and 2, Canine herpesvirus 1, equine herpesvirus 1-8 (EHV), feline herpesvirus 1 (FHV), and pseudorabies virus (PRV).

Pharmaceutical Salts

The compound of formula I may be used in its native form or as a salt. In cases where forming a stable nontoxic salt is desired, administration of the compound as a pharmaceutically acceptable salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, ketoglutarate, and glycerophosphate. Suitable inorganic salts may also be formed, including hydrochloride, hydrobromide, sulfate, nitrate, bicarbonate, and carbonate salts.

Pharmaceutically acceptable salts may be obtained using standard procedures well known in the art, for example by reacting a compound of the invention with a suitable acid affording a physiologically acceptable anion.

Routes of Administration

In therapeutic use for treating, or combating, viral infections in a mammal (i.e. human and animals) a compound of the present invention, its pharmaceutical compositions and other antiviral agents can be administered orally, parenterally, topically, rectally, transmucosally, or intestinally.

Parenteral administrations include indirect injections to generate a systemic effect or direct injections to the afflicted area. Examples of parenteral administrations are subcutaneous, intravenous, intramuscular, intradermal, intrathecal, intraocular, intranasal, intravetricular injections or infusions techniques.

Topical administrations include the treatment of infectious areas or organs readily accessibly by local application, such as, for example, eyes, ears including external and middle ear infections, vaginal, open wound, skins including the surface skin and the underneath dermal structures, or other lower intestinal tract. It also includes transdermal delivery to generate a systemic effect.

The rectal administration includes the form of suppositories.

The transmucosal administration includes nasal aerosol or inhalation applications.

The preferred routes of administration are oral and parenteral.

Composition/Formulation

Pharmaceutical compositions of the present invention may be manufactured by processes well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, solutions, emulsions, gels, syrups, slurries, suspensions and the like, for oral ingestion by a patient. A carrier can be at least one substance which may also function as a diluent, flavoring agent, solubilizer, lubricant, suspending agent, binder, tablet disintegrating agent, and encapsulating agent. Examples of such carriers or excipients include, but are not limited to, magnesium carbonate, magnesium stearate, talc, sugar, lactose, sucrose, pectin, dextrin, mannitol, sorbitol, starches, gelatin, cellulosic materials, low melting wax, cocoa butter or powder, polymers such as polyethylene glycols and other pharmaceutical acceptable materials.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, di- or triglycerides. Stabilizers may be added in these formulations, also.

Liquid form compositions include solutions, suspensions and emulsions. For example, there may be provided solutions of the compounds of this invention dissolved in water and water-propylene glycol and water-polyethylene glycol systems, optionally containing suitable conventional coloring agents, flavoring agents, stabilizers and thickening agents.

The compounds may also be formulated for parenteral administration, e.g., by injection, bolus injection or continuous infusion. Formulations for parenteral administration may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

For injection, the compounds of the invention may be formulated in aqueous solution, preferably in physiologically compatible buffers or physiological saline buffer. Suitable buffering agents include trisodium orthophosphate, sodium bicarbonate, sodium citrate, N-methylglucamine, L(+)-lysine and L(+)-arginine.

Parenteral administrations also include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use. For suppository administration, the compounds may also be formulated by mixing the agent with a suitable non-irritating excipient which is solid at room temperature but liquid at rectal temperature and therefore will melt in the rectum to release the drug. Such materials include cocoa butter, beeswax and other glycerides.

For administration by inhalation, compounds of the present invention can be conveniently delivered through an aerosol spray in the form of solution, dry powder, or suspensions. The aerosol may use a pressurized pack or a nebulizer and a suitable propellant. In the case of a pressurized aerosol, the dosage unit may be controlled by providing a valve to deliver a metered amount. Capsules and cartridges of, for example, gelatin for use in an inhaler may be formulated containing a power base such as lactose or starch.

For topical applications, the pharmaceutical composition may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion such as suspensions, emulsion, or cream containing the active components suspended or dissolved in one or more pharmaceutically acceptable carriers. Suitable carriers include, but are not limited to, mineral oil, sorbitan monostearate, polysorbate 60, cetyl esters wax, ceteary alcohol, 2-octyldodecanol, benzyl alcohol and water.

For ophthalmic and otitis uses, the pharmaceutical compositions may be formulated as micronized suspensions in isotonic, pH adjusted sterile saline, or preferably, as solutions in isotonic, pH adjusted sterile saline, either with or without a preservative such as a benzylalkonium chloride. Alternatively, for ophthalmic uses, the pharmaceutical compositions may be formulated in an ointment such as petrolatum.

In addition to the formulations described previously, the compounds may also be formulated as depot preparations. Such long acting formulations may be in the form of implants. A compound of this invention may be formulated for this route of administration with suitable polymers, hydrophobic materials, or as a sparing soluble derivative such as, without limitation, a sparingly soluble salt.

Additionally, the compounds may be delivered using a sustained-release system. Various sustained-release materials have been established and are well known by those skilled in the art. Sustained-release capsules may, depending on their chemical nature, release the compounds for 24 hours or for up to several days.

Dosage

Pharmaceutical compositions suitable for use in the present invention include compositions wherein the active ingredients are contained in an amount sufficient to achieve the intended purpose, i.e., the treatment or prevention of infectious diseases. More specifically, a therapeutically effective amount means an amount of compound effective to prevent, alleviate or ameliorate symptoms of disease or prolong the survival of the subject being treated.

The quantity of active component, that is the compound of this invention, in the pharmaceutical composition and unit dosage form thereof may be varied or adjusted widely depending upon the manner of administration, the potency of the particular compound and the desired concentration. Determination of a therapeutically effective amount is well within the capability of those skilled in the art. Generally, the quantity of active component will range between 0.5% to 90% by weight of the composition.

Generally, an antiviral effective amount of dosage of active component will be in the range of about 0.1 to about 400 mg/kg of body weight/day, more preferably about 1.0 to about 50 mg/kg of body weight/day. It is to be understood that the dosages may vary depending upon the requirements of each subject and the severity of the viral infection being treated.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation. If desired, the daily dose may also be divided into multiple doses for administration, e.g., two to four times per day.

In cases of local administration or selective uptake, the effective local concentration of the drug may not be related to plasma concentration and other procedures know in the art may be used to determine the desired dosage amount.

### Biological Data

While many of the compounds of the present invention have shown activity against the CMV polymerase, these compounds may be active against the cytomegalovirus by this or other mechanisms of action. Thus, the description below of these compounds' activity against the CMV polymerase is not meant to limit the present invention to a specific mechanism of action.

The compounds of the present invention have shown activity in one or more of the assays described below. All of these assays are indicative of a compound's activity and thus of its use as an anti-viral agent.

The HCMV polymerase assay is performed using a scintillation proximity assay (SPA) as described in several references, such as N. D. Cook, et al., Pharmaceutical Manufacturing International, pages 49-53 (1992); KY Takeuchi, Laboratory Practice, September issue (1992); U.S. Pat. No. 4,568,649 (1986); which are incorporated by reference herein. Reactions are performed in 96-well plates. The assay is conducted in 100 yl volume with 5.4 mM HEPES (pH 7.5), 11.7 mM KCl, 4.5 mM MgCl₂, 0.36 mg/ml BSA, and 90 nM ³H-dTTP. Assays are run with and without CHAPS, (3-[(3-Cholamidopropyl)-dimethylammonio]-1-propane-sulfonate) at a final concentration of 2 mM. HCMV polymerase is diluted in enzyme dilution buffer containing 50% glycerol, 250 mM NaCl, 10 mM HEPES (pH 7.5), 100 μg/ml BSA, and 0.01% sodium azide. The HCMV polymerase, which is expressed in recombinant baculovirus-infected SF-9 cells and purified according to literature procedures, is added at 10% (or 10 μl) of the final reaction volume, i.e., 100 μl. Compounds are diluted in 50% DMSO and 10 μl are added to each well. Control wells contain an equivalent concentration of DMSO. Unless noted otherwise, reactions are initiated via the addition of 6 nM biotinylated poly(dA)-oligo(dT) template/primer to reaction mixtures containing the enzyme, substrate, and compounds of interest. Plates are incubated in a 25° C. or 37° C. H₂O bath and terminated via the addition of 40 μl/reaction of 0.5 M EDTA (pH 8) per well. Reactions are terminated within the time-frame during which substrate incorporation is linear and varied depending upon the enzyme and conditions used, i.e., 30 min. for HCMV polymerase. Ten (10) μl of streptavidin-SPA beads (20 mg/ml in PBS/10% glycerol) are added following termination of the reaction. Plates are incubated 10 min. at 37° C., then equilibrated to room temperature, and counted on a Packard Topcount. Linear regressions are performed and IC₅₀'s are calculated using computer software.

A modified version of the above HCMV polymerase assay is performed as described above, but with the following changes: Compounds are diluted in 100% DMSO until final dilution into assay buffer. In the previous assay, compounds are diluted in 50% DMSO. 4.5 mM Dithiothreitol (DTT) is added to the polymerase buffer. Also, a different lot of CMV polymerase is used, which appears to be more active resulting in a more rapid polymerase reaction.

Results of the testing of compounds of the present invention in this assay are shown in Tables 1 below.

All results are listed as Polymerase IC₅₀ (EM) values. In Table 1, the term “n.d.” refers to activity data not determined.

TABLE 1

| | Polymerase IC₅₀ (μM) | | |
|---|---|---|---|
| Example | HCMV | HSV | VZV |
| 1 | 0.08 | 0.22 | 0.06 |
| 2 | 0.04 | 0.09 | 0.03 |
| 3 | 0.07 | 0.26 | 0.13 |
| 4 | 0.25 | 0.84 | 0.39 |
| 5 | 0.01 | 0.03 | 0.01 |
| 6 | 0.17 | 0.47 | 0.15 |
| 7 | 0.30 | 0.98 | 0.33 |
| 8 | 0.26 | 0.85 | 0.32 |
| 9 | 1.06 | nd | nd |
| 10 | 0.45 | 1.97 | 0.44 |
| 11 | 0.09 | 0.20 | 0.09 |
| 12 | 0.28 | nd | nd |
| 13 | 0.17 | 1.92 | 0.44 |
| 14 | 0.10 | 0.53 | 0.12 |
| 15 | 0.24 | nd | nd |
| 16 | 0.20 | 0.45 | 0.19 |
| 17 | 0.12 | 0.39 | 0.11 |
| 18 | 0.35 | 0.91 | 0.37 |
| 19 | 0.21 | 0.78 | 0.29 |
| 20 | 0.22 | 0.46 | 0.20 |
| 21 | 0.35 | nd | nd |
| 22 | 0.09 | 0.17 | 0.08 |
| 23 | 0.26 | nd | nd |
| 24 | 0.31 | nd | nd |
| 25 | 1.80 | nd | nd |
| 26 | 0.15 | 0.54 | 0.33 |
| 27 | 0.36 | 0.59 | 0.28 |
| 28 | 0.30 | nd | nd |
| 29 | 0.60 | nd | nd |
| 30 | 0.14 | 0.62 | 0.20 |
| 31 | 0.07 | 0.12 | 0.06 |
| 32 | 0.18 | 0.74 | 0.22 |
| 33 | 0.02 | 0.08 | nd |
| 34 | 0.24 | 1.08 | 0.34 |
| 35 | 0.56 | nd | nd |
| 36 | 0.83 | nd | nd |

### EXAMPLES

Preparation 1.

N-(4-Chlorobenzyl)-2-(chloromethyl)-7-methyl-4-oxo-4,7-dihydrothieno[2,3-b]-pyridine-5-carboxamide.

Procedure A. N-(4-Chlorobenzyl)-2-(hydroxymethyl)-7-methyl-4-oxo-4,7-dihydro-thieno[2,3-b]pyridine-5-carboxamide (3.00 g, prepared as described in U.S. Pat. No. 6,239,142) is dissolved in DMF (150 mL). DMAP (0.150 g), 2,4,6-collidine (2.73 mL), and methanesulfonyl chloride (1.60 mL) are added, and the reaction mixture is stirred at room temperature for 18 h. The reaction mixture is poured into water (300 mL). The resulting pale yellow solid is filtered off and triturated with acetonitrile to yield 2.75 g of the title compound. Physical characteristics. M.p. 250-256° C. (dec); ¹H NMR (400 MHz, DMSO-d₆) δ 10.48, 8.74, 7.58, 7.41-7.33, 5.16, 4.55, 3.97; ¹³C NMR (DMSO-d₆) δ 172.5, 164.5, 151.8, 146.4, 138.9, 135.7, 131.7, 130.5, 129.5, 128.7, 124.0, 115.0, 43.4, 41.8, 41.1; MS (EI) m/z 380 (M⁺); HRMS (FAB) m/z 381.0255 (M+H)⁺. Anal. Found: C, 53.34; H, 3.70; N, 7.30; Cl, 17.91; S, 8.51.

Procedure B. A 25 mL round-bottomed flask is charged with N-(4-chlorobenzyl)-7-methyl-2-(morpholin-4-ylmethyl)-4-oxo-4,7-dihydrothieno[2,3-b]pyridine-5-carboxamide (1.00 g, prepared as described in U.S. Pat. No. 6,239,142) and chloroform (10 ml) via syringe. Ethyl chloroformate (0.55 mL) is added via syringe with stirring under nitrogen. The slurry is heated to reflux overnight. Anhydrous diethyl ether (10 ml) is added to the slurry with stirring under nitrogen. The solid is filtered and washed with diethyl ether (3×10 mL). The product is dried in the vacuum oven at 40° C. to afford 0.93 g of the title compound as colorless crystals. Physical characteristics. ¹H NMR (400 MHz, TFA-d) δ 9.09, 7.69, 7.22, 4.81, 4.62, 4.27; ¹³C NMR (100 MHz, TFA-d) δ 167.6, 166.6, 156.3, 145.2, 143.6, 134.9, 133.3, 129.1, 129.0, 127.4, 119.6, 109.9, 45.2, 44.0, 38.0. Anal. Found: C, 53.44; H, 3.66; N, 7.35; Cl, 18.29.

Preparation 2.

N-(4-Chlorobenzyl)-7-((2,2-dimethyl-1,3-dioxolan-4-yl)methyl)-2-(hydroxy-methyl)-4-oxo-4,7-dihydrothieno[2,3-b]pyridine-5-carboxamide.

Cesium carbonate (3.91 g) is added to a solution of N-(4-chlorobenzyl)-4-hydroxy-2-(hydroxymethyl)thieno[2,3-b]pyridine-5-carboxamide (3.49 g, prepared as described in U.S. Pat. No. 6,239,142) and 4-(bromomethyl)-2,2-dimethyl-1,3-dioxolane (1.95 g) in DMF (20 mL). The reaction mixture is stirred at 100° C. for 17 h. The solvent is evaporated and the redisue is dissolved in 10% CH₃OH in CH₂Cl₂. The mixture is washed with water and the organic layer is dried (MgSO₄), filtered, and concentrated. The crude product is crystallized from EtOAc to afford 2.7 g of the title compound as a white solid. Physical characteristics. ¹H NMR (400 MHz, DMSO-d₆) δ 10.53, 8.70, 7.40, 7.34, 7.28, 5.79, 4.69, 4.53, 4.50, 4.30, 4.14, 3.77, 1.34, 1.23; MS (EI) m/z 462 (M⁺); HRMS (FAB) m/z 463.1087 (M+H)⁺. Anal. Found: C, 57.07; H, 5.01; N, 6.05.

Preparation 3.

2-(Chloromethyl)-N-((4-chlorophenyl)methyl)-7-((2,2-dimethyl-1,3-dioxolan-4-yl)methyl]-4,7-dihydro-4-oxothieno[2,3-b]pyridine-5-carboxamide.

2,4,6-Collidine (1.78 mL) and a few crystals of 4-N,N-dimethylaminopyridine are added to a solution of N-(4-chlorobenzyl)-7-((2,2-dimethyl-1,3-dioxolan-4-yl)methyl)-2-(hydroxymethyl)-4-oxo-4,7-dihydrothieno[2,3-b]pyridine-5-carboxamide (Preparation 2, 2.33 g) in DMF (15 mL). Methanesulfonyl chloride (0.93 mL) is added dropwise and the reaction is stirred at room temperature for 4 hours. The solvent is evaporated and the residue is dissolved in 10% MeOH in CH₂Cl₂. The mixture is washed with water, dried with MgSO₄, filtered, and concentrated. The residue is chromatographed over silica gel with 5% MeOH in CH₂Cl₂ and the product is crystallized from EtOAc, filtered and washed with ether to afford 1.73 g of the title compound as white crystals. Physical characteristics: ¹H NMR (400 MHz, DMSO-d₆) δ 10.42, 8.73, 7.55, 7.39, 7.34, 5.15, 4.54, 4.51, 4.30, 4.14, 3.77, 1.34, 1.23; HRMS (FAB) m/z 481.0758 (M+H)⁺. Anal. Found: C, 55.18; H, 4.76; N, 5.66.

Preparation 4.

N-(4-Chlorobenzyl)-2-(hydroxymethyl)-4-oxo-7-(3-(tetrahydro-2H-pyran-2-yloxy)propyl)-4,7-dihydrothieno[2,3-b]pyridine-5-carboxamide.

Cesium carbonate (5.54 g) is added to a solution of N-(4-chlorobenzyl)-4-hydroxy-2-(hydroxymethyl)thieno[2,3-b]pyridine-5-carboxamide (5.23 g, prepared as described in U.S. Pat. No. 6,239,142) and 2-(3-iodopropoxy)tetrahydro-2H-pyran (4.32 g, prepared by mixing equal molar amounts of 2-iodoethanol and 3,4-Dihydro-2H-pyran) in DMF (20 mL). The mixture is heated at 60° C. for 4 hours. The solvent is evaporated and the residue is dissolved in 10% MeOH in CH₂Cl₂. The mixture is washed with water and the organic layer is dried (MgSO₄), filtered, and concentrated. The crude product is purified by chromatographed over silica gel with 5% MeOH in CH₂Cl₂ and recrystallization from EtOAc to afford 4.82 g of the title compound as white crystals. Physical characteristics. ¹H NMR (400 MHz, DMSO-d₆) δ 10.55, 8.71, 7.39, 7.33, 7.29, 5.79, 4.70, 4.53, 4.49, 4.38, 3.68, 3.37, 2.11, 1.63, 1.53, 1.40; MS (EI) m/z 490 (M⁺); Anal. Found: C, 58.74; H, 5.66; N, 5.61.

Preparation 5.

N-(4-Chlorobenzyl)-2-(chloromethyl)-4-oxo-7-(3-(tetrahydro-2H-pyran-2-yloxy)-propyl)-4,7-dihydrothieno[2,3-b]pyridine-5-carboxamide.

2,4,6-Collidine (2.51 mL) and a few crystals of 4-N,N-dimethylaminopyridine is added to a solution of N-(4-chlorobenzyl)-2-(hydroxymethyl)-4-oxo-7-(3-(tetrahydro-2H-pyran-2-yloxy)propyl)-4,7-dihydrothieno[2,3-b]pyridine-5-carboxamide (Preparation 4, 4.0 g) in DMF (20 mL). Methanesulfonyl chloride (1.38 mL) is added dropwise and the reaction is stirred at 60° C. for 5 hours. The solvent is evaporated and the residue dissolved in 10% MeOH in CH₂Cl₂. The mixture is washed with water and the organic layer is dried (MgSO₄), filtered, and concentrated. The residue is chromatographed over silica gel with 5% MeOH in CH₂Cl₂. The crude product is crystallized from EtOAc, filtered, and washed with ether to afford 2.35 g of the title compound as white crystals. Physical characteristics. ¹H NMR (400 MHz, DMSO-d₆) δ 10.43, 8.74, 7.56, 7.39, 7.34, 5.15, 4.54, 4.38, 3.70, 3.38, 2.11, 1.61, 1.51, 1.38; MS (EI) m/z 508 (M⁺); HRMS (FAB) m/z 509.1064 (M+H)⁺. Anal. Found: C, 56.00; H15.11; N, 5.56.

Preparation 6.

N-(4-Chlorobenzyl)-2-(hydroxymethyl)-4-oxo-7-(2-(tetrahydro-2H-pyran-2-yloxy)ethyl)-4,7-dihydrothieno[2,3-b]pyridine-5-carboxamide.

Cesium carbonate (3.91 g) is added to a solution of N-(4-chlorobenzyl)-4-hydroxy-2-(hydroxymethyl)thieno[2,3-b]pyridine-5-carboxamide (3.49 g, prepared as described in U.S. Pat. No. 6,239,142) and 2-(2-iodoethoxy)tetrahydro-2H-pyran (2.56 g, prepared by mixing equal molar amounts of 2-iodoethanol and 3,4-dihydro-2H-pyran) in DMF (20 mL). The reaction mixture is stirred at 100° C. for 17 hours. The solvent is evaporated and the residue is dissolved in 10% CH₃OH in CH₂Cl₂. The mixture is washed with water and the organic layer is dried (MgSO₄), filtered, concentrated. The crude product is crystallized from EtOAc to afford 3.8 g of the title compound as a white solid. Physical characteristics. ¹H NMR (400 MHz, DMSO-d₆) δ 10.59, 8.71, 7.39, 7.38, 7.29, 5.79, 4.69, 4.58, 4.54, 4.48, 3.96, 3.78, 3.30, 1.54, 1.39, 1.29; MS (EI) m/z 476 (M); HRMS (FAB) m/z 477.1245 (M+H)⁺.

Preparation 7.

N-(4-Chlorobenzyl)-2-(chloromethyl)-4-oxo-7-(2-(tetrahydro-2H-pyran-2-yloxy)ethyl)]-4,7-dihydrothieno[2,3-b]pyridine-5-carboxamide.

2,4,6-Collidine (2.9 mL) and a few crystals of 4-N,N-dimethylaminopyridine is added to a solution of N-(4-chlorobenzyl)-2-(hydroxymethyl)-4-oxo-7-(2-(tetrahydro-2H-pyran-2-yloxy)ethyl)-4,7-dihydrothieno[2,3-b]pyridine-5-carboxamide (Preparation 6, 3.5 g) in DMF (20 mL). Methanesulfonyl chloride (1.7 mL) is added dropwise and the reaction mixture is stirred at room temperature for 72 h. The reaction mixture is poured into water (100 mL) and filtered. The filtrate is extracted with 10% MeOH in CH₂Cl₂. The organic layer is dried (MgSO₄), filtered, and concentrated to afford 2.8 g of the title compound as a white solid. Physical characteristics. ¹H NMR (400 MHz, DMSO-d₆) δ 10.43, 8.75, 7.55, 7.38, 7.33, 5.14, 4.59, 4.53, 4.49, 3.96, 3.79, 3.29, 1.52, 1.38, 1.28; MS (EI) m/z 494 (M⁺); HRMS (FAB) m/z 495.0904 (M+H)⁺.

Preparation 8.

N-(4-Chlorobenzyl)-2-(chloromethyl)-7-(2-hydroxyethyl)]-4-oxo-4,7-dihydro-thieno [2,3-b]pyridine-5-carboxamide.

2,4,6-Collidine (2.9 mL) and a few crystals of 4-N,N-dimethylaminopyridine is added to a solution of N-(4-chlorobenzyl)-2-(hydroxymethyl)-4-oxo-7-(2-(tetrahydro-2H-pyran-2-yloxy)ethyl)-4,7-dihydrothieno[2,3-b]pyridine-5-carboxamide (Preparation 6, 3.5 g) in DMF (20 mL). Methanesulfonyl chloride (1.7 mL) is added dropwise and the reaction mixture is stirred at room temperature for 72 h. The reaction mixture is poured into water (100 mL) and filtered. The solid is recrystallized from acetonitrile to afford 1.27 g of the title compound as a white solid. Physical characteristics. ¹H NMR (400 MHz, DMSO-d₆) δ 10.47, 8.67, 7.55, 7.40, 7.34, 5.15, 5.14, 4.54, 3.34, 2.51; MS (EI) m/z 410 (M⁺); HRMS (FAB) m/z 411.0332 (M+H)⁺. Anal. Found: C, 52.27; H, 4.05; N, 6.93.

Preparation 9.

N-(4-Chlorobenzyl)-7-ethyl-2-(hydroxymethyl)-4-oxo-4,7-dihydrothieno[2,3-b]-pyridine-5-carboxamide.

Potassium carbonate (0.87 g) and iodoethane (0.5 mL) are added to a solution of N-(4-chlorobenzyl)-4-hydroxy-2-(hydroxymethyl)thieno[2,3-b]pyridine-5-carboxamide (2.0 g, prepared as described in U.S. Pat. No. 6,239,142) in anhydrous DMF (60 mL). The reaction mixture is stirred at room temperature for 18 h. The mixture is diluted with water (150 mL) and filtered. The resulting white powder is washed with water (15 mL) followed by diethyl ether (15 mL) and dried in a vacuum oven to afford 1.64 g of the title compound as a white solid. Physical characteristics. M.p. 169-172° C.; ¹H NMR (300 MHz, DMSO-d₆) δ 10.65, 8.74, 7.37, 7.29, 5.81, 4.70, 4.54, 4.32, 1.44. HRMS (FAB) m/z 377.0720 (M+H)⁺. Anal. Found: C, 56.87; H, 4.77; N, 7.38; Cl, 9.35; S, 8.44.

Preparation 10.

N-(4-Chlorobenzyl)-2-(chloromethyl)-7-ethyl-4-oxo-4,7-dihydrothieno[2,3-b]-pyridine-5-carboxamide.

4-N,N-Dimethylaminopyridine (80 mg), 2,4,6-collidine (1.41 mL), and methanesulfonyl chloride (0.83 mL) are added to a solution of N-(4-chlorobenzyl)-7-ethyl-2-(hydroxymethyl)-4-oxo-4,7-dihydrothieno[2,3-b]pyridine-5-carboxamide (Preparation 9, 1.61 g) in anhydrous DMF (80 mL). The reaction mixture is stirred at room temperature for 24 h. The mixture is diluted with water (150 mL) and filtered. The resulting white powder is recrystallized from acetonitrile and dried in a vacuum oven to afford 1.4 g of the title compound as a white solid. Physical characteristics. M.p. 199-200° C.; ¹H NMR (300 MHz, DMSO-d6) δ 10.45, 8.77, 7.57, 7.38, 5.15, 4.54, 4.32, 1.44. Anal. Found: C, 54.53; H, 3.94; N, 7.03; Cl, 17.57; S, 8.09.

Preparation 11.

N-(4-Chlorobenzyl)-7-propyl-2-(hydroxymethyl)-4-oxo-4,7-dihydrothieno[2,3-b]-pyridine-5-carboxamide.

Potassium carbonate (0.91 g) and 1-iodopropane (0.64 mL) are added to a solution of N-(4-chlorobenzyl)-4-hydroxy-2-(hydroxymethyl)thieno[2,3-b]pyridine-5-carboxamide (2.0 g, prepared as described in U.S. Pat. No. 6,239,142) in anhydrous DMF (60 mL). The reaction mixture is stirred at room temperature for 4 h. The mixture is diluted with water (150 mL) and filtered. The resulting white powder is washed with water (15 mL) followed by diethyl ether (15 mL) and dried in a vacuum oven to afford 1.73 g of the title compound as a white solid. Physical characteristics. M.p. 174-175° C. ¹H NMR (300 MHz, DMSO-d6) δ 10.62, 8.72, 7.38, 7.29, 5.80, 4.69, 4.55, 4.27, 1.87, 0.89; Anal. Found: C, 58.20; H. 4.96; N, 7.13; Cl, 8.98; S, 8.16.

Preparation 12.

N-(4-Chlorobenzyl)-2-(chloromethyl)-7-propyl-4-oxo-4,7-dihydrothieno[2,3-b]-pyridine-5-carboxamide.

4-N,N-Dimethylaminopyridine (80 mg), 2,4,6-collidine (1.39 mL), and methanesulfonyl chloride (0.81 mL) are added to a solution of N-(4-chlorobenzyl)-7-propyl-2-(hydroxymethyl)-4-oxo-4,7-dihydrothieno[2,3-b]pyridine-5-carboxamide (Preparation 11, 1.63 g) in anhydrous DMF (80 mL). The reaction mixture is stirred at room temperature for 24 h. The mixture is diluted with water (150 mL) and filtered. The resulting light yellow powder is recrystallized from acetonitrile and dried in a vacuum oven to afford 1.4 g of the title compound as a light yellow solid. Physical characteristics. M.p. 186.5-188° C.; ¹H NMR (300 MHz, DMSO-d6) δ 10.45, 8.75, 7.56, 7.39, 5.15, 4.54, 4.27, 1.85, 0.91. Anal. Found: C, 55.76; H, 4.59; N, 6.95; Cl, 16.88; S, 7.80.

Preparation 13.

N-(4-Chlorobenzyl)-2-(hydroxymethyl)-7-(2-methoxyethyl)-4-oxo-4,7-dihydro-thieno[2,3-b]pyridine-5-carboxamide.

Potassium carbonate (5.0 g) and bromoethylmethyl ether (5.0 g) are added to a solution of N-(4-chlorobenzyl)-4-hydroxy-2-(hydroxymethyl)thieno[2,3-b]pyridine-5-carboxamide (11.4 g, prepared as described in U.S. Pat. No. 6,239,142) in anhydrous DMF (350 mL). The reaction mixture is stirred at room temperature for 18 h. The mixture is diluted with water (600 mL) and filtered. The resulting white powder is dried in a vacuum oven to afford 8.44 g of the title compound as a white solid. Physical characteristics. M.p. 193° C.; ¹H NMR (300 MHz, DMSO-d6) δ 10.58, 8.65, 7.37, 7.29, 5.82, 4.70, 4.54, 4.47, 3.76, 3.24. HRMS (FAB) m/z 407.0836 (M+H)⁺. Anal. Found: C, 55.81; H, 4.71; N, 6.90; Cl, 8.58; S, 7.81.

Preparation 14.

N-(4-Chlorobenzyl)-2-(chloromethyl)-7-(2-methoxyethyl)-4-oxo-4,7-dihydro-thieno[2,3-b]pyridine-5-carboxamide.

4-N,N-Dimethylaminopyridine (360 mg), 2,4,6-collidine (6.5 mL), and methanesulfonyl chloride (3.8 mL) are added to a solution of N-(4-chlorobenzyl)-2-(hydroxymethyl)-7-(2-methoxyethyl)-4-oxo-4,7-dihydrothieno[2,3-b]pyridine-5-carboxamide (Preparation 13, 8.0 g) in anhydrous DMF (360 mL). The reaction mixture is stirred at room temperature for 18 h. The mixture is diluted with water (600 mL) and filtered. The resulting off-white powder is dried in a vacuum oven to afford 7.03 g of the title compound as an off-white solid. Physical characteristics. M.p. 192-193° C.; ¹H NMR (300 MHz, DMSO-d6) δ 10.48, 8.67, 7.55, 7.37, 5.14, 4.53, 4.46, 3.74, 3.24; HRMS (FAB) m/z 425.0480 (M+H)⁺. Anal. Found: C, 53.38; H, 4.37; N, 6.66; Cl, 15.77; S, 7.69.

Preparation 15.

2-Furoyl bromide.

Bromine (6.5 mL) is added dropwise over 1 h to a solution of 2-acetylfuran (11.0 g) in dioxane/Et₂O (1/2, 60 mL) at 0° C. (internal). The reaction mixture is then allowed to warm to room temperature and is stirred for 2 h. A saturated ammonium chloride solution (70 mL) is added. The organic layer is removed, and the aqueous layer is extracted with diethyl ether (2×50 mL). The combined organic layers are dried (MgSO₄), filtered, and concentrated in vacuo. The resulting brown solid is purified by column chromatography (hexanes/CH₂Cl₂, 70/30) to yield 7.996 g of the title compound as a yellow oil. Physical characteristics. ¹H NMR (400 MHz, DMSO-d₆) δ 8.09, 7.66-7.64, 6.79-6.77, 4.65.

Preparation 16.

rac-1-(2-Furyl)-2-(methylamino)ethanol.

A solution of 2-furoyl bromide (Preparation 15, 7.50 g) in methanol (40 mL) is added dropwise to a 2.0 M solution of methylamine in methanol (198 mL) at 0° C. (internal). The reaction mixture is stirred at 0° C. for 30 min. A solution of sodium borohydride (2.25 g) in water (40 mL) is then added dropwise. The reaction mixture is stirred at 0° C. for 30 min and then quenched with a 2 N HCl solution (to pH 3-4). The reaction mixture is concentrated in vacuo to remove methanol and is then poured into cold EtOAc (200 mL)/2 N NaOH (100 mL). The organic layer is removed. The aqueous layer is adjusted to pH 12 with a 2 N NaOH solution and extracted with EtOAc (3×200 mL). The combined organic layers are dried (MgSO₄), filtered, and concentrated in vacuo. The resulting brown oil is purified by column chromatography (CHCl₃/methanol, 95/5; CHCl₃/methanol/NH₄OH, 90/10/1) to yield 2.06 g of the title compound as a brown oil. Physical characteristics. ¹H NMR (400 MHz, DMSO-d₆) δ 7.56, 6.39-6.37, 6.26-6.25, 5.15, 4.62-4.58, 2.77-2.66, 2.33; MS (ESI+) m/z 142 (M+H)⁺.

Preparation 17.

(1R)-1-(2-Furyl)-2-(methylamino)ethanol.

A 250 mL round-bottomed flask equipped with an overhead stirrer, reflux condensor, thermocouple and an addition funnel is charged with (R)-2-amino-1-(2-furyl)ethanol (10 g) and potassium t-butoxide (10.6 g). Anhydrous DMF is charged at such a rate as to keep the temperature less than 50° C. The reaction is heated to 80° C. (internal temp), the addition funnel is charged with dimethyl carbonate (50 mL), and the liquid is added to the reaction drop-wise. Once addition of dimethyl carbonate is complete, the temperature is raised to reflux (about 100° C.), and maintained for approximately 12 h. The reaction mixture is cooled to less than 60° C., poured into water (100 mL) and extracted with isopropyl acetate (100 mL). The layers are separated, and the water layer is extracted with additional isopropyl acetate (2×100 mL). The combined organic layers are washed with water (100 mL) and dried over sodium sulfate and magnesol for 10 min. The solids are removed via vacuum filtration, and the organic layers are concentrated in vacuo. The resulting oil is crystallized from MTBE (2 mL/g) to provide 10.25 g of (5R)-5-(2-furyl)-3-methyl-1,3-oxazolidin-2-one. Physical characteristics. ¹H NMR (CDCl₃, 400 MHz) δ 7.46, 6.49, 6.39, 5.47, 3.78, 2.97.

A round-bottomed flask equipped with an overhead stirrer, reflux condensor and nitrogen inlet is charged with (5R)-5-(2-furyl)-3-methyl-1,3-oxazolidin-2-one (47.1 g). A 1 M solution of KOH (987 mL) is added and the resulting solution is heated at 50° C. When complete, the flask is charged with NaCl (310 g) and MTBE (470 mL). The aqueous layer is seperated and further extracted twice with a solution of MTBE (470 mL) and CH₂Cl₂ (23 mL). The combined organic layers are dried (MgSO₄), filtered, and concentrated to afford 38.0 g of the title compound. Physical characteristics. ¹H NMR (DMSO, 400 MHz) δ 7.52, 6.36, 6.24, 4.60, 2.71, 2.28; ¹³C NMR (DMSO, 100 MHz) δ 157.0, 141.6, 110.1, 105.6, 65.2, 56.2, 35.9; [α]²²_{D}=+34° (EtOH, c=1.0).

Preparation 18.

2-Bromo-1-(5-methyl-2-furyl)ethanone.

Bromine (5.1 mL) is added dropwise over 1 h to a solution of 2-acetyl-5-methylfuran (11.0 g) in dioxane/Et₂O (1/2, 60 mL) at 0° C. (internal). The reaction mixture is stirred at 0° C. for 30 min and then allowed to warm to room temperature and is stirred for 18 h. The reaction mixture is cooled to 0° C. (internal), and additional bromine (1.53 mL) is added dropwise. The reaction mixture is allowed to warm to room temperature and is stirred for 1 h. A saturated ammonium chloride solution (100 mL) is added. The organic layer is removed, and the aqueous layer is extracted with Et₂O (2×100 mL). The combined organic layers are dried (MgSO₄), filtered, and concentrated in vacuo. The resulting brown solid is purified via column chromatography (hexanes/CH₂Cl₂, 70/30) to yield a yellow solid which is recrystallized from EtOAc/hexanes to yield 8.571 g of the title compound as a pale yellow solid. Physical characteristics. M.p. 60-63° C.; ¹H NMR (400 MHz, DMSO-d₆) δ 7.60, 6.44, 4.58, 2.41.

Preparation 19.

2-(Methylamino)-1-(5-methyl-2-furyl)ethanol.

A solution of 2-bromo-1-(5-methyl-2-furyl)ethanone (Preparation 18, 8.00 g) in methanol (100 mL) is added dropwise to a 2.0 M solution of methylamine in methanol (197 mL) at 0° C. (internal). The reaction mixture is stirred at 0° C. for 30 min. A solution of sodium borohydride (2.23 g) in water (40 mL) is then added dropwise. The reaction mixture is stirred at 0° C. for 1.5 h and then quenched with a 2 N HCl solution (to pH 3-4). The reaction mixture is concentrated in vacuo to remove methanol and then poured into cold EtOAc (200 mL)/2 N NaOH (100 mL). The organic layer is removed. The aqueous layer is adjusted to pH 12 with a 2 N NaOH solution and extracted with EtOAc (3×200 mL). The combined organic layers are dried (MgSO₄), filtered, and concentrated in vacuo. The resulting yellow oil is purified via column chromatography (CHCl₃/methanol, 95/5, 90/10; CHCl₃/methanol/NH₄OH, 90/10/1). The resulting yellow oil is crystallized from diethyl ether to yield 1.88 g of the title compound as a yellow solid. Physical characteristics. M.p. 40-45° C.; ¹H NMR (400 MHz, DMSO-d₆) δ 6.11, 5.97-5.96, 5.05, 4.54-4.51, 2.72-2.65, 2.29, 2.22; MS (ESI+) m/z 156 (M+H)⁺.

Preparation 20.

1-(3-Furyl)-2-(methylamino)ethanol.

Trimethylsulfonium iodide (20.4 g) and 3-furaldehyde (8.65 mL) are added to potassium hydroxide (11.2 g) and water (0.45 mL) in acetonitrile (150 mL). The reaction mixture is heated to 60° C. for 2.5 h. The reaction mixture is allowed to cool to room temperature. The precipitate is filtered off, and the filtrate is concentrated in vacuo. The resulting crude epoxide (10.747 g) is dissolved in methanol (50 mL) and added to a 2.0 M solution of methylamine in methanol (100 mL). The reaction mixture is stirred at room temperature for 3 d and then heated to reflux for 30 min. The reaction mixture is allowed to cool to room temperature and is concentrated in vacuo. The resulting brown oil is purified via column chromatography (CHCl₃/methanol, 95/5, 90/10; CHCl₃/methanol/NH₄OH, 90/10/1) to yield 2.703 g of the title compound as a yellow oil. Physical characteristics. ¹H NMR (400 MHz, DMSO-d₆) δ 7.56-7.55, 7.51, 6.44, 5.07, 4.58-4.55, 2.62-2.56, 2.30; MS (ESI+) m/z 142 (M+H)⁺.

Preparation 21.

1-(1-Benzofuran-2-yl)-2-(methylamino)ethanol.

Potassium hydroxide (9.2 g), and water (3.7 mL) are added to acetonitrile (125 mL). Benzofuran-2-carbaldehyde (12.0 g) is dissolved in the acetonitrile mixture. Trimethylsulfonium iodide (16.7 g) is added, and the mixture is stirred at 60° C. for 3 h. The reaction mixture is cooled to room temperature and filtered. The filtrate is washed with diethyl ether and filtered. This process is repeated until no more KI precipitated. The resulting crude epoxide is concentrated in vacuo and dissolved in a 2.0 M solution of methylamine in methanol (410 mL). The mixture is stirred at room temperature for 18 hours, then concentrated in vacuo to a brown oil. The oil is purified by column chromatography (CHCl₃/methanol, 95/5, 90/10; CHCl₃/methanol/NH₄OH, 89/10/1, 79/20/1) to afford 3.0 g of the title compound as an off-white solid. Physical characteristics. ¹H NMR (300 MHz, DMSO-d₆) δ 7.59, 7.53, 7.24, 6.47, 5.62, 4.77, 2.83, 2.32.

Preparation 22.

2-(Methylamino)-1-thien-2-ylethanol.

Thiophene-2-carboxaldehyde (8.5 g) is dissolved in acetonitrile (115 mL). Trimethylsulfonium iodide (15.5 g), potassium hydroxide (8.5 g), and water (3.4 mL) are added, and the mixture is stirred at 60° C. for 3 h. The reaction mixture is cooled to room temperature and filtered. The retentate is washed with diethyl ether and filtered. This process is repeated until no more KI precipitated. The resulting crude epoxide is concentrated in vacuo and distilled using a Kugelrohr distillation apparatus (0.8 Torr, oven temperature 50° C.). The crude epoxide is dissolved in a 2.0 M solution of methylamine in methanol (152 mL). The mixture is stirred at room temperature for 18 hours, then concentrated in vacuo to a yellow oil. The oil is purified by column chromatography (CHCl₃/methanol, 95/5, 90/10; CHCl₃/methanol/NH₄OH, 89/10/1) to afford 1.8 g of the title compound as a yellow oil that solidifies on standing. Physical characteristics. ¹H NMR (300 MHz, DMSO-d6) δ 7.38, 6.95, 5.62, 4.86, 3.34, 2.67, 2.31.

Preparation 23.

2-(Methylamino)-1-(1H-pyrrol-2-yl)ethanol.

2-chloro-1-(1H-pyrrol-2-yl)ethanol (Croce, P. D.; Ferraccioli, R.; Ritieni, A. Synthesis, 1990, 212-213) (2.04 g) is dissolved in methanol (60 mL) and added dropwise to a 2.0 M solution of methylamine in methanol (71 mL) at 0° C. The reaction mixture is stirred at 0° C. for 1 h and then allowed to warm to room temperature. The reaction mixture is stirred at room temperature for 18 h and then cooled to 0° C. Sodium borohydride (0.806 g) in water (40 mL) is added dropwise. The reaction mixture is stirred at 0° C. for 1 h and then allowed to warm to room temperature. The reaction mixture is stirred at room temperature for 4 h and then concentrated in vacuo to remove methanol. Water (50 mL) is added and the aqueous layer is extracted with ethyl acetate (4×100 mL). The combined organic layers are dried (MgSO₄), filtered, and concentrated in vacuo. The resulting brown oil is purified via column chromatography (CHCl₃/methanol, 95/5, 90/10; CHCl₃/methanol/NH₄OH, 90/10/1) to yield 1.458 g of the title compound as a yellow oil. Physical characteristics. ¹H NMR (400 MHz, DMSO-d₆) δ 10.61, 6.61-6.59, 5.91-5.86, 5.01, 4.60-4.57, 2.73-2.68, 2.64-2.60, 2.30.

Preparation 24.

2-(Methylamino)-1-(1-methyl-1H-pyrrol-2-yl)ethanol.

2-Chloro-1-(1-methyl-1H-pyrrol-2-yl)ethanol (Croce, P. D.; Ferraccioli, R.; Ritieni, A. Synthesis, 1990, 212-213) (2.05 g) is dissolved in methanol (40 mL) and added dropwise to a 2.0 M solution of methylamine in methanol (65 mL) at 0° C. The reaction mixture is stirred at 0° C. for 1 h and then allowed to warm to room temperature. The reaction mixture is stirred at room temperature for 18 h and then cooled to 0° C. Sodium borohydride (0.738 g) in water (40 mL) is added dropwise. The reaction mixture is stirred at 0° C. for 30 min and then allowed to warm to room temperature. The reaction mixture is stirred at room temperature for 18 h. An additional 0.738 g (19.5 mmol) of sodium borohydride is added and the reaction mixture is stirred at room temperature for 18 h. The reaction is quenched with a 1 N HCl solution and then concentrated in vacuo to remove methanol. The aqueous layer is adjusted to pH 12 with a 2 N NaOH solution and extracted with CH₂Cl₂ (4×100 mL). The combined organic layers are dried (MgSO₄), filtered, and concentrated in vacuo. The resulting yellow oil is crystallized from ethyl acetate to yield 0.772 g of the title compound as a white solid. Physical characteristics. M.p. 64-66° C.; ¹H NMR (400 MHz, DMSO-d₆) δ 6.63-6.62, 5.90-5.86, 5.00, 4.62-4.59, 3.59, 2.81-2.68, 2.32; MS (ESI+) m/z 155 (M+H)⁺.

Preparation 25.

2-(Methylamino)-1-(1-methyl-1H-imidazol-4-yl)ethanol.

Potassium hydroxide (5.83 g) and water (0.23 mL) are added to acetonitrile (125 mL). Trimethylsulfonium iodide (10.6 g) and 4(5)-imidazole carboxaldehyde (5.00 g) are then added. The reaction mixture is heated to 60° C. for 3 h. The reaction mixture is allowed to cool to room temperature and filtered. The filtrate is added to a 2.0 M solution of methylamine in methanol (250 mL). The reaction mixture is stirred at room temperature for 18 h. The reaction mixture is concentrated in vacuo. The resulting brown oil is purified via column chromatography (CHCl₃/methanol, 95/5, 90/10; CHCl₃/methanol/NH₄OH, 90/10/1) to yield 0.145 g of the title compound as a yellow oil. Physical characteristics. ¹H NMR (400 MHz, DMSO-d₆) δ 7.44, 6.91, 4.54-4.49, 3.60, 2.72-2.58, 2.30; MS (ESI+) m/z 156 (M+H)⁺.

Preparation 26.

2-(Methylamino)-1-(1-triphenylmethyl-1H-imidazol-4-yl)ethanol.

Sodium hydride (1.18 g, 60% oil dispersion) and DMSO (20 mL) are combined and heated to 70° C. for 1.5 h. The reaction mixture is cooled to room temperature and THF (20 mL) is added. The reaction mixture is cooled to 0° C. and trimethylsulfonium iodide (6.04 g) in DMSO (25 mL) is added. The reaction mixture is allowed to warm to room temperature and 4-(N-Triphenylmethyl)imidazole carboxaldehyde (Bernabe, M.; Burger, A. J. Med. Chem., 1971, 14, 883-885) is added. The reaction mixture is stirred at room temperature for 1 h and then poured into cold water (200 mL)/Et₂O (100 mL). The resulting pale yellow solid is filtered and added to a 2.0 M solution of methylamine in methanol (150 mL). The reaction mixture is stirred at room temperature for 18 h and then concentrated in vacuo. The resulting yellow oil is purified via column chromatography (CHCl₃/methanol, 95/5, 90/10; CHCl₃/methanol/NH₄OH, 90/10/1). The resulting solid is triturated with ethyl acetate to yield 0.288 g of the title compound as a white solid. Physical characteristics. M.p. 169-171° C.; ¹H NMR (400 MHz, DMSO-d₆) δ 7.43-7.38, 7.27, 7.09-7.07, 6.70, 4.98, 4.54-4.51, 2.75-2.71, 2.61-2.56, 2.29; MS (ESI+) m/z 384 (M+H)⁺.

Preparation 27.

1-(3a,7a-Dihydro-1H-indol-3-yl)-2-(methylamino)ethanol.

2-Bromo-1-(1H-indol-3-yl)ethanone (Guella, G.; Mancini, I.; N'Diaye, I.; Pietra, F. Helv. Chim. Acta., 1994, 77, 1999-2006) (3.50 g) is added to a 2.0 M solution of methylamine in methanol (55 mL) at 0° C. The reaction mixture is stirred at 0° C. for 30 minutes and then allowed to warm to room temperature. The reaction mixture is allowed to stir at room temperature for 1 h. The reaction mixture is cooled to 0° C. and sodium borohydride (0.461 g) is added. The reaction mixture is stirred at 0° C. for 30 min and then at room temperature for 18 h. An additional 50 mL of methanol is added and the reaction mixture is heated to 45° C. for 1 h. After 1 h, additional sodium borohydride (0.461 g) is added and heating at 45° C. is continued for 1 h. The reaction mixture is allowed to cool to room temperature and water is added. The mixture is concentrated in vacuo to remove methanol. The aqueous layer is extracted with CH₂Cl₂ (4×100 mL). The combined organic layers are concentrated in vacuo. The resulting orange solid is purified via column chromatography (CHCl₃/methanol, 95/5) to yield the 1.002 g of 1-(1H-indol-3-yl)-2-(methylamino)ethanone as a tan solid. Physical characteristics. ¹H NMR (400 MHz, DMSO-d₆) δ 11.96 (br, 1H), 8.38 (s, 1H), 8.20-8.18 (m, 1H), 7.49-7.46 (m, 1H), 7.23-7.17 (m, 2H), 3.83 (s, 2H), 2.34 (s, 3H); MS (ESI+) m/z 189 (M+H)⁺.

1-(1H-indol-3-yl)-2-(methylamino)ethanone (1.00 g) is dissolved in ethanol (120 mL) and H₂O (30 mL) and cooled in an ice bath. Sodium borohydride (0.402 g) is added and the reaction mixture is allowed to warm to room temperature. The reaction mixture is stirred at room temperature for 4 h. The reaction mixture is then cooled in an ice bath and an additional 0.402 g of sodium borohydride is added. The reaction mixture is allowed to warm to room temperature and is stirred for 18 h. The reaction mixture is cooled in an ice bath and acetone (7 mL) is added. The reaction mixture is concentrated in vacuo and H₂O (50 mL) is added to the residue. The aqueous layer is adjusted to pH 12 with a 2 N NaOH solution and then extracted with CH₂Cl₂ (4×100 mL). The combined organic layers are dried (MgSO₄), filtered, and concentrated in vacuo. The resulting pale yellow solid is triturated with ethyl acetate to yield 0.415 g of the title compound as a pale yellow solid. Physical characteristics. M.p. 117-120° C.; ¹HNMR (400 MHz, DMSO-d6) δ 10.88, 7.63-7.61, 7.35-7.33, 7.21-7.20, 7.07-7.03, 6.97-6.94, 4.93-4.90, 2.86-2.72, 2.36; MS (ESI+) m/z 191 (M+H)⁺.

Preparation 28.

2-Bromo-1-(2,5-dimethyl-3-furyl)ethanone.

3-Acetyl-2,5-dimethylfuran (13.3 mL) is dissolved in 1/2 dioxane/Et₂O (600 mL) and cooled to 0° C. Bromine (16.0 g) is added dropwise over 1 h. The reaction mixture is stirred at 0° C. for 1 h and then allowed to warm to room temperature. The reaction mixture is stirred at room temperature for 18 h. The reaction mixture is cooled to 0° C. and an additional 1.0 mL of bromine is added. The reaction mixture is allowed to warm to room temperature and is stirred for 2 h. The reaction is quenced with a saturated ammonium chloride solution (100 mL). The organic layer is removed and the aqueous layer extracted with Et₂O (2×100 mL). The combined organic layers are dried (MgSO₄), filtered, and concentrated in vacuo. The resulting brown, oily solid is purified via column chromatography (hexanes/CH₂Cl₂; 70/30) to yield 14.23 g of an impure yellow oily solid which is recrystallized from hexanes to yield 7.52 g of the title compound as a white solid. Physical characteristics. M.p. 56-58° C.; ¹H NMR (400 MHz, DMSO-d₆) δ 6.49, 4.54, 2.50, 2.08; ¹³C NMR (100 MHz, CDCl₃) δ 187.2, 159.3, 150.5, 118.9, 105.6, 33.2, 14.4, 13.2;

Preparation 29.

1-(2,5-Dimethyl-3-furyl)-2-(methylamino)ethanol.

2-Bromo-1-(2,5-dimethyl-3-furyl)ethanone (7.30 g) is dissolved in methanol (80 mL) and added dropwise to a 2.0 M solution of methylamine in methanol (168 mL) at 0° C. The reaction mixture is stirred at 0° C. for 30 min and then sodium borohydride (1.91 g) in water (40 mL) is added dropwise. The reaction mixture is stirred at 0° C. for 1.5 h and then allowed to warm to room temperature. The reaction mixture is stirred at room temperature for 18 h. An additional 0.636 g of sodium borohydride is added and stirring is continued for 3 h. The reaction is quenched with a 1 N HCl solution and concentrated in vacuo to remove methanol. The residue is poured into cold 2 N NaOH (100 mL)/ethyl acetate (200 mL). The organic layer is removed and the aqueous layer extracted with ethyl acetate (3×200 mL). The combined organic layers are dried (MgSO₄), filtered, and concentrated in vacuo. The resulting yellow oil is purified via column chromatography (CHCl₃/methanol, 95/5, 90/10; CHCl₃/methanol/NH₄OH, 90/10/1). The resulting pale yellow solid is recrystallized from ethyl acetate to yield 2.406 g of the title compound as a white solid. Physical characteristics. M.p. 76-77° C.; ¹H NMR (400 MHz, DMSO-d₆) δ 5.93, 4.82, 4.47-4.43, 2.64-2.54, 2.46-2.42, 2.32, 2.16; MS (ESI+) m/z 170 (M+H)⁺.

Preparation 30.

1-(3a,7a-Dihydro-1-benzothien-3-yl)-2-(methylamino)ethanol.

Potassium hydroxide (3.11 g) and water (0.12 mL) are added to acetonitrile (50 mL). Trimethylsulfonium iodide (5.65 g) and thianapthene-3-carboxaldehyde (4.50 g) are then added. The reaction mixture is heated to 60° C. for 4 h. The reaction mixture is allowed to cool to room temperature and is then diluted with Et₂O (25 mL). The precipitate is filtered off, and the filtrate is concentrated in vacuo. The resulting crude epoxide (6.20 g) is dissolved in methanol (40 mL) and added to a 2.0 M solution of methyl amine in methanol (100 mL). The reaction mixture is stirred at room temperature for 3 d. The reaction mixture is concentrated in vacuo. The resulting brown oil is purified via column chromatography (CHCl₃/methanol, 95/5, 90/10; CHCl₃/methanol/NH₄OH, 90/10/1) to yield 1.753 g of the title compound as a yellow solid. Physical characteristics. M.p. 98-102° C.; ¹H NMR (400 MHz, DMSO-d₆) δ 7.98-7.90, 7.51, 7.60, 7.40-7.33, 5.43, 5.04, 2.80, 2.34; MS (ESI+) m/z 208 (M+H)⁺.

Preparation 31.

2-(Methylamino)-1-(1-methyl-1H-indol-2-yl)ethanol.

Potassium hydroxide (3.18 g) and water (0.13 mL) are added to acetonitrile (50 mL). Trimethylsulfonium iodide (5.78 g) and 1-methylindole-2-carboxaldehyde (4.50 g) are then added. The reaction mixture is heated to 60° C. for 3 h. The reaction mixture is allowed to cool to room temperature and is diluted with Et₂O (25 mL). The precipitate is filtered off, and the filtrate is concentrated in vacuo. The resulting crude epoxide (5.50 g) is dissolved in methanol (30 mL) and added to a 2.0 M solution of methylamine in methanol (100 mL). The reaction mixture is stirred at room temperature for 3 d and then heated to reflux for 2 h. The reaction mixture is allowed to cool to room temperature and is concentrated in vacuo. The resulting brown oil is purified via column chromatography (CHCl₃/methanol, 95/5, 90/10; CHCl₃/methanol/NH₄OH, 90/10/1) to yield 0.100 g of the impure title compound as a yellow solid. Physical characteristics. ¹H NMR (400 MHz, DMSO-d₆) δ 7.49, 7.41, 7.12-7.09, 7.00-6.97, 6.35, 5.36, 4.89-4.85, 3.76, 2.93-2.84, 2.36; MS (ESI+) m/z 205 (M+H)⁺.

Preparation 32.

5-(2-Bromo-1-hydroxyethyl)thiophene-2-carbonitrile.

To a solution of 2-acetyl-5-cyanothiophene (1.5 g) in 20 mL of p-dioxane/ethyl ether, 1:2, v/v) is introduced bromine (0.5 mL). After 2 h, ice water (30 mL) is added. The solid precipitates are collected by filtration and washed with water to afford 1.4 g of a white solid. The filtrate is allowed to stand overnight to give a second crop of 0.86 g of white product for a total yield of 2.26 g 5-(bromoacetyl)thiophene-2-carbonitrile. Physical characteristics. ¹H NMR (DMSO-d₆) δ 8.16, 8.11, 4.94; MS (ESI−) m/z 230 (M−H)⁻.

NaBH₄ (0.46 g in 5 mL of water) is added to a suspension of 5-(bromoacetyl)-thiophene-2-carbonitrile (1.85 g) in methanol (50 mL) cooled to −10° C. After 10 min, 48% aq. HBr is added to adjust the pH to 3. The reaction mixture is concentrated to approximately 25 mL before water (30 mL) is added. The mixture is extracted with dichloromethane (3×40 mL). The organic phases are combined, washed with brine, and dried over MgSO₄. Removal of the solvent gave 1.6 g of the title compound as an orange oil. Physical characteristics. ¹H NMR (DMSO-d₆) δ 7.86, 7.23, 6.67, 5.17, 3.81, 3.68; MS (ESI−) m/z 232 (M−H)⁻.

Preparation 33.

5-(1-Hydroxy-2-(methylamino)ethyl)thiophene-2-carbonitrile.

A solution of methylamine (2.0 M in methanol, 80 mL) is added to a solution of 5-(2-bromo-1-hydroxyethyl)thiophene-2-carbonitrile (Preparation 32, 1.6 g) in methanol (20 mL). The reaction mixture is stirred at room temperature overnight and then is concentrated to nearly dryness. The residue is dissolved in of methanol (20 mL) and is treated with resin (2 g, BioRad AG® 50w-x2, hydrogen form, strongly acidic cation) for 4 hours. The resin is collected by filtration and washed with a large amount of methanol. The resin is washed with 10% N OH/MeOH (100 mL) and the solution is concentrated. The crude product is purified by flash chromatography (silica gel, 1% N140H/110% MeOH/89% CH₂Cl₂) to yield 0.80 g of the title compound as a white solid. Physical characteristics. ¹H NMR (DMSO-d₆) δ 7.81, 7.13, 6.13, 4.93, 2.72, 2.33; MS (ESI+) m/z 183 (M+H)⁺; HRMS (FAB) m/z 183.0600 (M+H)⁺.

Preparation 34.

2-Chloro-1-(1,3-thiazol-2-yl)ethanone.

2-(Trimethylsilyl)thiazole (4.83 g) is dissolved in CH₂Cl₂ (40 mL) and cooled to 0° C. Chloroacetyl chloride (5.1 mL) is added dropwise via syringe with vigorous stirring. After 4 h, sat. aq. NaHCO₃ solution is added until the solution is at neutral pH and the resulting mixture is extracted with CH₂Cl₂. The combined organics are dried (MgSO₄), filtered, and concentrated to afford 4.27 g of the title compound as pale yellow solid. Physical characteristics. ¹H NMR (400 MHz, CDCl₃) δ 7.90-7.88, 7.65-7.64, 4.83.

Preparation 35.

2-(Methylamino)-1-(1,3-thiazol-2-yl)ethanol.

2-Chloro-1-(1,3-thiazol-2-yl)ethanone (Preparation 34, 0.6 g) is dissolved in methanol (4 mL) and cooled to 0° C. Sodium borohydride (0.3 g) in methanol (4 mL) is stirred 1 h and is then added to the ketone dropwise. The reaction mixture is stirred at 0° C. for 30 min. and then for 1.5 h at room temperature. HCl (1N) is added until pH 4 and then sat. aq. NaHCO₃ is added until neutral pH. The resulting mixture is extracted with CH₂Cl₂. The combined organic layers are dried (MgSO₄), filtered, and concentrated to provide a colorless oil. The resulting oil, methylamine (2.0 M in methanol, 30.0 mL) and NaI (45 mg) are placed in a sealed tube and heated at 60° C. for 16 h. The solution is concentrated and purified by chromatotron (2 mm silica, 99/1 to 90/10 CH₂Cl₂/MeOH) to provide 0.223 g of the title compound as an off-white solid. Physical characteristics. ¹H NMR (400 MHz, DMSO-d₆) δ 7.81-7.80, 7.74-7.73, 6.95, 5.15-5.11, 3.33, 3.14-3.09, 2.58; MS (ESI+) m/z 159 (M+H)⁺.

Preparation 36.

1-(1,3-Benzothiazol-2-yl)-2-bromoethanone.

2-(Trimethylsilyl)benzothiazole (2.0 g, prepared as described in Bull. Chem. Soc. Jpn. 1988, 61, 3637-3648) is dissolved in CH₂Cl₂ (31 mL). Bromoacetylbromide (1.7 mL) in CH₂Cl₂ (17 mL) is added dropwise and stirred for 3 h. The mixture is then neutralized with sat. aq. NaHCO₃ solution and extracted with CH₂Cl₂. The combined organic layers are washed with brine, dried (MgSO₄), filtered and concentrated to afford the title compound. Physical characteristics. ¹H NMR (400 MHz, CDCl₃) δ 8.23-8.21, 8.03-8.01, 7.64-7.57, 4.86.

Preparation 37.

1-(1,3-Benzothiazol-2-yl)-2-(methylamino)ethanol.

1-(1,3-Benzothiazol-2-yl)-2-bromoethanone (Preparation 36, 2.0 g) is dissolved in methanol (10 mL) and cooled to 0° C. Sodium borohydride (0.55 g) is added to methanol (10 mL), stirred for 30 min, and is then added dropwise to the ketone solution. After 30 min, the reaction is quenched with 1N HCl, neutralized with sat. aq. NaHCO₃ solution and extracted with CH₂Cl₂. The combined organic layers are dried (MgSO₄), filtered and concentrated. The residue is dissolved in methanol (5 mL), cooled to 0° C. and methylamine (2.0 M in methanol, 49 mL) is added dropwise. The mixture is stirred at room temperature for 2 h and is then concentrated. The resulting oil is purified by column chromtography (CH₂Cl₂ to 90/10 CH₂Cl₂/MeOH) to yield 0.289 g of the title compound as a pasty solid. Physical characteristics: ¹H NMR (400 MHz, DMSO-d₆) δ 8.16-8.14, 8.00-7.98, 7.56-7.54, 7.49-7.45, 5.35-5.31, 3.52-3.49, 3.33-3.27, 2.54; MS (ESI+) m/z 209 (M+H)⁺.

Preparation 38.

(Methyl(trityl)amino)acetaldehyde.

Triethylamine (9.3 mL) is added to a mixture of 2-methylaminoethanol (5.0 g) and triphenylmethyl chloride (18.6 g) in CH₂Cl₂ (150 mL) and the solution is stirred at room temperature for 2 h. The solvent is removed in vacuum and the residue is dissolved in EtOAc (200 mL). The solution is washed with water (100 mL) and brine (50 mL), dried (MgSO₄), filtered and concentrated. The residue is chromatographed on silica gel, eluting with 20% EtOAc in n-heptane, to afford 18.7 g of 2-[methyl(trityl)amino]ethanol as a white solid. Physical characteristics. ¹H NMR (400 MHz, DMSO-d₆) δ 7.45-7.14, 4.56, 3.65, 2.12, 1.99.

A solution of DMSO (1.47 mL) in CH₂Cl₂ (60 mL) is cooled to −78° C. and oxalyl chloride is added dropwise over 10 min. A solution of 2-tritylmethylaminoethanol (3.0 g) in CH₂Cl₂ (10 mL) is added dropwise and the resulting mixture is stirred for 15 min. Triethylamine (6.6 mL) is added and the mixture is allowed to warm to room temperature. The reaction mixture is quenched by adding water and is then diluted with CH₂Cl₂ (100 mL). The organic phase is washed with water and brine, dried (Na₂SO₄), filtered and concentrated. The residue is triturated with ether to afford a quantitative yield of the title compound as white solid. Physical characteristics. ¹H NMR (400 MHz, DMSO-d₆) δ 7.44-7.15, 4.55, 3.66-3.62, 2.11, 1.99.

Preparation 39.

2-(Methylamino)-1-(1H-pyrazol-5-yl)ethanol Dihydrochloride.

Sodium hydride (5.38 g, 60% dispersion in mineral oil) is added to a cooled (5° C.) solution of pyrazole (6.10 g) in anhydrous DMF (50 mL) over a 30 min period. The resulted suspension is stirred for an additional 30 min and 2-(trimethylsilyl)ethoxy-methyl chloride (19 mL) is added dropwise over 10 min. The resulted mixture is stirred for 3 h at room temperature. The reaction mixture is quenched by adding water and is then extracted with EtOAc (200 mL). The organic layer is washed with water (3×100 mL) and brine (100 mL), dried (MgSO₄), filtered and concentrated to afford 1-((2-(trimethylsilyl)ethoxy)methyl)-1H-pyrazole as clear oil. Physical characteristics. ¹H NMR (400 MHz, CDCl₃) δ 7.63-7.55, 6.32, 5.43, 3.54, 0.88, −0.05.

n-BuLi (2.5 M solution, 1.8 mL) is added dropwise to a cold (−78° C.) solution of 1-((2-(trimethylsilyl)ethoxy)methyl)-1H-pyrazole (0.80 g) in THF/ether (50 mL, 3/2). After 30 min, a solution of (methyl(trityl)amino)acetaldehyde (Preparation 38, 1.27 g) in THF/ether (5 mL, 3/2) is added. The reaction mixture is stirred for 1 h at −78° C. and then allowed to warmed to room temperature. The reaction is quenched by adding sat. aq. NH₄Cl and water dropwise. The resulting suspension is extracted with EtOAc (2×100 mL). The combined organic layers are washed with water (100 mL) and brine (50 mL), dried (Na₂SO₄), filtered and concentrated. The residue is purified by chromatography on silica gel, eluting with 20% EtOAc in n-heptane, to afford 0.7 g of 2-(methyl(trityl)amino)-1-(1-((2-(trimethylsilyl)ethoxy)methyl)-1H-pyrazol-5-yl)ethanol as a white solid. Physical characteristics. ¹H NMR (400 MHz, DMSO-d₆) δ 7.58-7.49, 7.45-7.43, 7.36-7.32, 7.24-7.21, 6.21, 5.76, 5.56-5.46, 5.22-5.20, 3.50-3.45, 2.90-2.80, 2.15, 2.09-1.98, 0.74-0.70, 0.00.

A solution of 4 N HCl in dioxane (20 mL) is added to a solution of 2-(methyl(trityl)-amino)-1-(1-((2-(trimethylsilyl)ethoxy)methyl)-1H-pyrazol-5-yl)ethanol (0.7 g) in MeOH (20 mL) and the mixture is refluxed overnight. After cooling to room temperature, the solvent is removed by evaporation in vacuum and the residue is suspended in EtOAc. The resulting solids are collected by filtration, washed with hot EtOAc, and dried under vacuum to afford 0.23 g of the title compound as a white solid. Physical characteristics. ¹H NMR (400 MHz, DMSO-d₆) δ 9.26, 8.83, 7.77, 6.36, 5.04, 3.25-3.09, 2.58.

### Example 1

rac-N-(4-Chlorobenzyl)-2-(((2-(2-furyl)-2-hydroxyethyl)(methyl)amino)methyl)-7-methyl-4-oxo-4,7-dihydrothieno[2,3-b]pyridine-5-carboxamide.

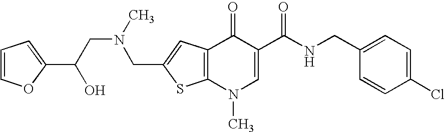

N-(4-Chlorobenzyl)-2-(chloromethyl)-7-methyl-4-oxo-4,7-dihydrothieno[2,3-b]pyridine-5-carboxamide (Preparation 1, 0.250 g) is suspended in DMF (14 mL), and N,N-diisopropylethylamine (0.23 mL) and 1-(2-furyl)-2-(methylamino)ethanol (Preparation 17, 0.231 g) are added. The reaction mixture is heated to 90° C. for 2 h. The reaction mixture is allowed to cool to room temperature and is poured into water (50 mL). The resulting off-white solid is filtered and purified by column chromatography (CH₂Cl₂/methanol, 99/1) to yield 0.173 g of the title compound as a white solid. Physical characteristics. M.p. 161-166° C.; ¹H NMR (400 MHz, DMSO-d₆) δ 10.61, 8.70, 7.41-7.31, 6.40-6.39, 6.29-6.28, 5.30, 4.75, 4.55, 3.93, 3.83, 2.81-2.71, 2.27; ¹³C NMR (100 MHz, CDCl₃) δ 173.0, 164.9, 153.8, 150.6, 144.7, 142.4, 137.3, 132.8, 131.5, 128.9, 128.7, 122.0, 115.8, 110.3, 107.2, 63.8, 60.9, 57.2, 43.2, 42.6, 41.9; MS (ESI+) m/z 486 (M+H)⁺. Anal. Found: C, 59.00; H, 5.04; N, 8.58; Cl, 7.28; S, 6.57.

### Example 2

(+)—N-(4-Chlorobenzyl)-2-((((R)-2-(2-furyl)-2-hydroxyethyl)(methyl)amino)-methyl)-7-methyl-4-oxo-4,7-dihydrothieno [2,3-b]pyridine-5-carboxamide.

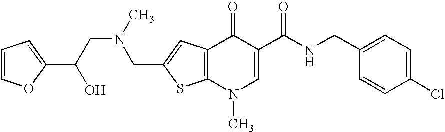

rac-N-(4-Chlorobenzyl)-2-(((2-(2-f l)-2-hydroxyethyl)(methyl)amino)methyl)-7-methyl-4-oxo-4,7-dihydrothieno[2,3-b]pyridine-5-carboxamide (Example 1) is resolved preparatively on a 5×50 cm Chiralcel OD-H column (Chiral Technologies), at a column temperature of 30° C. The mobile phase is 50% isopropyl alcohol/50% heptane (v/v) with a flow rate of 70 mL/min. Peaks are detected by UV at 250 nm. A 487 mg sample is injected in 10 mL of 50% IPA/50% CHCl₃ (v/v). The more slowly eluting enantiomer is isolated and then further purified by column chromatography (CH₂Cl₂/methanol, 99/1). The resulting pale yellow solid is recrystallized from methanol to yield 0.124 g of the title compound as a pale yellow solid. Physical characteristics. M.p. 159-162° C.; ¹H NMR (400 MHz, DMSO-d₆) δ 10.61, 8.70, 7.56, 7.41-7.31, 6.40-6.39, 6.30-6.29, 5.30, 4.76-4.72, 4.55, 3.93, 3.83, 2.81-2.72, 2.27; MS (ESI+) m/z 486 (M+H)⁺; HRMS (FAB) m/z 486.1266 (M+H)⁺; [α]²⁵_{D}=+16 (c 0.74, methylene chloride). Anal. Found: C, 57.74; H, 5.25; N, 8.47; Cl, 7.03; S, 6.36.

### Example 3

rac-N-(4-Chlorobenzyl)-2-(((2-hydroxy-2-(5-methyl-2-furyl)ethyl)(methyl)amino)methyl)-7-methyl-4-oxo-4,7-dihydrothieno[2,3-b]pyridine-5-carboxamide.

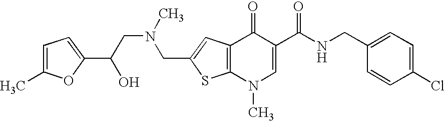

N-(4-Chlorobenzyl)-2-(chloromethyl)-7-methyl-4-oxo-4,7-dihydrothieno[2,3-b]pyridine-5-carboxamide (Preparation 1, 0.500 g) is suspended in DMF (30 mL), and N,N-diisopropylethylamine (0.46 mL) and 2-(methylamino)-1-(5-methyl-2-furyl)ethanol (Preparation 19, 0.407 g) are added. The reaction mixture is heated to 90° C. for 2 h. The reaction mixture is allowed to cool to room temperature and is poured into water (100 mL). The resulting pale yellow solid is filtered and purified by column chromatography (CH₂Cl₂/methanol, 99/1) to yield 0.561 g of a pale yellow solid which is recrystallized from acetonitrile to yield 0.527 g of the title compound as a white solid. Physical characteristics. M.p. 137-140° C.; ¹H NMR (400 MHz, DMSO-d₆) δ 10.61, 8.70, 7.41-7.31, 6.14-6.13, 5.99-5.98, 5.20, 4.68-4.63, 4.55, 3.93, 3.86, 2.79-2.68, 2.27, 2.17; ¹³C NMR (100 MHz, CDCl₃) δ 173.0, 165.0, 152.2, 151.9, 150.6, 148.4, 144.7, 138.1, 137.3, 132.7, 131.5, 128.9, 128.6, 121.7, 115.7, 108.2, 106.1, 63.8, 61.0, 57.2, 43.1, 42.5, 41.8, 13.6; MS (ESI+) m/z 500 (M+H)⁺; HRMS (FAB) m/z 500.1422 (M+H)⁺. Anal. Found: C, 59.28; H, 5.44; N, 8.69; Cl, 6.89; S, 6.21.

### Example 4

rac-N-(4-Chlorobenzyl)-2-(((2-(3-furyl)-2-hydroxyethyl)(methyl)amino)methyl)-7-methyl-4-oxo-4,7-dihydrothieno[2,3-b]pyridine-5-carboxamide. 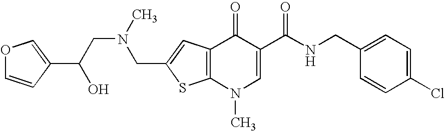
1-(3-Furyl)-2-(methylamino)ethanol (Preparation 20, 0.370 g) is dissolved in DMF (30 mL), and N,N-diisopropylethylamine (0.46 mL) and N-(4-chlorobenzyl)-2-(chloro-methyl)-7-methyl-4-oxo-4,7-dihydrothieno[2,3-b]pyridine-5-carboxamide (Preparation 1, 0.500 g) are added. The reaction mixture is heated to 90° C. for 2 h. The reaction mixture is allowed to cool to room temperature and is poured into water (1.00 mL). The resulting off-white solid is filtered and purified by column chromatography (CH₂Cl₂/methanol; 99/1, 98/2) to yield 0.457 g of a pale yellow solid which is recrystallized from ethyl acetate to yield 0.400 g of the title compound as a pale yellow solid. Physical characteristics. M.p. 151-153° C.; ¹H NMR (400 MHz, DMSO-d₆) δ 10.61, 8.70, 7.58-7.55, 7.41-7.32, 6.45, 5.03, 4.74-4.70, 4.55, 3.94, 3.86, 2.70-2.59, 2.29; ¹³C NMR (100 MHz, CDCl₃) δ 173.0, 164.9, 150.5, 144.7, 143.4, 139.4, 137.9, 137.3, 132.8, 131.5, 128.9, 128.6, 125.9, 121.7, 115.8, 108.4, 63.6, 62.9, 57.3, 43.2, 42.6, 41.9; MS (ESI+) m/z 486 (M+H)⁺; HRMS (FAB) m/z 486.1257 (M+H)⁺. Anal. Found: C, 59.23; H, 4.99; N, 8.59; Cl, 7.19; S, 6.46.

### Example 5

rac-2-(((2-(1-Benzofuran-2-yl)-2-hydroxyethyl)(methyl)amino)methyl)-N-(4-chlorobenzyl)-7-methyl-4-oxo-4,7-dihydrothieno[2,3-b]pyridine-5-carboxamide.

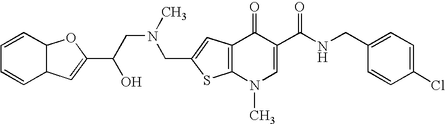

N-(4-Chlorobenzyl)-2-(chloromethyl)-7-methyl-4-oxo-4,7-dihydrothieno[2,3-b]pyridine-5-carboxamide (Preparation 1, 0.5 g), 1-(1-benzofuran-2-yl)-2-(methylamino)ethanol (Preparation 21, 0.5 g) and N,N-diisopropylethylamine (0.91 mL) are added to dimethylformamide (28 mL The mixture is stirred at 90° C. for 2 h. The reaction mixture is cooled to room temperature and poured into water (100 mL). An off-white precipitate forms and is filtered. The precipitate is adsorbed on silica and purified by column chromatography (MeOH/CH₂Cl₂, 1%) to afford a white solid. The crude material is recrystallized from methanol to afford 0.36 g of the desired product as a white solid. Physical characteristics: M.p. 212-214° C.; ¹H NMR (300 MHz, DMSO-d6) δ 10.62, 8.65, 7.59, 7.41, 7.38, 7.35, 6.77, 5.60, 4.53, 3.81, 3.68, 2.94, 2.76, 2.33; HRMS (FAB) m/z 536.1406 (M+H)⁺. Anal. Found: C, 62.57; H, 4.88; N, 7.80; Cl, 6.62; S, 5.95.

### Example 6

rac-N-(4-Chlorobenzyl)-2-(((2-hydroxy-2-thien-2-ylethyl)(methyl)amino)methyl)-7-methyl-4-oxo-4,7-dihydrothieno[2,3-b]pyridine-5-carboxamide.

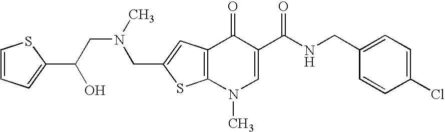

N-(4-Chlorobenzyl)-2-(chloromethyl)-7-methyl-4-oxo-4,7-dihydrothieno[2,3-b]pyridine-5-carboxamide (Preparation 1, 0.5 g), 2-(methylamino)-1-thien-2-ylethanol (Preparation 22, 0.41 g) and N,N-diisopropylethylamine (0.91 mL) are added to dimethylformamide (28 mL). The mixture is stirred at 90° C. for 2 h. The reaction mixture is cooled to room temperature and poured into water (100 mL). An off-white precipitate forms and is filtered. The precipitate is adsorbed on silica and purified by column chromatography (MeOH/CH₂Cl₂, 3%) to afford 0.51 g of the title compound as a yellow foam. Physical characteristics: ¹H NMR (300 MHz, DMSO-d6) δ 10.60, 8.70, 7.39, 7.35, 7.32, 6.97, 5.60, 4.54, 3.93, 3.87, 2.70, 2.31; MS (ESI+) m/z 502 (M+H)⁺. Anal. Found: C, 57.20; H, 4.97; N, 8.30; Cl, 7.01; S, 12.54.

### Example 7

rac-N-(4-Chlorobenzyl)-2-(((2-hydroxy-2-(1H-pyrrol-2-yl)ethyl)(methyl)amino)-methyl)-7-methyl-4-oxo-4,7-dihydrothieno[2,3-b]pyridine-5-carboxamide.

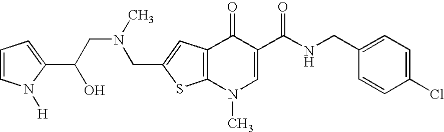

2-(Methylamino)-1-(1H-pyrrol-2-yl)ethanol (Preparation 23, 0.541 g) is dissolved in DMF (30 mL), and N,N-diisopropylethylamine (0.46 mL) and N-(4-chlorobenzyl)-2-(chloromethyl)-7-methyl-4-oxo-4,7-dihydrothieno[2,3-b]pyridine-5-carboxamide (Preparation 1, 0.500 g) are added. The reaction mixture is heated to 90° C. for 2 h. The reaction mixture is allowed to cool to room temperature and is poured into water (100 mL). The resulting tan solid is filtered and purified by column chromatography (CH₂Cl₂/methanol; 99/1, 98/2). The resulting off-white solid is recrystallized from acetonitrile to yield 0.297 g of the title compound as an off-white solid. Physical characteristics. M.p. 169-172° C. (dec); ¹H NMR (400 MHz, DMSO-d₆) δ 10.63-10.60, 8.70, 7.41-7.31, 6.63-6.61, 5.92-5.90, 4.94, 4.76-4.71, 4.55, 3.97, 3.84, 2.78-2.64, 2.08; ¹³C NMR (100 MHz, CDCl₃) δ 172.9, 164.9, 150.4, 144.6, 137.8, 137.3, 132.8, 131.6, 131.4, 128.9, 128.7, 121.8, 117.4, 115.8, 108.4, 104.6, 64.2, 63.1, 58.7, 57.3, 43.1, 42.6, 42.1; MS (ESI+) m/z 485 (M+H)⁺; HRMS (FAB) m/z 485.1427 (M+H)⁺. Anal. Found: C, 59.20; H, 5.26; N, 11.48; Cl, 7.21; S, 6.48.

### Example 8

rac-N-(4-Chlorobenzyl)-2-(((2-hydroxy-2-(1-methyl-1H-pyrrol-2-yl)ethyl)-(methyl)amino)methyl)-7-methyl-4-oxo-4,7-dihydrothieno[2,3-b]pyridine-5-carboxamide.

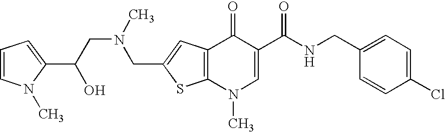

N-(4-Chlorobenzyl)-2-(chloromethyl)-7-methyl-4-oxo-4,7-dihydrothieno[2,3-b]pyridine-5-carboxamide (Preparation 1, 0.500 g) is suspended in DMF (30 mL), and N,N-diisopropylethylamine (0.46 mL) and 2-(methylamino)-1-(1-methyl-1H-pyrrol-2-yl)ethanol (Preparation 24, 0.404 g) are added. The reaction mixture is heated to 90° C. for 1 h. The reaction mixture is allowed to cool to room temperature and is poured into water (100 mL). The resulting tan solid is filtered and purified by column chromatography (CH₂Cl₂/methanol, 99/1) to yield 0.443 g of a pale yellow solid which is recrystallized from methanol then ethyl acetate to yield 0.322 g of the title compound as an off-white solid. Physical characteristics. M.p. 140-145° C.; ¹H NMR (400 MHz, DMSO-d₆) δ 10.61, 8.70, 7.41-7.33, 6.63-6.62, 5.91-5.90, 4.55, 3.94, 3.90-3.82, 3.58, 2.83-2.73, 2.30; ¹³C NMR (100 MHz, CDCl₃) δ 173.0, 164.9, 150.5, 148.4, 144.7, 138.1, 137.3, 132.8, 131.5, 131.4, 128.9, 128.6, 123.4, 121.7, 115.8, 106.8, 106.0, 62.0, 61.2, 57.4, 43.1, 42.5, 42.0, 34.1; MS (ESI+) m/z 499 (M+H)⁺; HRMS (FAB) m/z 499.1584 (M+H)⁺. Anal. Found: C, 59.94; H, 5.56; N, 10.94; Cl, 6.94; S, 6.25.

### Example 9

rac-N-(4-Chlorobenzyl)-2-(((2-hydroxy-2-(1-methyl-1H-imidazol-4-yl)ethyl) (methyl)amino)methyl)-7-methyl-4-oxo-4,7-dihydrothieno[2,3-b]pyridine-5-carboxamide.

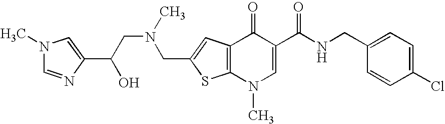

2-(Methylamino)-1-(1-methyl-1H-imidazol-4-yl)ethanol (Preparation 25, 0.145 g) is dissolved in DMF (12 mL), and N,N-diisopropylethylamine (0.18 mL) and N-(4-chlorobenzyl)-2-(chloromethyl)-7-methyl-4-oxo-4,7-dihydrothieno[2,3-b]pyridine-5-carboxamide (Preparation 1, 0.200 g) are added. The reaction mixture is heated to 90° C. for 1 h. The reaction mixture is allowed to cool to room temperature and is poured into water (40 mL) and stored in the freezer for 1 h. The resulting off-white solid is filtered and purified by column chromatography (CH₂Cl₂/methanol, 98/2) to yield 0.091 g of a yellow, gummy solid which is recrystallized from ethyl acetate then acetonitrile to yield 0.044 g of the title compound as a pale yellow solid. Physical characteristics. M.p. 124-130° C.; ¹H NMR (400 MHz, DMSO-d₆) δ 10.62, 8.70, 7.45, 7.41-7.31, 6.93, 4.79, 4.68-4.64, 4.55, 3.95, 3.86, 3.60, 2.77-2.64, 2.30; MS (ESI+) m/z 500 (M+H)⁺, Anal. Found: C, 56.05; H, 5.40; N, 13.59; Cl, 6.92; S, 6.22.

Preparation 40.

rac-N-(4-Chlorobenzyl)-2-(((2-hydroxy-2-(1-triphenymethyl-1H-imidazol-4-yl)ethyl)(methyl)amino)methyl)-7-methyl-4-oxo-4,7-dihydrothieno[2,3-b]pyridine-5-carboxamide.

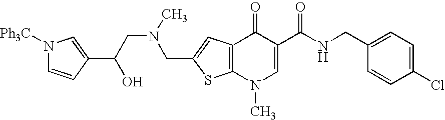

N-(4-Chlorobenzyl)-2-(chloromethyl)-7-methyl-4-oxo-4,7-dihydrothieno[2,3-b]pyridine-5-carboxamide (Preparation 1, 0.200 g) is suspended in DMF (11 mL), and N,N-diisopropylethylamine (0.11 mL) and 2-(methylamino)-1-(1-triphenylmethyl-1H-imidazol-4-yl)ethanol (Preparation 26, 0.242 g) are added. The reaction mixture is heated to 90° C. for 2 h. The reaction mixture is allowed to cool to room temperature and is poured into water (30 mL). The resulting off-white solid is filtered and purified by column chromatography (CH₂Cl₂/methanol; 98/2, 96/4) to yield 0.268 g of the title compound as a pale yellow solid. Physical characteristics. M.p. 105-112° C.; ¹H NMR (400 MHz, DMSO-d₆) δ 10.62, 8.69, 7.41-7.28, 7.08-7.06, 6.76, 4.90, 4.69-4.65, 4.55, 3.87, 3.82-3.78, 2.84-2.80, 2.69-2.64, 2.27; ¹³C NMR (100 MHz, CDCl₃) δ 173.0, 165.0, 150.5, 144.5, 142.3, 138.6, 137.3, 132.7, 131.6, 129.8, 128.9, 128.6, 128.0, 121.4, 118.5, 115.7, 65.1, 62.8, 57.3, 43.1, 42.5, 42.0; MS (ESI+) m/z 728 (M+H)⁺; Anal. Found: C, 68.61; H, 5.34; N, 9.45.

### Example 10

rac-N-(4-Chlorobenzyl)-2-(((2-hydroxy-2-(1H-imidazol-4-yl)ethyl)(methyl)amino)methyl)-7-methyl-4-oxo-4,7-dihydrothieno[2,3-b]pyridine-5-carboxamide.

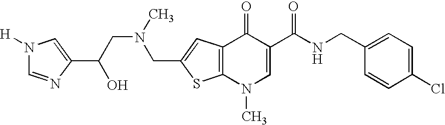

N-(4-Chlorobenzyl)-2-(((2-hydroxy-2-(1-triphenymethyl-1H-imidazol-4-yl)ethyl)-(methyl)amino)methyl)-7-methyl-4-oxo-4,7-dihydrothieno[2,3-b]pyridine-5-carboxamide (Preparation 40, 0.243 g) is dissolved in THF (10 mL) and a 1.0 M solution of HCl in Et₂O (0.37 mL) is added dropwise. The reaction mixture is stirred at room temperature for 20 min. Methanol (2 mL) is added followed by a 1.0 M solution of HCl in Et₂O (0.37 mL). Stirring is continued at room temperature for 1 h, and then methanolic HCl (1 mL) is added. The reaction mixture is stirred at room temperature for 1 h and then heated to 50° C. for 6 h. The reaction mixture is allowed to cool to room temperature. The pale yellow solid that precipitates is filtered and dissolved in water, and the solution is adjusted to pH 8 with a 2 N NaOH solution. The resulting yellow solid is filtered and recrystallized from acetonitrile to yield 0.101 g of the title compound as a pale yellow solid. Physical characteristics. M.p. 164-169 C; ¹H NMR (400 MHz, DMSO-d₆) δ 11.87, 10.62, 8.70, 7.52, 7.41-7.31, 6.88, 4.89, 4.75, 4.55, 3.94, 3.85, 2.80-2.68, 2.29; MS (ESI+) m/z 486 (M+H)⁺. Anal. Found: C, 56.46; H, 5.02; N, 14.24; Cl, 7.36; S, 6.55.

### Example 11

rac-N-(4-Chlorobenzyl)-2-(((2-hydroxy-2-(1H-indol-3-yl)ethyl)(methyl)amino)-methyl)-7-methyl-4-oxo-4,7-dihydrothieno[2,3-b]pyridine-5-carboxamide.

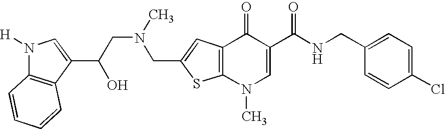

N-(4-Chlorobenzyl)-2-(chloromethyl)-7-methyl-4-oxo-4,7-dihydrothieno[2,3-b]pyridine-5-carboxamide (Preparation 1, 0.500 g) is suspended in DMF (30 mL), and N,N-diisopropylethylamine (0.34 mL) and 1-(3a,7a-dihydro-1H-indol-3-yl)-2-(methylamino)ethanol (Preparation 27, 0.375 g) are added. The reaction mixture is heated to 90° C. for 2 h. The reaction mixture is allowed to cool to room temperature and is poured into water (60 mL). The resulting brown solid is filtered and purified by column chromatography (CH₂Cl₂/methanol; 99/1, 98/2). The resulting yellow, gummy solid is recrystallized from ethyl acetate to yield 0.245 g of the title compound as a pale yellow solid. Physical characteristics. M.p. 120-123° C.; ¹H NMR (400 MHz, DMSO-d₆) δ 10.86, 10.62, 8.69, 7.53-7.51, 7.41-7.31, 7.22-7.21, 7.03-6.99, 6.88-6.85, 5.07-5.03, 4.85, 4.55, 3.90, 3.88, 2.85-2.81, 2.36; ¹³C NMR (100 MHz, CDCl₃) δ 173.0, 171.2, 165.0, 150.6, 144.6, 137.3, 136.5, 132.8, 131.5, 128.9, 128.7, 125.7, 122.3, 121.6, 119.7, 119.3, 116.5, 115.7, 111.3, 64.4, 63.4, 60.4, 57.3, 43.1, 42.6, 42.1, 21.1, 14.2; MS (ESI+) m/z 535 (M+H)⁺. Anal. Found: C, 61.85; H, 5.41; N, 9.61; Cl, 6.12; S, 5.53.

### Example 12

rac-N-(4-Chlorobenzyl)-2-(((2-(2,5-dimethyl-3-furyl)-2-hydroxyethyl)(methyl)-amino) methyl)-7-methyl-4-oxo-4,7-dihydrothieno[2,3-b]pyridine-5-carboxamide.

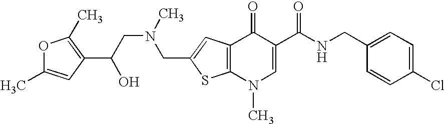

1-(2,5-Dimethyl-3-furyl)-2-(methylamino)ethanol (Preparation 29, 0.333 g) is dissolved in DMF (30 mL), and N,N-diisopropylethylamine (0.34 mL) and N-(4-chlorobenzyl)-2-(chloromethyl)-7-methyl-4-oxo-4,7-dihydrothieno[2,3-b]pyridine-5-carboxamide (Preparation 1, 0.500 g) are added. The reaction mixture is heated to 90° C. for 1 h. The reaction mixture is allowed to cool to room temperature and is poured into water (100 mL). The resulting off-white solid is filtered and recrystallized from acetonitrile to yield 0.550 g of a tan solid which is recrystallized from methanol to yield 0.337 g of the title compound as a pale yellow solid. Physical characteristics. M.p. 167-172° C.; ¹H NMR (400 MHz, DMSO-d₆) δ 10.61, 8.70, 7.41-7.31, 5.91, 4.75, 4.61-4.57, 4.55, 3.97, 3.83, 2.67-2.62, 2.54-2.46, 2.27, 2.17, 2.15; ¹³C NMR (100 MHz, CDCl₃) δ 173.0, 164.9, 150.6, 150.1, 146.9, 144.7, 137.3, 132.8, 131.5, 128.9, 128.7, 121.8, 119.6, 115.8, 104.4, 63.3, 62.5, 57.2, 43.2, 42.6, 41.8, 13.4, 11.8; MS (ESI+) m/z 515 (M+H)⁺. Anal. Found: C, 60.35; H, 5.47; N, 8.14.

### Example 13

rac-2-(((2-(1-Benzothien-3-yl)-2-hydroxyethyl)(methyl)amino)methyl)-N-(4-chlorobenzyl)-7-methyl-4-oxo-4,7-dihydrothieno[2,3-b]pyridine-5-carboxamide.

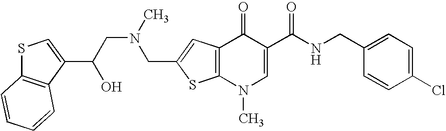

1-(3a,7a-Dihydro-1-benzothien-3-yl)-2-(methylamino)ethanol (Preparation 30, 0.408 g) is dissolved in DMF (30 mL), and N,N-diisopropylethylamine (0.34 mL) and N-(4-chlorobenzyl)-2-(chloromethyl)-7-methyl-4-oxo-4,7-dihydrothieno[2,3-b]pyridine-5-carboxamide (Preparation 1, 0.500 g) are added. The reaction mixture is heated to 90° C. for 1 h. The reaction mixture is allowed to cool to room temperature and is poured into water (60 mL). The resulting tan solid is filtered and recrystallized twice from DMF/H₂O to yield 0.468 g of the title compound as a white solid. Physical characteristics. M.p. 213-219° C. (dec); ¹H NMR (400 MHz, DMSO-d₆) δ 10.61, 8.67, 7.95-7.93, 7.81, 7.57, 7.41-7.33, 7.28-7.21, 5.39, 5.17-5.13, 4.55, 3.87, 3.84, 2.89-2.73, 2.39; MS (ESI+) m/z 552 (M+H)⁺. Anal. Found: C, 60.75; H, 4.75; N, 7.60; Cl, 6.46; S, 11.62.

### Example 14

rac-N-(4-Chlorobenzyl)-2-(((2-hydroxy-2-(1-methyl-1H-indol-2-yl)ethyl)(methyl)-amino)methyl)-7-methyl-4-oxo-4,7-dihydrothieno[2,3-b]pyridine-5-carboxamide.

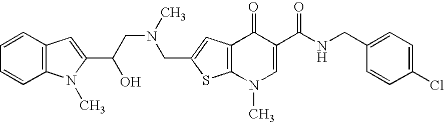

2-(Methylamino)-1-(1-methyl-1H-indol-2-yl)ethanol (Preparation 31, 0.100 g) is dissolved in DMF (10 mL), and N,N-diisopropylethylamine (0.085 mL) and N-(4-chlorobenzyl)-2-(chloromethyl)-7-methyl-4-oxo-4,7-dihydrothieno[2,3-b]pyridine-5-carboxamide (Preparation 1, 0.156 g) are added. The reaction mixture is heated to 90° C. for 2 h. The reaction mixture is allowed to cool to room temperature and is poured into water (25 mL). The resulting tan solid is filtered and purified by column chromatography (CH₂Cl₂/methanol, 99/1). The resulting yellow solid is recrystallized from ethyl acetate/methanol to yield 0.086 g of the title compound as a pale yellow solid. Physical characteristics. M.p. 128-132° C. (dec); ¹H NMR (400 MHz, DMSO-d₆) δ 10.60, 8.65, 7.47-7.45, 7.41-7.33, 7.12-7.08, 7.00-6.97, 6.35, 5.35, 5.03-4.99, 4.55, 3.91-3.83, 3.72, 3.71, 2.95-2.83, 2.37; ¹³C NMR (100 MHz, CDCl₃) δ 173.0, 164.9, 150.5, 144.6, 138.9, 137.9, 137.3, 132.8, 131.5, 128.9, 128.7, 127.2, 121.8, 120.7, 119.6, 115.8, 109.0, 99.3, 63.1, 61.3, 57.4, 43.0, 42.6, 42.3, 30.0; MS (ESI+) m/z 549 (M+H)⁺; HRMS (FAB) m/z 549.1710 (M+H)⁺. Anal. Found: C, 62.79; H, 5.49; N, 9.96; Cl, 6.38; S, 5.84.

### Example 15

rac-N-(4-Chlorobenzyl)-2-(((2-(5-cyanothien-2-yl)-2-hydroxyethyl)(methyl)-amino)methyl)-7-methyl-4-oxo-4,7-dihydrothieno[2,3-b]pyridine-5-carboxamide.

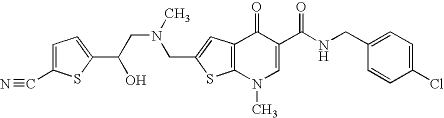

5-(1-Hydroxy-2-(methylamino)ethyl)thiophene-2-carbonitrile (Preparation 33, 0.182 g) is dissolved in DMF (15 mL), and N,N-diisopropylethylamine (0.17 mL) and N-(4-chlorobenzyl)-2-(chloromethyl)-7-methyl-4-oxo-4,7-dihydrothieno[2,3-b]pyridine-5-carboxamide (Preparation 1, 0.250 g) are added. The reaction mixture is heated to 90° C. for 2 h. The reaction mixture is allowed to cool to room temperature and is poured into water (30 mL) and extracted with CH₂Cl₂ (4×25 mL). The combined organic layers are dried (MgSO₄), filtered, and concentrated in vacuo. The resulting orange oil is purified by column chromatography (CH₂Cl₂/methanol, 99/1). The resulting pale yellow solid is recrystallized from ethyl acetate to yield 0.117 g of the title compound as a white solid. Physical characteristics. M.p. 155-160° C. (dec); ¹H NMR (400 MHz, DMSO-d₆) δ 10.60, 8.71, 7.85, 7.41-7.33, 7.15, 6.13, 5.07, 4.55, 3.94, 3.89, 2.72-2.70, 2.32; ¹³C NMR (100 MHz, CDCl₃)δ 173.0, 164.8, 154.1, 150.5, 144.8, 137.4, 137.3, 136.8, 132.8, 131.5, 128.9, 128.7, 123.3, 122.3, 115.9, 114.3, 108.6, 66.4, 64.4, 57.3, 43.2, 42.6, 41.9; MS (ESI+) m/z 527 (M+H)⁺. Anal. Found: C, 56.73; H, 4.44; N, 10.54; Cl, 6.75; S, 12.03.

### Example 16

rac-N-(4-Chlorobenzyl)-2-(((2-hydroxy-2-(1,3-thiazol-2-yl)ethyl)(methyl)amino)-methyl)-7-methyl-4-oxo-4,7-dihydrothieno[2,3-b]pyridine-5-carboxamide.

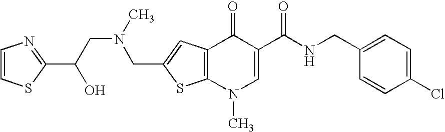

N-(4-Chlorobenzyl)-2-(chloromethyl)-7-methyl-4-oxo-4,7-dihydrothieno[2,3-b]-pyridine-5-carboxamide (Preparation 1, 0.08 g), DMF (2 mL) and N,N-diisopropyl-ethylamine (0.11 mL) are stirred with 4 Å molecular sieves (0.100 g) for 1 h and then 2-(methylamino)-1-(1,3-thiazol-2-yl)ethanol (Preparation 35, 0.050 g) is added. The mixture is heated at 85° C. for 3 h. Sat. aq. NH₄Cl is added. The mixture is extracted with CH₂Cl₂. The combined organic layers are dried (MgSO₄), filtered and concentrated. The crude product is recrystallized from acetonitrile to afford 0.059 g of the title compound as off-white crystals. Physical characteristics. M.p. 129-133° C.;

¹H NMR (400 MHz, DMSO-d₆) δ 10.61, 8.70, 7.73-7.72, 7.64-7.63, 7.41-7.39, 7.35-7.33, 7.33-7.31, 6.21-6.19, 5.12-5.02, 4.55-4.53, 3.93, 3.89, 2.94-2.90, 2.79-2.75, 2.34; MS (ESI+) m/z 503 (M+H)⁺; HRMS (FAB) m/z 503.0983 (M+H)⁺.

### Example 17

rac-2-(((2-(1,3-Benzothiazol-2-yl)-2-hydroxyethyl)(methyl)amino)methyl)-N-(4-chlorobenzyl)-7-methyl-4-oxo-4,7-dihydrothieno[2,3-b]pyridine-5-carboxamide.

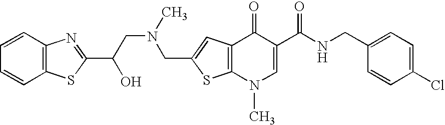

N-(4-Chlorobenzyl)-2-(chloromethyl)-7-methyl-4-oxo-4,7-dihydrothieno[2,3-b]-pyridine-5-carboxamide (Preparation 1, 0.20 g), DMF (5 mL) and N,N-diisopropyl-ethylamine (0.24 mL) are stirred with crushed 4 Å molecular sieves (0.200 g) for 1 h and then 1-(1,3-benzothiazol-2-yl)-2-(methylamino)ethanol (Preparation 37, 0.12 g) is added. The mixture is heated at 80° C. for 3 h. Water is added and the mixture is extracted with CH₂Cl₂. The combined organic layers are dried (MgSO₄), filtered and concentrated. The crude product is purified by chromatotron to afford 0.053 g of the title compound as an off-white solid. Physical characteristics. ¹H NMR (400 MHz, DMSO-d₆) δ 10.60, 8.66, 8.09-8.07, 7.93-7.91, 7.46-7.31, 6.45-6.44, 5.16, 4.55-4.53, 3.89, 3.73, 3.02-2.98, 2.92-2.87, 2.38; ¹³C NMR (CDCl₃) δ 174.5, 173.4, 165.3, 53.5, 150.9, 145.1, 137.7, 137.6, 135.0, 133.2, 131.9, 129.3, 129.0, 126.5, 125.4, 123.2, 122.5, 122.2, 116.2, 69.2, 62.8, 57.7, 43.5, 43.0, 42.5; MS (ESI+) m/z 553 (M+H)⁺; HRMS (FAB) m/z 553.1141 (M+H)⁺.

### Example 18

rac-N-(4-Chlorobenzyl)-2-(((2-hydroxy-2-(1H-pyrazol-5-yl)ethyl)(methyl)amino)-methyl)-7-methyl-4-oxo-4,7-dihydrothieno[2,3-b]pyridine-5-carboxamide.

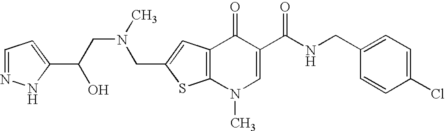

A mixture of 2-(methylamino)-1-(1H-pyrazol-5-yl)ethanol dihydrochloride (Preparation 39, 0.12 g), KI (0.02 g), triethylamine (0.16 mL), and 3 Å molecular sieves in dry DMF (15 mL) is stirred for 2 h. N-(4-Chlorobenzyl)-2-(chloromethyl)-7-methyl-4-oxo-4,7-dihydrothieno[2,3-b]pyridine-5-carboxamide (Preparation 1, 0.19 g) is added. The resulting mixture is stirred at room temperature overnight. The molecular sieves are removed by filtration and the filtrate is diluted with EtOAc (100 mL). The solution is wash with water (3×50 mL) and brine (50 mL), dried (Na₂SO₄), and concentrated. The crude product is purified by chromatatron (1000μ rotor, eluting with a CH₂Cl₂/CH₃OH/aq. NH₃; 90/9/1), followed by recrystallization from EtOAc to afford 0.07 g of the title compound as a white solid. Physical characteristics. M.p. 177.5-178.6° C.; ¹H NMR (400 MHz, DMSO-d₆) δ 12.53, 10.62, 8.70, 7.41-7.71, 6.16, 4.82, 4.54, 3.94, 3.89-3.73, 2.72, 2.30; ¹³C NMR (DMSO-d₆) δ 174.2, 166.7, 152.8, 147.4, 142.1, 140.9, 133.7, 132.9, 131.4, 130.7, 122.3, 116.6, 104.3, 65.3, 58.6, 45.1, 44.5, 43.7, 42.8, 42.8, 42.6; MS (ESI+) m/z 486 (M+H)⁺; Anal. Found: C, 56.60; H, 5.03; N, 14.33.

### Example 19

N-(4-Chlorobenzyl)-2-((((2R)-2-hydroxy-2-(1H-pyrazol-5-yl)ethyl)(methyl)amino)methyl)-7-methyl-4-oxo-4,7-dihydrothieno[2,3-b]pyridine-5-carboxamide.

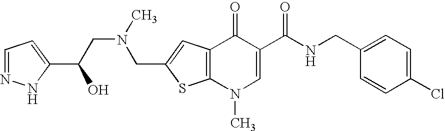

rac-N-(4-Chlorobenzyl)-2-(((2-hydroxy-2-(1H-pyrazol-5-yl)ethyl)(methyl)amino)-methyl)-7-methyl-4-oxo-4,7-dihydrothieno[2,3-b]pyridine-5-carboxamide (Example 18, 0.15 g) is separated by preparative HPLC (5×25 cm Chiralpak AD column, 90 mL/min. 0.2% DEA in ethanol, λ=230 nM) and fractions containing the slower eluting isomer are combined and concentrated. The crude product is recrystallized from methanol/EtOAc to afford 65 mg of the title compound as a white solid. Physical characteristics. M.p. 177.3-178.8° C.; ¹H NMR (400 MHz, DMSO-d₆) δ 12.53, 10.62, 8.70, 7.41-7.71, 6.16, 4.82, 4.54, 3.94, 3.89-3.73, 2.72, 2.30; ¹³C NMR (DMSO-d₆) δ 174.2, 166.7, 152.8, 147.4, 142.1, 140.9, 133.7, 132.9, 131.4, 130.7, 122.3, 116.6, 104.3, 65.3, 58.6, 45.1, 44.5, 43.7, 42.8, 42.8, 42.6.

### Example 20

N-(4-Chlorobenzyl)-7-ethyl-2-((((2R)-2-(2-furyl)-2-hydroxyethyl)(methyl)amino)-methyl)-4-oxo-4,7-dihydrothieno [2,3-b]pyridine-5-carboxamide.

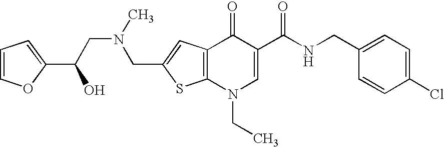

N,N-Diisopropylethylamine (0.22 mL) is add to a solution of N-(4-chlorobenzyl)-2-(chloromethyl)-7-ethyl-4-oxo-4,7-dihydrothieno[2,3-b]pyridine-5-carboxamide (Preparation 1, 250 mg) and (1R)-1-(2-furyl)-2-(methylamino)ethanol (Preparation 17, 178 mg) in DMF (14 mL). The mixture is stirred at 90° C. for 1 h and then allowed to cool to room temperature. The mixture is concentrated in vacuo and the resulting solid is recrystallized from methanol to afford 140 mg of the title compound as a white solid. Physical characteristics. M.p. 123-127° C.; ¹H NMR (300 MHz, DMSO-d₆) δ 10.62, 8.72, 7.56, 7.37, 7.30, 6.39, 6.28, 5.30, 4.75, 4.54, 4.28, 3.83, 2.76, 2.27, 1.43; HRMS (FAB) m/z 500.1396 (M+H)⁺.

### Example 21

N-(4-Chlorobenzyl)-2-((((2R)-2-(2-furyl)-2-hydroxyethyl)(methyl)amino)methyl)-7-(2-methoxyethyl)-4-oxo-4,7-dihydrothieno[2,3-b]pyridine-5-carboxamide.

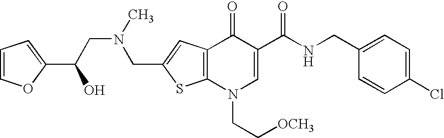

N,N-Diisopropylethylamine (0.203 mL) is added to a solution of N-(4-chlorobenzyl)-2-(chloromethyl)-7-(2-methoxyethyl)-4-oxo-4,7-dihydrothieno[2,3-b]pyridine-5-carboxamide (Preparation 1, 250 mg) and (1R)-1-(2-furyl)-2-(methylamino)ethanol (Preparation 17, 165 mg) in DMF (14 mL). The mixture is stirred at 90° C. for 1 h and then allowed to cool to room temperature. The mixture is concentrated in vacuo and the crude product is purified by chromatography, eluting with 1% MeOH/CHCl₃ to afford 142 mg of the title compound as a white solid. Physical characteristics. M.p. 121-122° C.; ¹H NMR (300 MHz, DMSO-d6) δ 10.58, 8.63, 7.56, 7.36, 7.29, 6.39, 6.29, 5.29, 4.75, 4.54, 4.43, 3.82, 3.73, 3.25, 2.76, 2.27.

### Example 22

rac-2-(((2-(1-Benzofuran-2-yl)-2-hydroxyethyl)(methyl)amino)methyl)-N-(4-chlorobenzyl)-7-ethyl-4-oxo-4,7-dihydrothieno[2,3-b]pyridine-5-carboxamide.

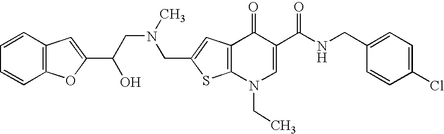

N,N-Diisopropylethylamine (0.22 mL) is added to a solution of N-(4-chlorobenzyl)-2-(chloromethyl)-7-ethyl-4-oxo-4,7-dihydrothieno[2,3-b]pyridine-5-carboxamide (Preparation 1, 250 mg) and 1-(1-benzofuran-2-yl)-2-(methylamino)ethanol (Preparation 21, 240 mg) in DMF (14 mL). The mixture is stirred at 90° C. for 1 h and then allowed to cool to room temperature. The mixture is concentrated in vacuo and the crude product is recrystallized from methanol to afford 272 mg of the title compound as a white solid. Physical characteristics. M.p. 142-145° C.; ¹H NMR (300 MHz, DMSO-d6) δ 10.60, 8.67, 7.61, 7.54, 7.36, 7.29, 7.23, 6.77, 5.60, 4.90, 4.54, 4.02, 3.87, 2.83, 2.32, 1.30. Anal. Found: C, 62.96; H, 5.08; N, 7.66; Cl, 6.53; S, 5.78.

### Example 23

rac-2-(((2-(1-Benzofuran-2-yl)-2-hydroxyethyl)(methyl)amino)methyl)-N-(4-chlorobenzyl)-7-propyl-4-oxo-4,7-dihydrothieno[2,3-b]pyridine-5-carboxamide.

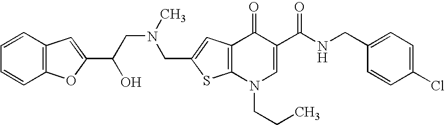

N,N-Diisopropylethylamine (0.213 mL) is added to a solution of N-(4-chlorobenzyl)-2-(chloromethyl)-7-propyl-4-oxo-4,7-dihydrothieno[2,3-b]pyridine-5-carboxamide (Preparation 1, 250 mg) and 1-(1-benzofuran-2-yl)-2-(methylamino)ethanol (Preparation 21, 230 mg) in DMF (14 mL). The mixture is stirred at 90° C. for 1 h and then allowed to cool to room temperature. The mixture is concentrated in vacuo and the crude product is recrystallized from methanol to afford 226 mg of the title compound as a white solid. Physical characteristics. M.p. 132-133° C.; ¹H NMR (300 MHz, DMSO-d6) δ 10.60, 8.66, 7.58, 7.45, 7.36, 7.29, 7.23, 6.77, 5.61, 4.95, 4.54, 4.05, 3.81, 2.93, 2.32, 1.70, 0.81. Anal. Found: C, 62.96; H, 5.08; N, 7.66; Cl, 6.53; S, 5.78.

### Example 24

rac-2-(((2-(1-Benzofuran-2-yl)-2-hydroxyethyl)(methyl)amino)methyl)-N-(4-chlorobenzyl)-7-(2-methoxyethyl)-4-oxo-4,7-dihydrothieno[2,3-b]pyridine-5-carboxamide.

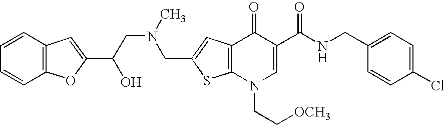

N,N-Diisopropylethylamine (0.203 mL) is added to a solution of N-(4-chlorobenzyl)-2-(chloromethyl)-7-(2-methoxyethyl)-4-oxo-4,7-dihydrothieno[2,3-b]pyridine-5-carboxamide (Preparation 1, 250 mg) and 1-(1-benzofuran-2-yl)-2-(methylamino)-ethanol (Preparation 21, 220 mg) in DMF (14 mL). The mixture is stirred at 90° C. for 2 h and then allowed to cool to room temperature. The mixture is concentrated in vacuo and triturated with ethyl acetate to afford 206 mg of the title compound as an off-white solid. Physical characteristics. M.p. 133-140° C.; ¹H NMR (300 MHz, DMSO-d6) δ 10.60, 8.59, 7.58, 7.46, 7.36, 7.29, 7.23, 6.77, 5.61, 4.95, 4.54, 4.25, 3.82, 3.61, 3.20, 2.89, 2.32; HRMS (FAB) m/z 580.1678 (M+H)⁺. Anal. Found: C, 61.74; H, 5.13; N, 7.13; Cl, 6.05; S, 5.42.

Preparation 41.

rac-2-(((2-(1-Benzofuran-2-yl)-2-hydroxyethyl)(methyl)amino)methyl)-N-(4-chlorobenzyl)-7-((2,2-dimethyl-1,3-dioxolan-4-yl)methyl)-4-oxo-4,7-dihydro-thieno[2,3-b]pyridine-5-carboxamide.

Cesium carbonate (260 mg) and 3 Å molecular sieves (100 mg) are added to a solution of 2-(chloromethyl)-N-((4-chlorophenyl)methyl)-7-((2,2-dimethyl-1,3-dioxolan-4-yl)-methyl)-4,7-dihydro-4-oxothieno[2,3-b]pyridine-5-carboxamide (Preparation 3, 336 mg) and 1-(1-benzofuran-2-yl)-2-(methylamino)ethanol (Preparation 21, 133 mg) in DMF (3.0 mL). The reaction mixture is placed on a shaker block at 60° C. for 17 h. The solvent is evaporated and the residue is purified by chromatography over silica gel with 5% MeOH in CH₂Cl₂. The crude product is recrystallized from EtOAc to afford 192 mg of the title compound as a white solid. Physical characteristics. ¹H NMR (400 MHz, DMSO-d₆) δ 10.58, 8.64, 7.59, 7.47, 7.39, 7.35, 7.29, 7.22, 6.77, 5.59, 4.90, 4.53, 4.41, 4.13, 4.05, 3.84, 3.68, 2.93, 2.79, 2.33, 1.29, 1.21; Anal. Found: C, 62.13; H, 5.60; N, 6.40.

### Example 25

rac-2-(((2-(1-Benzofuran-2-yl)-2-hydroxyethyl)(methyl)amino)methyl)-N-(4-chlorobenzyl)-7-(2,3-dihydroxypropyl)-4-oxo-4,7-dihydrothieno[2,3-b]pyridine-5-carboxamide.

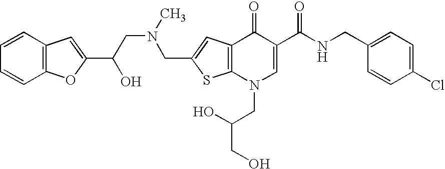

2-(((2-(1-Benzofuran-2-yl)-2-hydroxyethyl)(methyl)amino)methyl)-N-(4-chlorobenzyl)-7-((2,2-dimethyl-1,3-dioxolan-4-yl)methyl)-4-oxo-4,7-dihydrothieno[2,3-b]pyridine-5-carboxamide (Preparation 41, 127 mg) is dissolved in THF (3 mL) and 65% perchloric acid (0.2 mL) is added. The reaction mixture is stirred for 1 h at 50° C. and then poured into sat. NaHCO₃ solution. The mixture is extracted with EtOAc (100 mL) and the organic layer is dried (MgSO₄), filtered, and concentrated. The residue is chromatographed over silica gel with 5% MeOH in CH₂Cl₂. The crude product is triturated with EtOAc to afford 74 mg of the title compound as a white solid. Physical characteristics. ¹H NMR (400 MHz, DMSO-d₆) δ 10.55, 8.57, 7.58, 7.47, 7.39, 7.34, 7.31, 7.23, 6.76, 5.60, 5.28, 4.92, 4.53, 4.01, 3.83, 3.43, 3.32, 2.91, 2.79, 2.32; HRMS (FAB) m/z 596.1625 (M+H)⁺.

Preparation 42.

N-(4-Chlorobenzyl)-7-((2,2-dimethyl-1,3-dioxolan-4-yl)methyl)-2-((((2R)-2-(2-furyl)-2-hydroxyethyl)(methyl)amino)methyl)-4-oxo-4,7-dihydrothieno [2,3-b]-pyridine-5-carboxamide.

Cesium carbonate (230 mg) and 3 Å molecular sieves (100 mg) are added to a solution of 2-(chloromethyl)-N-((4-chlorophenyl)methyl)-7-((2,2-dimethyl-1,3-dioxolan-4-yl)-methyl)-4,7-dihydro-4-oxothieno[2,3-b]pyridine-5-carboxamide (Preparation 3, 200 mg) and (1R)-1-(2-furyl)-2-(methylamino)ethanol (Preparation 17, 100 mg) in DMF (3.0 mL). The reaction mixture is placed on a shaker block at 60° C. for 17 h. The solvent is evaporated and the residue is purified by chromatography over silica gel with 5% MeOH in CH₂Cl₂. to afford 145 mg of the title compound as a white solid.

Physical characteristics. ¹H NMR (400 MHz, DMSO-d₆) δ 10.58, 8.68, 7.55, 7.39, 7.34, 7.28, 6.38, 6.28, 5.26, 4.73, 4.53, 4.44, 4.27, 4.13, 3.81, 3.76, 2.77, 2.68, 1.33, 1.09; Anal. Found: C, 59.31; H, 5.59; N, 7.11.

### Example 26

N-(4-Chlorobenzyl)-7-(2,3-dihydroxypropyl)-2-((((2R)-2-(2-furyl)-2-hydroxy-ethyl)(methyl)amino)methyl)-4-oxo-4,7-dihydrothieno[2,3-b]pyridine-5-carboxamide.

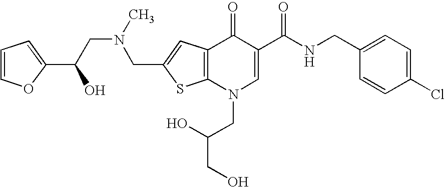

N-(4-Chlorobenzyl)-7-((2,2-dimethyl-1,3-dioxolan-4-yl)methyl)-2-((((2R)-2-(2-furyl)-2-hydroxyethyl)(methyl)amino)methyl)-4-oxo-4,7-dihydrothieno[2,3-b]pyridine-5-carboxamide (Preparation 421, 100 mg) is dissolved in THF (10 mL) and 65% perchloric acid (0.2 mL) is added. The reaction mixture is stirred for 6 h at room temperature and is then poured into sat. NaHCO₃ solution. The mixture is extracted with EtOAc (100 mL) and the organic laer is dried (MgSO₄), filtered and concentrated. The residue is chromatographed over silica gel with 5% MeOH in CH₂Cl₂ to afford 56 mg of the title compound as a white solid. Physical characteristics. ¹H NMR (400 MHz, DMSO-d₆) δ 10.51, 8.60, 7.55, 7.39, 7.33, 7.29, 6.38, 6.28, 5.31, 5.29, 4.98, 4.74, 4.53, 4.31, 4.12, 3.86, 3.80, 3.49, 3.38, 2.77, 2.66; HRMS (FAB) m/z 546.1463 (M+H)⁺.

Preparation 43.

N-(4-Chlorobenzyl)-2-((((2R)-2-(2-furyl)-2-hydroxyethyl)(methyl)amino)methyl)-4-oxo-7-(3-(tetrahydro-2H-pyran-2-yloxy)propyl)-4,7-dihydrothieno [2,3-b]-pyridine-5-carboxamide.

Cesium carbonate (225 mg) and 3 Å molecular sieves (100 mg) are added to a solution of N-(4-chlorobenzyl)-2-(chloromethyl)-4-oxo-7-(3-(tetrahydro-2H-pyran-2-yloxy)-propyl)-4,7-dihydrothieno[2,3-b]pyridine-5-carboxamide (Preparation 5, 200 mg) and (1R)-1-(2-furyl)-2-(methylamino)ethanol (Preparation 17, 100 mg) in DMF (2.0 mL). The reaction mixture is placed on a shaker block at 60° C. for 17 h. The solvent is evaporated and the residue is purified by chromatography over silica gel with 5% MeOH in CH₂Cl₂. to afford 177 mg of the title compound as a white solid. Physical characteristics. ¹H NMR (400 MHz, DMSO-d₆) δ 10.57, 8.68, 7.55, 7.39, 7.33, 7.29, 6.38, 6.28, 5.27, 4.73, 4.53, 4.48, 4.34, 3.82, 3.68, 3.38, 2.77, 2.27, 2.10, 1.62, 1.55, 1.17; HRMS (FAB) m/z 614.2105 (M+H)⁺.

### Example 27

N-(4-Chlorobenzyl)-2-((((2R)-2-(2-furyl)-2-hydroxyethyl)(methyl)amino)methyl)-7-(3-hydroxypropyl)-4-oxo-4,7-dihydrothieno[2,3-b]pyridine-5-carboxamide.

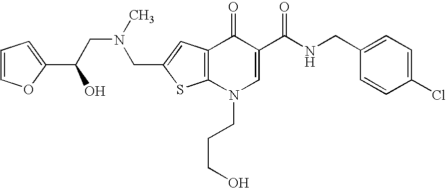

N-(4-Chlorobenzyl)-2-((((2R)-2-(2-furyl)-2-hydroxyethyl)(methyl)amino)methyl)-4-oxo-7-(3-(tetrahydro-2H-pyran-2-yloxy)propyl)-4,7-dihydrothieno[2,3-b]pyridine-5-carboxamide (Preparation 43, 112 mg) is dissolved in THF (10 mL) and 65% perchloric acid (0.2 mL) in water (0.2 mL) is added. The reaction mixture is stirred at 22° C. for 7 h and is then poured into sat. NaHCO₃ solution. The mixture is extracted with EtOAc (150 mL) and the organic layer is dried (MgSO₄), filtered and concentrated. The residue is chromatographed over silica gel with 5% MeOH in CH₂Cl₂ and the resulting crude product is crystallized from EtOAc/ether to afford 57 mg of the title compound. Physical characteristics. ¹H NMR (400 MHz, DMSO-d₆) δ 10.57, 8.68, 7.55, 7.39, 7.34, 7.29, 6.38, 6.28, 5.29, 4.76, 4.54, 4.30, 3.81, 3.45, 2.75, 2.27, 1.97; HRMS (FAB) m/z 530.1533 (M+H)⁺.

Preparation 44.

rac-2-(((2-(1-Benzofuran-2-yl)-2-hydroxyethyl)(methyl)amino)methyl)-N-(4-chlorobenzyl)-4-oxo-7-(3-(tetrahydro-2H-pyran-2-yloxy)propyl)-4,7-dihydro-thieno[2,3-b]pyridine-5-carboxamide.

Cesium carbonate (245 mg) and 3 Å molecular sieves (100 mg) are added to a solution of N-(4-chlorobenzyl)-2-(chloromethyl)-4-oxo-7-(3-(tetrahydro-2H-pyran-2-yloxy)-propyl)-4,7-dihydrothieno[2,3-b]pyridine-5-carboxamide (Preparation 5, 315 mg) and 1-(1-benzofuran-2-yl)-2-(methylamino)ethanol (Preparation 20, 143 mg) in DMF (2.0 mL). The reaction mixture is placed on a shaker block at 60° C. for 8 h. The solvent is evaporated and the residue is purified by chromatography over silica gel with 5% MeOH in CH₂Cl₂ to afford 156 mg of the title compound as a white solid. Physical characteristics. ¹H NMR (400 MHz, DMSO-d₆) δ 10.55, 8.65, 7.58, 7.45, 7.39, 7.33, 7.29, 7.22, 6.77, 5.60, 4.89, 4.53, 4.44, 4.18, 4.05, 3.83, 3.63, 3.33, 2.93, 2.80, 2.32, 1.97, 1.51, 1.35; HRMS m/z 664.2278 (M+H)⁺. Anal. Found: C, 63.06; H, 5.79; N, 6.26.

### Example 28

rac-2-(((2-(1-Benzofuran-2-yl)-2-hydroxyethyl)(methyl)amino)methyl)-N-(4-chlorobenzyl)-7-(3-hydroxypropyl)-4-oxo-4,7-dihydrothieno[2,3-b]pyridine-5-carboxamide.

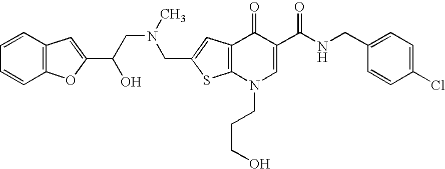

rac-2-(((2-(1-Benzofuran-2-yl)-2-hydroxyethyl)(methyl)amino)methyl)-N-(4-chloro-benzyl)-4-oxo-7-(3-(tetrahydro-2H-pyran-2-yloxy)propyl)-4,7-dihydrothieno[2,3-b]-pyridine-5-carboxamide (Preparation 44, 100 mg) is dissolved in THF (5 mL) and of 65% perchloric acid (0.2 mL) in water (0.2 mL) is added. The reaction mixture is stirred at 50° C. for 1 h and is then poured into sat. NaHCO₃ solution. The mixture is extracted with EtOAc (150 mL) and the organic layer is dried (MgSO₄), filtered and concentrated. The residue is chromatographed over silica gel with 5% MeOH in CH₂Cl₂ and the crude product is crystallized from EtOAc/ether to afford 87 mg of the title compound as white crystals. Physical characteristics. ¹H NMR (400 MHz, DMSO-d₆) δ 10.53, 8.63, 7.59, 7.46, 7.39, 7.34, 7.29, 7.22, 6.77, 5.60, 4.89, 4.72, 4.53, 4.14, 4.03, 3.83, 3.39, 2.92, 2.81, 2.32, 1.86; HRMS (FAB) m/z 580.1669 (M+H)⁺. Anal. Found: C, 61.74; H, 4.85; N, 7.02.

### Example 29

rac-2-(((2-(1-Benzofuran-2-yl)-2-hydroxyethyl)(methyl)amino)methyl)-N-(4-chlorobenzyl)-7-(2-hydroxyethyl)-4-oxo-4,7-dihydrothieno[2,3-b]pyridine-5-carboxamide.

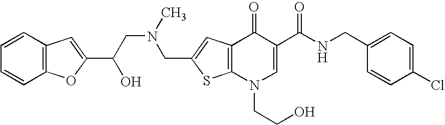

Cesium carbonate (260 mg) and 3 Å molecular sieves (100 mg) are added to a solution of N-(4-chlorobenzyl)-2-(chloromethyl)-7-(2-hydroxyethyl))-4-oxo-4,7-dihydro-thieno[2,3-b]pyridine-5-carboxamide (Preparation 8, 315 mg) and 1-(1-benzofuran-2-yl)-2-(methylamino)ethanol (Preparation 20, 133 mg) in DMF (3.0 mL). The reaction mixture is placed on a shaker block at 60° C. for 17 h. The solvent is evaporated and the residue is purified by chromatography over silica gel with 5% MeOH in CH₂Cl₂ to afford 158 mg of the title compound as a white solid. Physical characteristics. ¹H NMR (400 MHz, DMSO-d₆) δ 10.55, 8.58, 7.59, 7.47, 7.39, 7.33, 7.29, 6.77, 5.59, 5.09, 4.90, 4.54, 4.10, 4.00, 3.83, 3.66, 2.91, 2.81, 2.31, 1.09; HRMS m/z 566.1514 (M+H)⁺.

Preparation 45.

rac-5-(4,5-Dimethyl-2-furyl)-3-methyl-1,3-oxazolidin-2-one.

A solution of tert-butyl dimethylcarbamate (7.6 g) and tetramethylethylenediamine (13.4 mL) in THF (200 mL) is cooled to −70° C. and sec-butyl lithium (1.3 M in cyclohexane, 49.6 mL) is added maintaining temperature below −65° C. The mixture is allowed to stir at −70° C. for 1.25 h. A solution of 4,5-dimethylfurylcarboxaldehyde (5.0 g) in THF (20 mL) is added maintaining the temperature below −65° C. and stirring is continued for 2 h. The mixture is allowed to warm to to 0° C. and with ice-bath cooling is quenched with saturated aq. NH₄Cl (100 mL). The mixture is diluted with diethyl ether (300 mL). The aqueous layer is seperated and extracted with diethyl ether (2×100 mL). The combined organic layers are washed with saturated aq. NH₄Cl (2×50 mL) followed by brine (50 mL), dried (MgSO₄), and concentrated. The residue is dissolved in THF (100 mL) and sodium hydride (60% dispersion in mineral oil, 3.23 g) is added. The mixture is allowed to stir at room temperature for 18 h and with ice-bath cooling is quenched with saturated aq. NH₄Cl (100 mL). The mixture is extracted with diethyl ether (200 mL). The organic layer is washed with saturated aq. NH₄Cl (100 mL) followed by brine (100 mL), dried (MgSO₄) and concentrated. The crude product is purified by column chromatography (heptane/EtOAc, 4/1; 1/1) to afford 3.46 g of the title compound as a brown oil. Physical characteristics. ¹H NMR (400 MHz, DMSO-d₆) δ 6.44, 5.46, 3.78, 3.64, 2.80, 2.19, 1.90; ¹³C NMR (100 MHz, DMSO-d₆) δ 157.4, 148.8, 147.6, 115.1, 113.8, 67.4, 49.6, 30.9, 11.5, 9.8. Anal. Found: C, 61.29; H, 6.87; N, 7.35.

Preparation 46.

rac-1-(4,5-Dimethyl-2-furyl)-2-(methylamino)ethanol.

A mixture of rac-5-(4,5-dimethyl-2-furyl)-3-methyl-1,3-oxazolidin-2-one (Preparation 45, 3.23 g), ethanol (10 mL), and a solution of 1 M aq. potassium hydroxide (58 mL) is heated to 60° C. for 7 h. The mixture is allowed to cool to room temperature, is sturated with NaCl, and extracted with diethyl ether (4×100 mL). The combined organic layers are concentrated to 100 mL and extracted with saturated aq. NH₄Cl (6×50 mL). The combined aqueous layers are adjusted to pH 10 with solid sodium hydroxide and are extracted with diethyl ether (5×100 mL). The combined organic layers are dried (K₂CO₃/Na₂SO₄) and concentrated. The residue is dissolved in diethyl ether, filtered through Celite, and concentrated. The crude product is crystallized from hexanes/EtOAc (10/1) at −10° C. to afford 1.04 g of the title compound as a yellow solid. Physical characteristics. M.p. 59.5-60° C.; ¹H NMR (400 MHz, DMSO-d₆) δ 6.00, 5.16, 4.47, 2.69, 2.64, 2.28, 2.14, 1.86; ¹³C NMR (100 MHz, CDCl₃) δ 151.8, 143.2, 111.7, 106.7, 62.9, 54.1, 33.8, 9.0, 7.5; MS (ESI+) m/z 170 (M+H)⁺. Anal. Found: C, 63.63; H, 8.78; N, 8.01.

### Example 30

rac-N-(4-Chlorobenzyl)-2-(((2-(4,5-dimethyl-2-furyl)-2-hydroxyethyl)(methyl)-amino)methyl)-7-methyl-4-oxo-4,7-dihydrothieno[2,3-b]pyridine-5-carboxamide.

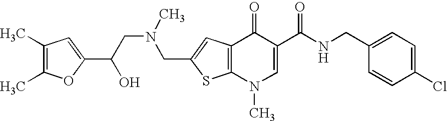

rac-1-(4,5-Dimethyl-2-furyl)-2-(methylamino)ethanol (Preparation 46, 0.169 g) is dissolved in DMF (15 mL), and N,N-diisopropylethylamine (0.17 mL) and N-(4-chlorobenzyl)-2-(chloromethyl)-7-methyl-4-oxo-4,7-dihydrothieno[2,3-b]pyridine-5-carboxamide (Preparation 1, 0.191 g) are added. The reaction mixture is heated to 90° C. for 2 h. The reaction mixture is allowed to cool to room temperature and is poured into water (50 mL) and extracted with EtOAc (3×100 mL). The combined organic layers are dried (Na₂SO₄) and concentrated in vacuo. The crude product is purified by column chromatography (CH₂Cl₂/methanol, 50/1; 33/1) to afford 0.201 g of the title compound as a white solid. Physical characteristics. M.p. 166-167° C.; 11H NMR (400 MHz, DMSO-d₆) δ 10.62, 8.70, 7.41-7.33, 7.31, 6.03, 5.13, 4.61, 4.55, 3.94, 3.84, 3.78, 2.74, 2.68, 2.27, 2.11, 1.87; ¹³C NMR (100 MHz, CDCl₃) δ 172.3, 164.8, 154.0, 150.8, 145.7, 145.5, 140.1, 139.0, 131.7, 130.9, 129.5, 128.7, 120.4, 114.6, 114.2, 109.6, 64.8, 61.5, 56.6, 43.1, 42.6, 41.7, 11.5, 10.0; MS (ESI+) m/z 514 (M+H)⁺. Anal. Found: C, 60.76; H, 5.58; N, 8.22; Cl, 6.85; S, 6.23.

Preparation 47.

rac-3-Methyl-5-(5-phenyl-2-furyl)-1,3-oxazolidin-2-one.

A solution of tert-butyl dimethylcarbamate (5.47 g) and tetramethylethylenediamine (9.6 mL) in THF (160 mL) is cooled to −70° C. and sec-butyl lithium (1.3 M in cyclohexane, 35.7 mL) is added maintaining temperature below −65° C. The mixture is allowed to stir at −70° C. for 1.25 h. A solution of 5-phenylfurylcarboxaldehyde (5.0 g) in THF (20 mL) is added maintaining the temperature below −65° C. and stirring is continued for 2 h. The mixture is allowed to warm to to 0° C. and with ice-bath cooling is quenched with saturated aq. NH₄Cl (100 mL). The mixture is diluted with diethyl ether (300 mL). The aqueous layer is seperated and extracted with diethyl ether (2×100 mL). The combined organic layers are washed with saturated aq. NH₄Cl (2×50 mL) followed by brine (50 mL), dried (MgSO₄), and concentrated. The residue is dissolved in THF (100 mL) and sodium hydride (60% dispersion in mineral oil, 2.32 g) is added. The mixture is allowed to stir at room temperature for 18 h and with ice-bath cooling is quenched with saturated aq. NH₄Cl (100 mL). The mixture is extracted with diethyl ether (200 mL). The organic layer is washed with saturated aq. NH₄Cl (100 mL) followed by brine (100 mL), dried (MgSO₄) and concentrated. The crude product is purified by column chromatography (heptane/EtOAc, 4/1; 1/1) to afford 3.48 g of the title compound as a tan solid. Physical characteristics. M.p. 97-99° C.; ¹H NMR (400 M z, DMSO-d₆) δ 7.73, 7.45, 7.33, 7.00, 6.81, 5.65, 3.88, 3.82, 2.86; ¹³C NMR (100 MHz, DMSO-d₆) δ 158.1, 155.6, 149.4, 130.5, 129.1, 128.4, 124.4, 112.6, 106.2, 68.2, 50.9, 31.5; MS (CI) m/z 244 (M+H)⁺. Anal. Found: C, 69.04; H, 5.49; N, 5.74.

Preparation 48.

rac-2-(Methylamino)-1-(5-phenyl-2-furyl)ethanol.

A mixture of rac-3-methyl-5-(5-phenyl-2-furyl)-1,3-oxazolidin-2-one (Preparation 47, 2.43 g), ethanol (20 mL), and a solution of 1 M aq. potassium hydroxide (35 mL) is heated to 50° C. for 7 h. The mixture is allowed to cool to room temperature, is sturated with NaCl, and extracted with diethyl ether (4×100 mL). The combined organic layers are concentrated to 100 mL and extracted with saturated aq. NH₄Cl (6×50 mL). The combined aqueous layers are adjusted to pH 10 with solid sodium hydroxide and are extracted with diethyl ether (5×100 mL). The combined organic layers are dried (K₂CO₃/Na₂SO₄) and concentrated. The crude product is crystallized from diethyl ether to afford 1.56 g of the title compound as a white solid. Physical characteristics. M.p. 75-76° C.; ¹H NMR (400 MHz, DMSO-d₆) δ 7.67, 7.41, 7.27, 6.86, 6.38, 5.42, 4.66, 2.84-2.76, 2.32; ¹³C NMR (100 MHz, CDCl₃) δ 155.4, 153.8, 131.1, 129.0, 127.7, 124.1, 108.9, 106.0, 65.8, 55.9, 36.3; MS (ESI+) m/z 218 (M+H)⁺. Anal. Found: C, 71.54; H, 6.96; N, 6.40.

### Example 31

rac-N-(4-Chlorobenzyl)-2-(((2-(5-phenyl-2-furyl)-2-hydroxyethyl)(methyl)-amino)methyl)-7-methyl-4-oxo-4,7-dihydrothieno[2,3-b]pyridine-5-carboxamide.

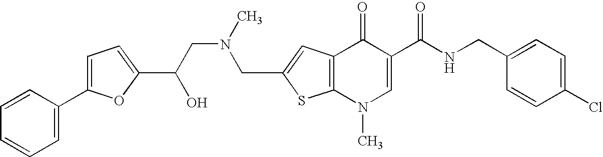

rac-2-(Methylamino)-1-(5-phenyl-2-furyl)ethanol (Preparation 48, 0.217 g) is dissolved in DMF (15 mL), and N,N-diisopropylethylamine (0.17 mL) and N-(4-chlorobenzyl)-2-(chloromethyl)-7-methyl-4-oxo-4,7-dihydrothieno[2,3-b]pyridine-5-carboxamide (Preparation 1, 0.191 g) are added. The reaction mixture is heated to 90° C. for 2 h. The reaction mixture is allowed to cool to room temperature and is poured into water (50 mL) and extracted with EtOAc (3×100 mL). The combined organic layers are dried (Na₂SO₄) and concentrated in vacuo. The crude product is purified by column chromatography (CH₂Cl₂/methanol, 100/1; 50/1) and crystallized from EtOAc/CH₂Cl₂/diethyl ether to afford 0.217 g of the title compound as a white solid. Physical characteristics. M.p. 177-178° C.; ¹H NMR (400 MHz, DMSO-d₆) δ 10.61, 8.51, 7.53, 7.42-7.28, 7.17, 6.88, 6.41, 5.40, 4.75, 4.55, 3.85, 3.77, 3.68, 2.94, 2.70, 2.34; ¹³C NMR (100 MHz, CDCl₃) δ 170.9, 163.4, 155.6, 150.6, 149.4, 143.9, 138.9, 137.7, 130.4, 129.4, 128.1, 127.7, 127.4, 126.0, 121.9, 119.0, 113.3, 107.8, 105.3, 63.7, 60.0, 55.5, 41.8, 41.4, 40.4; MS (ESI+) m/z 562 (M+H)⁺. Anal. Found: C, 63.93; H, 5.13; N, 7.47; Cl, 6.29; S, 5.66.

### Example 32

N-(4-Chlorobenzyl)-2-((((2R)-2-(2-furyl)-2-hydroxyethyl)(methyl)amino)methyl)-4-oxo-7-propyl-4,7-dihydrothieno[2,3-b]pyridine-5-carboxamide.

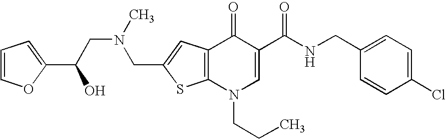

A mixture of N-(4-chlorobenzyl)-2-(chloromethyl)-7-ethyl-4-oxo-4,7-dihydrothieno[2,3-b]pyridine-5-carboxamide (Preparation 12, 0.25 g), (1R)-1-(2-furyl)-2-(methylamino)ethanol (Preparation 17, 0.17 g) and N,N-diisopropylethylamine (0.21 mL) in DMF (14 mL) is heated at 90° C. for 1 h. The reaction mixture is allowed to cool to room temperature and is then concentrated in vacuo. The crude product is purified by column chromatography (CH₂Cl₂/methanol, 97/3) and then triturated with diethyl ether to afford 0.24 g of the title compound as an off-white powder. Physical characteristics. ¹H NMR (300 MHz, DMSO-d₆) δ 10.60, 8.69, 7.56, 7.43-7.26, 6.39, 6.29, 5.70, 4.73, 4.55, 4.22, 3.81, 2.74, 2.29, 1.85, 0.89; HRMS (ESI) m/z 514.1588 (M+H)⁺.

Preparation 49.

5-(5-Chloro-2-furyl)-3-methyl-1,3-oxazolidin-2-one.

A mixture of tert-butyl dimethylcarbamate (7.2 g) and N,N,N′,N′-tetramethylethylene-diamine (12.7 mL) in THF (210 mL) is cooled to −70° C. sec-Butyl lithium (1.4 M in cyclohexane, 43.7 mL) is added dropwise, maintaining the reaction temperature below −65° C. The mixture is allowed to stir for 1.5 h at −70° C. A solution of 5-chloro-2-furaldehyde (5.0 g) in THF (20 mL) is added dropwise, maintaining the reaction temperature below −65° C. The mixture is allowed to stir for 1 h at −70° C. The mixture is allowed to warm to 0 DC and is then quenched with sat. aq. NH₄Cl solution (125 mL) while cooled by an ice bath. The mixture is diluted with diethyl ether (300 mL). The aqueous layer is extracted with diethyl ether (2×100 mL). The combined organic layers are washed with sat. aq. NH₄Cl (2×50 mL) followed by brine (50 mL), dried (MgSO₄), and concentrated. The resulting oil is dissolved in THF (115 mL) and sodium hydride (60% dispersion in mineral oil, 3.1 g) is added. The mixture is allowed to stir at room temperature for 18 h. With an ice bath cooling the reaction mixture is quenched with sat. aq. NH₄Cl solution (100 mL). The mixture is diluted with diethyl ether (200 mL). The organic layer is washed with sat. aq. NH₄Cl (100 mL) followed by brine (100 mL), dried (MgSO₄), and concentrated in vacuo. The crude product is purified by column chromatography (EtOAc/heptane, 1/1) to afford 1.9 g of the title compound as an amber oil. Physical characteristics. ¹H NMR (400 MHz, DMSO-d₆) δ 6.80, 6.55, 5.56, 3.81, 3.69, 2.81.

Preparation 50.

1-(5-Chloro-2-furyl)-2-(methylamino)ethanol.

5-(5-chloro-2-furyl)-3-methyl-1,3-oxazolidin-2-one (Preparation 49, 2.0 g) is dissolved in ethanol (20 mL) and a solution of 1 M aq. KOH (35 mL) is added. The mixture is heated to reflux for 4 h. The mixture is allowed to cool to room temperature, NaCl is added, and the reaction mixture is extracted with diethyl ether. The organic layers are concentrated in vacuo to 100 mL volume and are extracted with saturated. aq. NH₄Cl (6×50 mL). The combined aqueous layers are adjusted to pH 10 with solid NaOH, then extracted with diethyl ether (5×100 mL). The combined organic layers are dried (Na₂SO₄) and concentrated to afford the title compound as a brown crystalline solid. Physical characteristics. ¹H NMR (300 MHz, DMSO-d₆) δ 6.35, 4.54, 2.69, 2.28.

### Example 33

N-(4-Chlorobenzyl)-2-(((2-(5-chloro-2-furyl)-2-hydroxyethyl)(methyl)amino)-methyl)-7-methyl-4-oxo-4,7-dihydrothieno[2,3-b]pyridine-5-carboxamide.

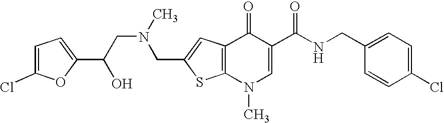

A mixture of N-(4-chlorobenzyl)-2-(chloromethyl)-7-methyl-4-oxo-4,7-dihydro-thieno[2,3-b]pyridine-5-carboxamide (Preparation 1, 0.16 g), 1-(5-chloro-2-furyl)-2-(methylamino)ethanol (Preparation 50, 0.15 g) and N,N-diisopropylethylamine (0.15 mL) in DMF (12 mL) is heated to 90° C. for 2 h. The reaction mixture is cooled to room temperature and diluted with water (20 mL). The suspension is filtered and the resulting solid is recrystallized from methanol to afford 0.11 g of the title compound as an off-white powder. Physical characteristics. ¹H NMR (300 MHz, DMSO-d₆) δ 10.62, 8.70, 7.45-7.27, 6.41, 5.44, 4.68, 4.53, 4.11, 3.92, 3.81, 3.18, 2.85-2.60, 2.27. Anal. Found: C, 55.19; H, 4.53; N, 8.01; Cl, 13.33; S, 6.10.

Preparation 51.

N-(4-Chlorobenzyl)-2-((((2R)-2-(2-furyl)-2-hydroxyethyl)(methyl)amino)methyl)-4-oxo-7-(2-(tetrahydro-2H-pyran-2-yloxy)ethyl)-4,7-dihydrothieno[2,3-b]-pyridine-5-carboxamide.

Cesium carbonate (260 mg) and 3 Å molecular sieves (100 mg) are added to a solution of N-(4-chlorobenzyl)-2-(chloromethyl)-4-oxo-7-(2-(tetrahydro-2H-pyran-2-yloxy)ethyl)-4,7-dihydrothieno[2,3-b]pyridine-5-carboxamide (Preparation 7, 315 mg) and (1R)-1-(2-furyl)-2-(methylamino)ethanol (Preparation 17, 133 mg) in DMF (3.0 mL). The reaction mixture is placed on a shaker block at 60° C. for 17 h. The solvent is evaporated and the residue is purified by chromatography over silica gel with 5% MeOH in CH₂Cl₂ to afford the title compound. Physical characteristics. ¹H NMR (400 MHz, DMSO-d₆) δ 10.56, 8.71, 7.56, 7.40-7.29, 6.39, 6.28, 5.28, 4.74, 4.58, 4.45, 3.96, 3.78, 3.34, 3.30, 2.76, 2.50, 2.26, 1.53, 1.38, 1.27. Anal. Found: C, 59.76; H, 5.80; N, 6.95.

### Example 34

N-(4-Chlorobenzyl)-2-((((2R)-2-(2-furyl)-2-hydroxyethyl)(methyl)amino)methyl)-7-(2-hydroxyethyl)-4-oxo-4,7-dihydrothieno[2,3-b]pyridine-5-carboxamide.

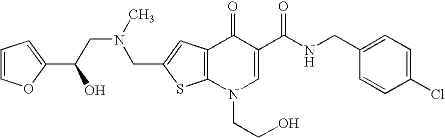

A 62% solution of perchloric acid (100 μL) is added to a solution of N-(4-chlorobenzyl)-2-((((2R)-2-(2-furyl)-2-hydroxyethyl)(methyl)amino)methyl)-4-oxo-7-(2-(tetrahydro-2H-pyran-2-yloxy)ethyl)-4,7-dihydrothieno[2,3-b]pyridine-5-carboxamide (Preparation 51, 120 mg) in THF (5 mL). The reaction mixture is stirred at 22° C. for 1 h. The mixture is diluted with EtOAc (150 mL) and washed with saturated aq. sodium bicarbonate solution. The organic layer is dried (MgSO₄), filtered, and concentrated to afford 79 mg of the title compound. Physical characteristics. ¹H NMR (400 MHz, DMSO-d₆) δ 10.6, 8.62, 7.56, 7.40, 7.38, 7.29, 6.39, 6.28, 5.28, 5.15, 4.74, 4.55, 4.28, 3.86-3.76, 3.33, 2.76, 2.27; MS (CI) m/z 516 (M+H)⁺; HRMS (ESI) m/z 516.1368 (M+H)⁺.

Preparation 52.

N-(4-Chlorobenzyl)-2-(hydroxymethyl)-4-oxo-7-(2-(2-(tetrahydro-2H-pyran-2-yloxy)ethoxy)ethyl)-4,7-dihydrothieno[2,3-b]pyridine-5-carboxamide.

Cesium carbonate (3.25 g) is added to a solution of N-(4-chlorobenzyl)-4-hydroxy-2-(hydroxymethyl)thieno[2,3-b]pyridine-5-carboxamide (3.5 g, prepared as described in U.S. Pat. No. 6,239,142) and 2-(2-(2-chloroethoxy)ethoxy)tetrahydro-2H-pyran (2.1 g) in DMF (12 mL). The mixture is heated at 100° C. for 6 hours. The solvent is evaporated and the residue is purified by column chromatographed (CH₂Cl₂/methanol, 95/1) to afford 2.65 g of the title compound as a pale yellow solid. Physical characteristics. ¹H NMR (400 MHz, DMSO-d₆) δ 10.54, 8.66, 7.40-7.28, 5.80, 4.69, 4.53, 4.46, 4.41, 3.85, 3.60, 3.54, 3.48, 3.37, 3.23, 1.51-1.31; MS (CI) m/z 521 (M+H)⁺. Anal Found: C, 57.58; H, 5.61; N, 5.36.

Preparation 53.

N-(4-Chlorobenzyl)-2-(chloromethyl)-4-oxo-7-(2-(2-(tetrahydro-2H-pyran-2-yloxy)ethoxy)ethyl)-4,7-dihydrothieno[2,3-b]pyridine-5-carboxamide.

2,4,6-Collidine (1.6 mL) and 4-N,N-dimethylaminopyridine (20 mg) is added to a suspension of N-(4-chlorobenzyl)-2-(hydroxymethyl)-4-oxo-7-(2-(2-(tetrahydro-2H-pyran-2-yloxy)ethoxy)ethyl)-4,7-dihydrothieno[2,3-b]pyridine-5-carboxamide (Preparation 52, 2.62 g) in THF (15 mL). Methanesulfonyl chloride (0.78 mL) is added and the reaction is heated at 50° C. for 1 h. The solvent is evaporated and the residue dissolved in chloroform. The organic layer is washed with water, dried (MgSO₄), and concentrated. The crude product is purified by column chromatography (CH₂Cl₂/methanol, 95/5) and then crystallized from EtOAc to afford 1.8 g of the title compound as a tan solid. Physical characteristics. ¹H NMR (400 MHz, DMSO-d₆) δ 10.54, 8.70, 7.55, 7.40-7.33, 5.14, 4.53, 4.47, 4.39, 3.86, 3.61-3.46, 3.37, 3.33, 3.22, 1.49-1.31. Anal. Found: C, 55.65; H, 5.19; N, 5.11.

Preparation 54.

N-(4-Chlorobenzyl)-2-((((2R)-2-(2-furyl)-2-hydroxyethyl)(methyl)amino)methyl)-4-oxo-7-(2-(2-(tetrahydro-2H-pyran-2-yloxy)ethoxy)ethyl)-4,7-dihydrothieno[2,3-b]pyridine-5-carboxamide.

Diisopropylethylamine (192 EL), 3 Å molecular sieves (100 mg) are added to a solution of N-(4-chlorobenzyl)-2-(chloromethyl)-4-oxo-7-(2-(2-(tetrahydro-2H-pyran-2-yloxy)ethoxy)ethyl)-4,7-dihydrothieno[2,3-b]pyridine-5-carboxamide (Preparation 53, 270 mg) and (1R)-1-(2-furyl)-2-(methylamino)ethanol (Preparation 17, 141 mg) in DMF (2.5 mL). The reaction mixture is placed on a shaker block at room temperature for 17 h. The solvent is evaporated and the residue is purified by column chromatography over silica gel (CH₂Cl₂/methanol, 95/5) to afford the title compound. Physical characteristics. ¹H NMR (400 MHz, DMSO-d₆) δ 10.54, 8.64, 7.95, 7.56, 7.39, 7.33, 7.29, 6.39, 6.28, 5.28, 4.73, 4.53, 4.41, 3.83, 3.61, 3.54, 3.51, 3.39, 3.25, 2.89, 2.77, 2.73, 2.27, 1.30; MS (CI) m/z 644 (M+H)⁺; HRMS (ESI) m/z 644.2198 (M+H)⁺.

### Example 35

N-(4-Chlorobenzyl)-2-((((2R)-2-(2-furyl)-2-hydroxyethyl)(methyl)amino)methyl)-7-(2-(2-hydroxyethoxy)ethyl)-4-oxo-4,7-dihydrothieno[2,3-b]pyridine-5-carboxamide.

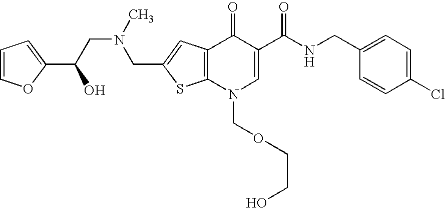

A 62% solution of perchloric acid (100 μL) is added to a solution of N-(4-chlorobenzyl)-2-((((2R)-2-(2-furyl)-2-hydroxyethyl)(methyl)amino)methyl)-4-oxo-7-(2-(2-(tetrahydro-2H-pyran-2-yloxy)ethoxy)ethyl)-4,7-dihydrothieno[2,3-b]pyridine-5-carboxamide (Preparation 54, 200 mg) in THF (5 mL). The reaction mixture is stirred at 22° C. for 2 h. The mixture is diluted with EtOAc (150 mL) and washed with saturated aq. sodium bicarbonate solution. The organic layer is dried (MgSO₄), filtered, and concentrated. The crude product is purified by column chromatography (chloroform/methanol, 95/5) to afford 118 mg of the title compound. Physical characteristics. ¹H NMR (400 MHz, DMSO-d₆) 6); 10.6, 8.66, 7.56, 7.39, 7.34, 7.29, 6.38, 6.29, 5.29, 4.73, 4.59, 4.53, 4.41, 3.84, 3.80, 3.43, 3.33, 2.75, 2.27; MS (CI) m/z 560 (M+H)⁺. Anal Found: C, 57.52; H, 5.65; N, 7.19.

Preparation 55.

2-(Methyl(trityl)amino)-1-(1-trityl-1H-imidazol-2-yl)ethanol.

A solution of 1-trityl imidazole (1.67 g) in 60 mL of THF is cooled to −78° C. and treated with a solution of n-BuLi (2.5 M in hexanes, 2.15 mL) dropwise under a nitrogen atmosphere. The resulting mixture is stirred for 30 min and then a solution of 2-N-tritylmethylamino acetadehyde (1.0 g) THF (10 mL) is added. The reaction mixture is stirred for 1 h at −79° C. and then allowed to warm to room temperature. The mixture is quenched by adding saturated aq. NH₄Cl and water dropwise. The resulting suspension is extracted into EtOAc (2×100 mL). The combined organic phase is washed with water (100 mL) followed by brine (50 mL), dried (Na₂SO₄), filtered, and concentrated in vacuo. The residue is purified by column chromatograph (CH₂Cl₂/EtOAc, 9/1) to afford 0.93 g of the title compound as a white solid. Physical characteristics. ¹H NMR (400 MHz, CDCl₃) δ 7.38-6.99, 6.61, 4.28-4.24, 3.51, 3.30-3.24, 1.33.

Preparation 56.

1-(1H-Imidazol-2-yl)-2-(methylamino)ethanol Dihydrochloride.

A solution of 2-(methyl(trityl)amino)-1-(1-trityl-1H-imidazol-2-yl)ethanol (Preparation 55, 3.4 g) in acetone (70 mL) is treated with a solution of 4 N HCl in dioxane (5 mL) and stirred for 4 h at room temperature. The solvent is removed by evaporation at reduced pressure and the residue is suspended in EtOAc. The resulting solids are collected by filtration and washed with hot EtOAc to afford 0.92 g of the title compound as a white solid. Physical characteristics. M.p. 176.4-177.3° C.; ¹H NMR (400 MHz, DMSO-d₆) δ 7.65, 7.40, 5.42, 3.64-3.39, 2.61; ¹³C NMR (DMSO-d₆) δ 146.2, 119.9, 61.4, 51.3, 33.2.

### Example 36

rac-N-(4-Chlorobenzyl)-2-(((2-hydroxy-2-(1H-imidazol-2-yl)ethyl)(methyl)-amino)methyl)-7-methyl-4-oxo-4,7-dihydrothieno[2,3-b]pyridine-5-carboxamide.

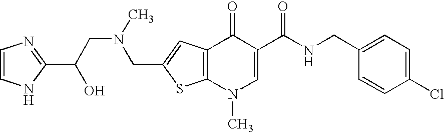

A mixture of rac-2-(2-methylamino-1-hydroxyethyl)imidazole dihydrochloride (Preparation 56, 0.17 g), KI (0.02 g), triethylamine (0.27 mL), and 3 Å molecular sieves in dry DMF (15 mL) is stirred for 2 h and treated with N-(4-chlorobenzyl)-2-(chloromethyl)-7-methyl-4-oxo-4,7-dihydrothieno[2,3-b]pyridine-5-carboxamide (Preparation 1, 0.3 g). The resulting mixture is stirred at ambient temperature overnight. The molecular sieves are removed by filtration and the filtrate is diluted with EtOAc (100 mL). The mixture is wash with water (3×50 mL) followed by brine (50 mL), dried (Na₂SO₄), and concentrated in vacuo. The crude product is purified by radial chromatography on 1000μ rotor, eluting with a CH₂Cl₂/methanol/aq. NH₃ (90/9/1), followed by recrystallization from methanol/EtOAc to afford 0.12 g of the title compound as a white solid. Physical characteristics. M.p. 177.9-179.5° C.; ¹H NMR (400 MHz, DMSO-d₆) δ 11.93, 10.62, 8.70, 7.41-7.30, 6.90, 5.44, 4.79, 4.54, 3.94, 3.89-3.78, 2.87-2.72, 2.50, 2.29; ¹³C NMR (DMSO-d₆) δ 170.5, 163.0, 149.1, 148.1, 143.7, 138.3, 137.2, 129.9, 129.2, 127.7, 126.9, 118.6, 112.9, 64.4, 60.4, 54.9, 41.4, 40.8, 39.9; MS (ESI+) m/z 486 (M+H)⁺.

1. A compound of formula I 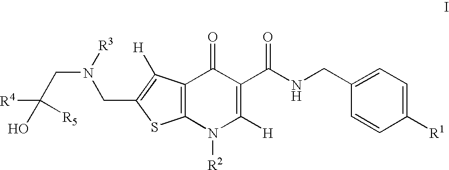
its enantiomeric, diasteromeric or tautomeric isomer, or a pharmaceutically acceptable salt thereof wherein,
R¹ is
(a) Cl,
(b) Br,
(c) F, or
(d) CN;
R² is
(a) C₁₋₄alkyl optionally substituted by one or more OH or C₁₋₄alkoxy, or
(b) (CH₂)ₘOCH₂CH₂OH;
R³ is C₁₋₂alkyl;
R⁴ is a five-(5) membered heteroaryl bonded via a carbon atom having one (1), two (2), or three (3) heteroatoms selected from the group consisting of O, S(O)ₘ, and N—W, wherein R⁴ is optionally fused to a benzene or pyridine ring, and optionally substituted with one or more R⁶;
wherein W is absence, H, or C₁₋₄alkyl;
R⁵ is
(a) H, or
(b) C₁₋₂alkyl optionally substituted by OH;
R⁶ is
(a) halo,
(b) OCF₃,
(c) cyano,
(d) nitro,
(e) CONR¹R⁸,
(f) NR⁷R⁸,
(g) C₁₋₇alkyl, which is optionally partially unsaturated and is optionally substituted by one or more R⁹,
(h) O(CH₂CH₂O)ₙR¹⁰,
(i) OR¹⁰,
(j) CO₂R¹⁰, or
(k) phenyl optionally substituted by halo, C₁₋₇alkyl, or C₁₋₇alkoxy;
R⁷ and R⁸ are independently
(a) H,
(b) phenyl optionally substituted by halo, C₁₋₇alkyl, or C₁₋₇alkoxy,
(c) C₁₋₇alkyl which is optionally substituted by one or more OR¹⁰, phenyl, or halo substituents,
(d) C₃₋₈cycloalkyl,
(e) (C═O)R¹¹, or
(f) R⁷ and R⁸ together with the nitrogen to which they are attached form a het, wherein het is a five-(5), or six-(6) membered heterocyclic ring having 1, 2, or 3 heteroatoms selected from the group consisting of oxygen, sulfur, or nitrogen, wherein het is optionally substituted with C₁₋₄ alkyl;
R⁹ is
(a) oxo,
(b) phenyl optionally substituted by halo, C₁₋₇alkyl, or C₁₋₇alkoxy,
(c) OR¹⁰,
(d) O(CH₂CH₂)OR¹⁰,
(e) SR¹⁰,
(f) NR⁷R⁸,
(g) halo,
(h) CO₂R¹⁰,
(i) CONR¹⁰OR¹⁰, or
(j) C₃₋₈cycloalkyl optionally substituted by OR¹⁰;
R¹⁰ is
(a) H,
(b) C₁₋₇alkyl,
(c) C₃₋₈cycloalkyl, or
(d) phenyl optionally substituted by halo, C₁₋₇alkyl, or C₁₋₇alkoxy;
R¹¹ is
(a) C₁₋₇alkyl,
(b) C₃₋₈cycloalkyl, or
(c) phenyl optionally substituted by halo, C₁₋₇alkyl, or C₁₋₇alkoxy;
n is 1, 2, 3, 4 or 5; and
each m is independently 1 or 2.

2. A compound of claim 1 which is a compound of formula IA

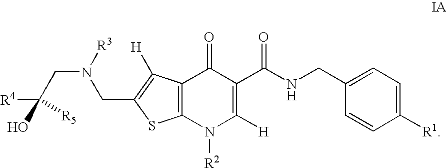

3. A compound of claim 1 or 2 wherein R¹ is chloro.

4. A compound of claim 1 or 2 wherein R² is C₁₋₃alkyl.

5. A compound of claim 1 or 2 wherein R² is methyl.

6. A compound of claim 1 or 2 wherein R² is C₁₋₃alkyl substituted with one or two hydroxy.

7. A compound of claim 1 or 2 wherein R² is C₁₋₄alkyl substituted by C₁₋₄alkoxy.

8. A compound of claim 1 or 2 wherein R² is CH₂CH₂OCH₂CH₂OH.

9. A compound of claim 1 or 2 wherein R³ is methyl.

10. A compound of claim 1 or 2 wherein R³ is ethyl.

11. A compound of claim 1 or 2 wherein R⁴ is a five-(5) membered heteroaryl bonded via a carbon atom having one (1) or two (2) heteroatoms selected from the group consisting of O, S, and N—W.

12. A compound of claim 11 wherein R⁴ is substituted by R⁶.

13. A compound of claim 11 wherein R⁴ is 2-furyl, 3-furyl, thien-2-yl, 1H-pyrrol-2-yl, 1-methyl-1H-pyrrol-2-yl, 1-methyl-1H-imidazol-4-yl, 1H-imidazol-4-yl, 1,3-thiazol-2-yl, or 1H-pyrazol-5-yl.

14. A compound of claim 13 wherein R⁴ is 2-furyl.

15. A compound of claim 12 wherein R⁴ is 5-methyl-2-furyl, 2,5-dimethyl-3-furyl, 5-phenyl-2-furyl, 5-chloro-2-furyl, 4,5-dimethyl-2-furyl, or 5-cyanothien-2-yl.

16. A compound of claim 1 or 2 wherein R⁴ is a five-(5) membered heteroaryl bonded via a carbon atom having one (1) or two (2) heteroatoms selected from the group consisting of O, S, and N—W, wherein R⁴ is fused to a benzene or pyridine ring.

17. A compound of claim 16 wherein R⁴ is substituted by R⁶.

18. A compound of claim 16 wherein R⁴ is benzofuran-2-yl, benzothien-3-yl, 1H-indol-3-yl, 1-methyl-1H-indol-2-yl, or 1,3-benzothiazol-2-yl.

19. A compound of claim 18 wherein R⁴ is benzofuran-2-yl.

20. A compound of claim 1 or 2 wherein R is hydrogen.

21. A compound of claim 12 wherein R⁶ is OH, halo, C₁₋₄alkyl, C₁₋₄alkoxy, cyano, nitro, OCF₃, NR⁷R⁸, phenyl, or CONR⁷R⁸.

22. A compound of claim 17 wherein R⁶ is OH, halo, C₁₋₄alkyl, C₁₋₄alkoxy, cyano, nitro, OCF₃, NR⁷R⁸, phenyl, or CONR⁷R⁸.

23. A compound of claim 21 wherein R⁶ is methyl.

24. A compound of claim 22 wherein R⁶ is methyl.

25. A compound of claim 21 wherein R⁷ and R⁸ together with the nitrogen to which they are attached form a het, wherein het is morpholine, piperidine, piperazine, or pyrrolidine.

26. A compound of claim 22 wherein R⁷ and R⁸ together with the nitrogen to which they are attached form a het, wherein het is morpholine, piperidine, piperazine, or pyrrolidine.

27. A pharmaceutical composition comprising a compound of claim 1 and a pharmaceutically acceptable carrier.

28. A method of treating infections by herpesviruses which comprises administering to a mammal in need thereof a compound of claim 1 or 2.

29. The method of claim 28 wherein said herpesviruses is herpes simplex virus types 1, herpes simplex virus types 2, varicella zoster virus, human cytomegalovirus, Epstein-Barr virus, human herpes virus 6, human herpes virus 7 or human herpes virus 8.

30. The method of claim 28 wherein said herpesviruses is human cytomegalovirus.

31. The method of claim 28 wherein said herpesviruses is varicella zoster virus or Epstein-Barr virus.

32. The method of claim 28 wherein said herpesviruses is herpes simplex virus types 1 or herpes simplex virus types 2.

33. The method of claim 28 wherein the compound of claim 1 is administered orally, parenterally or topically.

34. The method of claim 28 wherein the compound of claim 1 is in an amount of from about 0.1 to about 300 mg/kg of body weight.

35. The method of claim 28 wherein the compound of claim 1 is in an amount of from about 1 to about 30 mg/kg of body weight.

36. The method of claim 28 wherein said mammal is a human.

37. The method of claim 28 wherein said mammal is an animal.

38. A method of treating atherosclerosis and restenosis comprising administering to a mammal in need thereof a compound of claim 1 or 2.

39. A method for inhibiting a herpesviral DNA polymerase, comprising contacting the polymerase with an effective inhibitory amount of a compound of claim 1.

40. A compound of formula I, or a pharmaceutically acceptable salt thereof, for use in the manufacture of medicines for the treatment or prevention of a herpesviral infection in a mammal.

41. A compound of claim 1 which is
(1) rac-N-(4-chlorobenzyl)-2-(((2-(2-furyl)-2-hydroxyethyl)(methyl)amino) methyl)-7-methyl-4-oxo-4,7-dihydrothieno[2,3-b]pyridine-5-carboxamide,
(2) (+)-N-(4-chlorobenzyl)-2-((((R)-2-(2-furyl)-2-hydroxyethyl)(methyl)amino) methyl)-7-methyl-4-oxo-4,7-dihydrothieno[2,3-b]pyridine-5-carboxamide,
(3) rac-N-(4-chlorobenzyl)-2-(((2-hydroxy-2-(5-methyl-2-furyl)ethyl)(methyl)-amino)methyl)-7-methyl-4-oxo-4,7-dihydrothieno[2,3-b]pyridine-5-carboxamide,
(4) rac-N-(4-chlorobenzyl)-2-(((2-(3-furyl)-2-hydroxyethyl)(methyl)amino)-methyl)-7-methyl-4-oxo-4,7-dihydrothieno[2,3-b]pyridine-5-carboxamide,
(5) rac-2-(((2-(1-benzofuran-2-yl)-2-hydroxyethyl)(methyl)amino)methyl)-N-(4-chlorobenzyl)-7-methyl-4-oxo-4,7-dihydrothieno[2,3-b]pyridine-5-carboxamide,
(6) rac-N-(4-chlorobenzyl)-2-(((2-hydroxy-2-thien-2-ylethyl)(methyl)amino)-methyl)-7-methyl-4-oxo-4,7-dihydrothieno [2,3-b]pyridine-5-carboxamide,
(7) rac-N-(4-chlorobenzyl)-2-(((2-hydroxy-2-(1H-pyrrol-2-yl)ethyl)(methyl)-amino)methyl)-7-methyl-4-oxo-4,7-dihydrothieno[2,3-b]pyridine-5-carboxamide,
(8) rac-N-(4-chlorobenzyl)-2-(((2-hydroxy-2-(1-methyl-1H-pyrrol-2-yl)ethyl)-(methyl)amino)methyl)-7-methyl-4-oxo-4,7-dihydrothieno[2,3-b]pyridine-5-carboxamide,
(9) rac-N-(4-chlorobenzyl)-2-(((2-hydroxy-2-(1-methyl-1H-imidazol-4-yl)ethyl)-(methyl)amino)methyl)-7-methyl-4-oxo-4,7-dihydrothieno[2,3-b]pyridine-5-carboxamide,
(10) rac-N-(4-chlorobenzyl)-2-(((2-hydroxy-2-(1H-imidazol-4-yl)ethyl) (methyl) amino)methyl)-7-methyl-4-oxo-4,7-dihydrothieno[2,3-b]pyridine-5-carboxamide,
(11) rac-N-(4-chlorobenzyl)-2-(((2-hydroxy-2-(1H-indol-3-yl)ethyl)(methyl)-amino)methyl)-7-methyl-4-oxo-4,7-dihydrothieno[2,3-b]pyridine-5-carboxamide,
(12) rac-N-(4-chlorobenzyl)-2-(((2-(2,5-dimethyl-3-furyl)-2-hydroxyethyl)-(methyl)amino) methyl)-7-methyl-4-oxo-4,7-dihydrothieno[2,3-b]pyridine-5-carboxamide,
(13) rac-2-(((2-(1-benzothien-3-yl)-2-hydroxyethyl)(methyl)amino)methyl)-N-(4-chlorobenzyl)-7-methyl-4-oxo-4,7-dihydrothieno[2,3-b]pyridine-5-carboxamide,
(14) rac-N-(4-chlorobenzyl)-2-(((2-hydroxy-2-(1-methyl-1H-indol-2-yl)ethyl)-(methyl)amino)methyl)-7-methyl-4-oxo-4,7-dihydrothieno[2,3-b]pyridine-5-carboxamide,
(15) rac-N-(4-chlorobenzyl)-2-(((2-(5-cyanothien-2-yl)-2-hydroxyethyl)(methyl)-amino)methyl)-7-methyl-4-oxo-4,7-dihydrothieno[2,3-b]pyridine-5-carboxamide,
(16) rac-N-(4-chlorobenzyl)-2-(((2-hydroxy-2-(1,3-thiazol-2-yl)ethyl)(methyl)-amino)methyl)-7-methyl-4-oxo-4,7-dihydrothieno[2,3-b]pyridine-5-carboxamide,
(17) rac-2-(((2-(1,3-benzothiazol-2-yl)-2-hydroxyethyl)(methyl)amino)methyl)-N-(4-chlorobenzyl)-7-methyl-4-oxo-4,7-dihydrothieno[2,3-b]pyridine-5-carboxamide,
(18) rac-N-(4-chlorobenzyl)-2-(((2-hydroxy-2-(1H-pyrazol-5-yl)ethyl)(methyl)-amino)methyl)-7-methyl-4-oxo-4,7-dihydrothieno[2,3-b]pyridine-5-carboxamide,
(19) N-(4-chlorobenzyl)-2-((((2R)-2-hydroxy-2-(1H-pyrazol-5-yl)ethyl)(methyl)-amino)methyl)-7-methyl-4-oxo-4,7-dihydrothieno[2,3-b]pyridine-5-carboxamide,
(20) N-(4-chlorobenzyl)-7-ethyl-2-((((2R)-2-(2-furyl)-2-hydroxyethyl)(methyl)-amino)methyl)-4-oxo-4,7-dihydrothieno[2,3-b]pyridine-5-carboxamide,
(21) N-(4-chlorobenzyl)-2-((((2R)-2-(2-furyl)-2-hydroxyethyl)(methyl)amino)-methyl)-7-(2-methoxyethyl)-4-oxo-4,7-dihydrothieno[2,3-b]pyridine-5-carboxamide,
(22) rac-2-(((2-(1-benzofuran-2-yl)-2-hydroxyethyl)(methyl)amino)methyl)-N-(4-chlorobenzyl)-7-ethyl-4-oxo-4,7-dihydrothieno[2,3-b]pyridine-5-carboxamide,
(23) rac-2-(((2-(1-benzofuran-2-yl)-2-hydroxyethyl)(methyl)amino)methyl)-N-(4-chlorobenzyl)-7-propyl-4-oxo-4,7-dihydrothieno[2,3-b]pyridine-5-carboxamide,
(24) rac-2-(((2-(1-benzofuran-2-yl)-2-hydroxyethyl)(methyl)amino)methyl)-N-(4-chlorobenzyl)-7-(2-methoxyethyl)-4-oxo-4,7-dihydrothieno[2,3-b]pyridine-5-carboxamide,
(25) rac-2-(((2-(1-benzofuran-2-yl)-2-hydroxyethyl)(methyl)amino)methyl)-N-(4-chlorobenzyl)-7-(2,3-dihydroxypropyl)-4-oxo-4,7-dihydrothieno[2,3-b]pyridine-5-carboxamide,
(26) N-(4-chlorobenzyl)-7-(2,3-dihydroxypropyl)-2-((((2R)-2-(2-furyl)-2-hydroxyethyl)(methyl)amino)methyl)-4-oxo-4,7-dihydrothieno[2,3-b]pyridine-5-carboxamide,
(27) N-(4-chlorobenzyl)-2-((((2R)-2-(2-furyl)-2-hydroxyethyl)(methyl)amino)-methyl)-7-(3-hydroxypropyl)-4-oxo-4,7-dihydrothieno[2,3-b]pyridine-5-carboxamide,
(28) rac-2-(((2-(1-benzofuran-2-yl)-2-hydroxyethyl)(methyl)amino)methyl)-N-(4-chlorobenzyl)-7-(3-hydroxypropyl)-4-oxo-4,7-dihydrothieno[2,3-b]pyridine-5-carboxamide,
(29) rac-2-(((2-(1-benzofuran-2-yl)-2-hydroxyethyl)(methyl)amino)methyl)-N-(4-chlorobenzyl)-7-(2-hydroxyethyl)-4-oxo-4,7-dihydrothieno[2,3-b]pyridine-5-carboxamide,
(30) rac-N-(4-chlorobenzyl)-2-(((2-(4,5-dimethyl-2-furyl)-2-hydroxyethyl)(methyl)-amino)methyl)-7-methyl-4-oxo-4,7-dihydrothieno[2,3-b]pyridine-5-carboxamide,
(31) rac-N-(4-chlorobenzyl)-2-(((2-(5-phenyl-2-furyl)-2-hydroxyethyl)(methyl)-amino)methyl)-7-methyl-4-oxo-4,7-dihydrothieno[2,3-b]pyridine-5-carboxamide,
(32) N-(4-chlorobenzyl)-2-((((2R)-2-(2-furyl)-2-hydroxyethyl)(methyl)amino)-methyl)-4-oxo-7-propyl-4,7-dihydrothieno[2,3-b]pyridine-5-carboxamide,
(33) N-(4-chlorobenzyl)-2-(((2-(5-chloro-2-furyl)-2-hydroxyethyl)(methyl)amino)-methyl)-7-methyl-4-oxo-4,7-dihydrothieno[2,3-b]pyridine-5-carboxamide,
(34) N-(4-chlorobenzyl)-2-((((2R)-2-(2-furyl)-2-hydroxyethyl)(methyl)amino)-methyl)-7-(2-hydroxyethyl)-4-oxo-4,7-dihydrothieno[2,3-b]pyridine-5-carboxamide,
(35) N-(4-chlorobenzyl)-2-((((2R)-2-(2-furyl)-2-hydroxyethyl)(methyl)amino)-methyl)-7-(2-(2-hydroxyethoxy)ethyl)-4-oxo-4,7-dihydrothieno[2,3-b]pyridine-5-carboxamide,
(36) rac-N-(4-chlorobenzyl)-2-(((2-hydroxy-2-(1H-imidazol-2-yl)ethyl)(methyl)-amino)methyl)-7-methyl-4-oxo-4,7-dihydrothieno[2,3-b]pyridine-5-carboxamide, or a pharmaceutically acceptable salt thereof.

42. A compound of claim 1 which is (+)-N-(4-chlorobenzyl)-2-((((R)-2-(2-furyl)-2-hydroxyethyl)(methyl)amino)methyl)-7-methyl-4-oxo-4,7-dihydrothieno[2,3-b]-pyridine-5-carboxamide, or a pharmaceutically acceptable salt thereof.
